SEQUENCE LISTING (64 sequences)

SEQ ID NO: 1 - protein, 107 aa, Homo sapiens
MSESSSKSSQPLASKQEKDGTEKRGRGRPRKQPPVSPGTALVGSQKEPSEVPTPKRPRGRPKGSKNKGAAKTRKTTTTPGRKPRGRPKKLEKEEEEGISQESSEEEQ

SEQ ID NO: 2 - protein, 96 aa, Homo sapiens
MSESSSKSSQPLASKQEKDGTEKRGRGRPRKQPPKEPSEVPTPKRPRGRPKGSKNKGAAKTRKTTTTPGRKPRGRPKKLEKEEEEGISQESSEEEQ

SEQ ID NO: 3 - protein, 109 aa, Homo sapiens
MSARGEGAGQPSTSAQGQPAAPAPQKRGRGRPRKQQQEPTGEPSPKRPRGRPKGSKNKSPSKAAQKKAEATGEKRPRGRPRKWPQQVVQKKPAQEETEETSSQESAEED

SEQ ID NO: 4 - protein, 83 aa, Homo sapiens
MSARGEGAGQPSTSAQGQPAAPAPQKRGRGRPRKQQQEPTGEPSPKRPRGRPKGSKNKSPSKAAQKKAEATGEKRPRGRPRKW

SEQ ID NO: 5 - protein, 90 aa, Homo sapiens
MSARGEGAGQPSTSAQGQPAAPAPQKRGRGRPRKQQQEPTGEPSPKRPRGRPKGSKNKSPSKAAQKKAEATGEKRPRGRPRKWEEFYIAA

SEQ ID NO: 6 - protein, 96 aa, Homo sapiens
MSARGEGAGQPSTSAQGQPAAPAPQKRGRGRPRKQQQEPTGEPSPKRPRGRPKGSKNKSPSKAAQKKAEATGEKRPRGRPRKWPTIALCTHWINIC

SEQ ID NO: 7 - protein, 215 aa, Homo sapiens
MGKGDPKKPRGKMSSYAFFVQTCREEHKKKHPDASVNFSEFSKKCSERWKTMSAKEKGKFEDMAKADKARYEREMKTYIPPKGETKKKFKDPNAPKRPPSAFFLFCSEYRPKIKGEHPGLSIGDVAKKLGEMWNNTAADDKQPYEKKAAKLKEKYEKDIAAYRAKGKPDAAKKGVVKAEKSKKKKEEEEDEEDEEDEEEEEDEEDEDEEEDDDDE

SEQ ID NO: 8 - protein, 147 aa, Homo sapiens
MSARGEGAGQPSTSAQGQPAAPAPQKRGRGRPRKQQQEPTGEPSPKRPRGRPKGSKNKSPSKAAQKKAEATGEKRPRGRPRKWAGVQWYNLGSLQPPPPRFKQFSCLRLLSSWDYRHPPPHPANFCIFSRDRVSPCWPGWSRTPDLR

SEQ ID NO: 9 - protein, 106 aa, Homo sapiens
MSARGEGAGQPSTSAQGQPAAPAPQKRGRGRPRKQQQEPTGEPSPKRPRGRPKGSKNKSPSKAAQKKAEATGEKRPRGRPRKWDNLLPRTSSKKKTSLGNSTKRSH

SEQ ID NO: 10 - protein, 92 aa, Homo sapiens
MSARGEGAGQPSTSAQGQPAAPAPQKRGRGRPRKQQQEPTGEPSPKRPRGRPKGSKNKSPSKAAQKKAEATGEKRPRGRPRKWWLLMKSPCW

SEQ ID NO: 11 - protein, 96 aa, Homo sapiens
MSARGEGAGQPSTSAQGQPAAPAPQKRGRGRPRKQQQEPTGEPSPKRPRGRPKGSKNKSPSKAAQKKAEATGEKRPRGRPRKWPQQVVQKKPAQYS

SEQ ID NO: 12 - protein, 118 aa, Homo sapiens
MSARGEGAGQPSTSAQGQPAAPAPQKRGRGRPRKQQQEPTGEPSPKRPRGRPKGSKNKSPSKAAQKKAEATGEKRPRGRPRKWPQQVVQKKPAQVNVALPGKDHPGNLIYLLFSKNAT

SEQ ID NO: 13 - protein, 95 aa, Homo sapiens
MSARGEGAGQPSTSAQGQPAAPAPQKRGRGRPRKQQQEPTGEPSPKRPRGRPKGSKNKSPSKAAQKKAEATGEKRPRGRPRKWPQQVVQKKPAQD

SEQ ID NO: 14 - protein, 11 aa, Homo sapiens
TEKRGRGRPRK

SEQ ID NO: 15 - protein, 11 aa, Homo sapiens
TPKRPRGRPKG

SEQ ID NO: 16 - protein, 12 aa, Homo sapiens
TPGRKPRGRPKK

SEQ ID NO: 17 - protein, 11 aa, Homo sapiens
TEKRGRGRPRK

SEQ ID NO: 18 - protein, 11 aa, Homo sapiens
TPKRPRGRPKG

SEQ ID NO: 19 - protein, 12 aa, Homo sapiens
TPGRKPRGRPKK

SEQ ID NO: 20 - protein, 11 aa, Homo sapiens
PQKRGRGRPRK

SEQ ID NO: 21 - protein, 11 aa, Homo sapiens
SPKRPRGRPKG

SEQ ID NO: 22 - protein, 21 aa, Homo sapiens
TGEKRPRGRPRKWPQQVVQKK

SEQ ID NO: 23 - protein, 78 aa, Homo sapiens
PKKPRGKMSSYAFFVQTCREEHKKKHPDASVNFSEFSKKCSERWKTMSAKEKGKFEDMAKADKARYEREMKTYIPPKG

SEQ ID NO: 24 - protein, 71 aa, Homo sapiens
PRGKMSSYAFFVQTCREEHKKKHPDASVNFSEFSKKCSERWKTMSAKEKGKFEDMAKADKARYEREMKTYI

SEQ ID NO: 25 - protein, 73 aa, Homo sapiens
PKKPRGKMSSYAFFVQTCREEHKKKHPDASVNFSEFSKKCSERWKTMSAKEKGKFEDMAKADKARYEREMKTY

SEQ ID NO: 26 - protein, 75 aa, Homo sapiens
PNAPKRPPSAFFLFCSEYRPKIKGEHPGLSIGDVAKKLGEMWNNTAADDKQPYEKKAAKLKEKYEKDIAAYRAKG

SEQ ID NO: 27 - protein, 69 aa, Homo sapiens
PKRPPSAFFLFCSEYRPKIKGEHPGLSIGDVAKKLGEMWNNTAADDKQPYEKKAAKLKEKYEKDIAAYR

SEQ ID NO: 28 - protein, 49 aa, Homo sapiens
PKRPPSAFFLFCSEYRPKIKGEHPGLSIGDVAKKLGEMWNNTAADDKQP

SEQ ID NO: 29 - protein, 181 aa, Homo sapiens
EEHKKKNPDASVKFSEFLKKCSETWKTIFAKEKGKFEDMAKADKAHYEREMKTYIPPKGEKKKKFKDPNAPKRPPLAFFLFCSEYRPKIKGEHPGLSIDDVVKKLAGMWNNTAAADKQFYEKKAAKLKEKYKKDIAAYRAKGKPNSAKKRVVKAEKSKKKKEEEEDEEDEQEEENEEDDDK

SEQ ID NO: 30 - protein, 225 aa, Homo sapiens
MSARGEGAGQPSTSAQGQPAAPAPQKRGRGRPRKQQQEPTGEPSPKRPRGRPKGSKNKSPSKAAQKKAEATGEKRPRGRPRKWNTLEQCNVCSKPIMERILRATGKAYHPHCFTCVMCHRSLDGIPFTVDAGGLIHCIEDFHKKFAPRCSVCKEPIMPAPGQEETVRIVALDRDFHVHCYRCEDCGGLLSEGDNQGCYPLDGHILCKTCNSARIRVLTAKASTDL

SEQ ID NO: 31 - DNA, 1873 bp, Homo sapiens
  misc_feature: NCIB Accession No. M23614
gagcacgcggcggcggcggtctctgagcgcctctgctctctctcccggtttcagatccgcatttgctaccagcggcggccgcgcggagccaggccggtcctcagcgcccagcacggctcccggcaacccggagcgcgcaccgcagccggcggccgagctcgcgcatcccagccatcactcttccacctgctccttagagaagggaagatgagtgagtcgagctcgaagtccagccagcccttggcctccaagcaggaaaaggacggcactgagaagcggggccggggcaggccgcgcaagcagcctccggtgagtcccgggacagcgctggtagggagtcagaaggagcccagcgaagtgccaacacctaagagacctcggggccgaccaaagggaagcaaaaacaagggtgctgccaagacccggaaaaccaccacaactccaggaaggaaaccaaggggcagacccaaaaaactggagaaggaggaagaggagggcatctcgcaggagtcctcggaggaggagcagtgacccatgcgtgccgcctgctcctcactggaggagcagcttccttctgggactggacagctttgctccgctcccaccgcccccgccccttccccaggcccaccatcaccaccgcctctggccgccacccccatcttccacctgtgccctcaccaccacactacacagcacaccagccgctgcaggggctcccatgggcctgagtggggagcagttttcccctggcctcagttcacagctccccccgcccacccacgcatacacacatgccctcctggacaaggctaacatcccacttagccgcaccctgcacctgctgcgtccccactcccttggtggtggggacattgctctctgggcttttggtttgggggcgccctctctgctccttcactgttccctctggcttcccatagtggggcctgggagggttcccctggccttaaaaggggcccaagccatctcatcctggcacgccctactccactgccctggcacagcaggtgtggccaatggaggggggtgctggcccccaggattcccccagccaaactgtctttgtcaccacgtggggctcacttttcatccttccccaacttccctagtccccgtactaggttggacagcccccttcggctacaggaaggcaggaggggtgagtcccctactccctcttcactgtggccacagcccccttgccctccgcctgggatctgagtacatattgtggtgatggagatgcagtcacttattgtccaggtgaggcccaagagccctgtggccgcacctgaggtgggctggggctgctcccctaaccctactttcgttccgccactcagccatttccccctcctcagatggggcaccaataacaaggagctcaccctgcccgctcccaacccccctcctgctcctccctgccccccaaggttctgggttccatttttcctctgttcacaaactacctctggacagttgtgttgttttttgttcaatgttccattcttcgacatccgtcattgctgctgctaccagcgccaaatgttcatcctcattgcctcctgttctgcccacgatcccctcccccaagatactctttgtgggaagaggggctggggcatggcaggctgggtgaccgactaccccagtcccagggaaggtggccctgcccctaggatgctgcagcagagtgagcaagggggcccgaatcgaccataaagggtgtaggggccacctcctccccctgttctgttggggaggggtagccatgatttgtcccagcctggggctccctctctggtttcctatttgcagttacttgaataaaaaaaatatccttttctgg

SEQ ID NO: 32 - DNA, 324 bp, Homo sapiens
atgagtgagtcgagctcgaagtccagccagcccttggcctccaagcaggaaaaggacggcactgagaagcggggccggggcaggccgcgcaagcagcctccggtgagtcccgggacagcgctggtagggagtcagaaggagcccagcgaagtgccaacacctaagagacctcggggccgaccaaagggaagcaaaaacaagggtgctgccaagacccggaaaaccaccacaactccaggaaggaaaccaaggggcagacccaaaaaactggagaaggaggaagaggagggcatctcgcaggagtcctcggaggaggagcagtga

SEQ ID NO: 33 - DNA, 1875 bp, Homo sapiens
  misc_feature: NCIB Accession No. M23616
gctttttaagctcccctgagccggtgctgcgctcctctaattgggactccgagccggggctatttctgggctggcgcggctccaagaagatccgcatttgctaccagcggcggccgcgcggagccaggccggtcctcagcgcccagcacggctcccggcaacccggagcgcgcaccgcagccggcggccgagctcgcgcatcccagccatcactcttccacctgctccttagagaagggaagatgagtgagtcgagctcgaagtccagccagcccttggcctccaagcaggaaaaggacggcactgagaagcggggccggggcaggccgcgcaagcagcctccgaaggagcccagcgaagtgccaacacctaagagacctcggggccgaccaaagggaagcaaaaacaagggtgctgccaagacccggaaaaccaccacaactccaggaaggaaaccaaggggcagacccaaaaaactggagaaggaggaagaggagggcatctcgcaggagtcctcggaggaggagcagtgacccatgcgtgccgcctgctcctcactggaggagcagcttccttctgggactggacagctttgctccgctcccaccgcccccgccccttccccaggcccaccatcaccaccgcctctggccgccacccccatcttccacctgtgccctcaccaccacactacacagcacaccagccgctgcaggggctcccatgggcctgagtggggagcagttttcccctggcctcagttcacagctccccccgcccacccacgcatacacacatgccctcctggacaaggctaacatcccacttagccgcaccctgcacctgctgcgtccccactcccttggtggtggggacattgctctctgggcttttggtttgggggcgccctctctgctccttcactgttccctctggcttcccatagtggggcctgggagggttcccctggccttaaaaggggcccaagccatctcatcctggcacgccctactccactgccctggcacagcaggtgtggccaatggaggggggtgctggcccccaggattcccccagccaaactgtctttgtcaccacgtggggctcacttttcatccttccccaacttccctagtccccgtactaggttggacagcccccttcggctacaggaaggcaggaggggtgagtcccctactccctcttcactgtggccacagcccccttgccctccgcctgggatctgagtacatattgtggtgatggagatgcagtcacttattgtccaggtgaggcccaagagccctgtggccgcacctgaggtgggctggggctgctcccctaaccctactttcgttccgccactcagccatttccccctcctcagatggggcaccaataacaaggagctcaccctgcccgctcccaacccccctcctgctcctccctgccccccaaggttctgggttccatttttcctctgttcacaaactacctctggacagttgtgttgttttttgttcaatgttccattcttcgacatccgtcattgctgctgctaccagcgccaaatgttcatcctcattgcctcctgttctgcccacgatcccctcccccaagatactctttgtgggaagaggggctggggcatggcaggctgggtgaccgactaccccagtcccagggaaggtggccctgcccctaggatgctgcagcagagtgagcaagggggcccgaatcgaccataaagggtgtaggggccacctcctccccctgttctgttggggaggggtagccatgatttgtcccagcctggggctccctctctggtttcctatttgcagttacttgaataaaaaaaatatccttttctgg

SEQ ID NO: 34 - DNA, 291 bp, Homo sapiens
atgagtgagtcgagctcgaagtccagccagcccttggcctccaagcaggaaaaggacggcactgagaagcggggccggggcaggccgcgcaagcagcctccgaaggagcccagcgaagtgccaacacctaagagacctcggggccgaccaaagggaagcaaaaacaagggtgctgccaagacccggaaaaccaccacaactccaggaaggaaaccaaggggcagacccaaaaaactggagaaggaggaagaggagggcatctcgcaggagtcctcggaggaggagcagtga

SEQ ID NO: 35 - DNA, 4111 bp, Homo sapiens
acacaccacacacactcacactcacacacactcacacacactcatccccttgaatcttggggcaggaactcagaaaacttccagcccgggcagcgcgcgcttggtgcaagactcaggagctagcagcccgtccccctccgactctccggtgccgccgctgcctgctcccgccaccctaggaggcgcggtgccacccactactctgtcctctgcctgtgctccgtgcccgaccctatcccggcggagtctccccatcctcctttgctttccgactgcccaaggcactttcaatctcaatctcttctctctctctctctctctctctgtctctctctctctctctctctctctctctctcgcagggtggggggaagaggaggaggaattctttccccgcctaacatttcaagggacacaattcactccaagtctcttccctttccaagccgcttccgaagtgctcccggtgcccgcaactcctgatcccaacccgcgagaggagcctctgcgacctcaaagcctctcttccttctccctcgcttccctcctcctcttgctacctccacctccaccgccacctccacctccggcacccacccaccgccgccgccgccaccggcagcgcctcctcctctcctcctcctcctcccctcttctctttttggcagccgctggacgtccggtgttgatggtggcagcggcggcagcctaagcaacagcagccctcgcagcccgccagctcgcgctcgccccgccggcgtccccagccctatcacctcatctcccgaaaggtgctgggcagctccggggcggtcgaggcgaagcggctgcagcggcggtagcggcggcgggaggcaggatgagcgcacgcggtgagggcgcggggcagccgtccacttcagcccagggacaacctgccgccccagcgcctcagaagagaggacgcggccgccccaggaagcagcagcaagaaccaaccggtgagccctctcctaagagacccaggggaagacccaaaggcagcaaaaacaagagtccctctaaagcagctcaaaagaaagcagaagccactggagaaaaacggccaagaggcagacctaggaaatggccacaacaagttgttcagaagaagcctgctcaggaggaaactgaagagacatcctcacaagagtctgccgaagaggactagggggcgcaacgttcgatttctacctcagcagcagttggatcttttgaagggagaagacactgcagtgaccacttattctgtattgccatggtctttccactttcatctggggtggggtggggtggggtgggggaggggggggtggggtggggagaaatcacataaccttaaaaaggactatattaatcaccttctttgtaatcccttcacagtcccaggtttagtgaaaaactgctgtaaacacaggggacacagcttaacaatgcaacttttaattactgttttcttttttcttaacctactaatagtttgttgatctgataagcaagagtgggcgggtgagaaaaaccgaattgggtttagtcaatcactgcactgcatgcaaacaagaaacgtgtcacacttgtgacgtcgggcattcatataggaagaacgcggtgtgtaacactgtgtacacctcaaataccaccccaacccactccctgtagtgaatcctctgtttagaacaccaaagataaggactagatactactttctctttttcgtataatcttgtagacacttacttgatgatttttaactttttatttctaaatgagacgaaatgctgatgtatcctttcattcagctaacaaactagaaaaggttatgttcatttttcaaaaagggaagtaagcaaacaaatattgccaactcttctatttatggatatcacacatatcagcaggagtaataaatttactcacagcacttgttttcaggacaacacttcattttcaggaaatctacttcctacagagccaaaatgccatttagcaataaataacacttgtcagcctcagagcatttaaggaaactagacaagtaaaattatcctctttgtaatttaatgaaaaggtacaacagaataatgcatgatgaactcacctaattatgaggtgggaggagcgaaatctaaatttcttttgctatagttatacatcaatttaaaaagcaaaaaaaaaaaggggggggcaatctctctctgtgtctttctctctctctctccctctccctctctcttttcatgtgtatcagtttccatgaaagacctgaataccacttacctcaaattaagcatatgtgttacttcaagtaatacgttttgacataagatggttgaccaaggtgcttttcttcggcttgagttcaccatctcttcattcaaactgcacttttagccagagatgcaatatatccccactactcaatactacctctgaatgttacaacgaatttacagtctagtacttattacatgctgctatacacaagcaatgcaagaaaaaaacttactgggtaggtgattctaatcatctgcagttctttttgtacacttaattacagttaaagaagcaatctccttactgtgtttcagcatgactatgtatttttctatgtttttttaattaaaaatttttaaaatacttgtttcagcttctctgctagatttctacattaacttgaaaattttttaaccaagtcgctcctaggttcttaaggataattttcctcaatcacactacacatcacacaagatttgactgtaatatttaaatattaccctccaagtctgtacctcaaatgaattctttaaggagatggactaattgacttgcaaagacctacctccagacttcaaaaggaatgaacttgttacttgcagcattcatttgttttttcaatgtttgaaatagttcaaactgcagctaaccctagtcaaaactatttttgtaaaagacatttgatagaaaggaacacgtttttacatacttttgcaaaataagtaaataataaataaaataaagccaaccttcaaagaacttgaagctttgtaggtgagatgcaacaagccctgcttttgcataatgcaatcaaaaatatgtgtttttaagattagttgaatataagaaaatgcttgacaaatattttcatgtattttacacaaatgtgatttttgtaatatgtctcaaccagatttattttaaacgcttcttatgtagagtttttatgcctttctctcctagtgagtgtgctgactttttaacatggtattatcaactgggccaggaggtagtttctcatgacggcttttgtcagtatggcttttagtactgaagccaaatgaaactcaaaaccatctctcttccagctgcttcagggaggtagtttcaaaggccacatacctctctgagactggcagatcgctcactgttgtgaatcaccaaaggagctatggagagaattaaaactcaacattactgttaactgtgcgttaaataagcaaataaacagtggctcataaaaataaaagtcgcattccatatctttggatgggccttttagaaacctcattggccagctcataaaatggaagcaattgctcatgttggccaaacatggtgcaccgagtgatttccatctctggtaaagttacacttttatttcctgtatgttgtacaatcaaaacacactactacctcttaagtcccagtatacctcatttttcatactgaaaaaaaaagcttgtggccaatggaacagtaagaacatcataaaatttttatatatatagtttatttttgtgggagataaattttataggactgttctttgctgttgttggtcgcagctacataagactggacatttaacttttctaccatttctgcaagttaggtatgtttgcaggagaaaagtatcaagacgtttaactgcagttgactttctccctgttcctttgagtgtcttctaactttattctttgttctttatgtagaattgctgtctatgattgtactttgaatcgcttgcttgttgaaaatatttctctagtgtattatcactgtctgttctgcacaataaacataacagcctctgtga

SEQ ID NO: 36 - DNA, 330 bp, Homo sapiens
atgagcgcacgcggtgagggcgcggggcagccgtccacttcagcccagggacaacctgccgccccagcgcctcagaagagaggacgcggccgccccaggaagcagcagcaagaaccaaccggtgagccctctcctaagagacccaggggaagacccaaaggcagcaaaaacaagagtccctctaaagcagctcaaaagaaagcagaagccactggagaaaaacggccaagaggcagacctaggaaatggccacaacaagttgttcagaagaagcctgctcaggaggaaactgaagagacatcctcacaagagtctgccgaagaggactag

SEQ ID NO: 37 - DNA, 252 bp, Homo sapiens
atgagcgcacgcggtgagggcgcggggcagccgtccacttcagcccagggacaacctgccgccccagcgcctcagaagagaggacgcggccgccccaggaagcagcagcaagaaccaaccggtgagccctctcctaagagacccaggggaagacccaaaggcagcaaaaacaagagtccctctaaagcagctcaaaagaaagcagaagccactggagaaaaacggccaagaggcagacctaggaaatggtga

SEQ ID NO: 38 - DNA, 273 bp, Homo sapiens
atgagcgcacgcggtgagggcgcggggcagccgtccacttcagcccagggacaacctgccgccccagcgcctcagaagagaggacgcggccgccccaggaagcagcagcaagaaccaaccggtgagccctctcctaagagacccaggggaagacccaaaggcagcaaaaacaagagtccctctaaagcagctcaaaagaaagcagaagccactggagaaaaacggccaagaggcagacctaggaaatgggaggagttttacattgcagcttag

SEQ ID NO: 39 - DNA, 291 bp, Homo sapiens
atgagcgcacgcggtgagggcgcggggcagccgtccacttcagcccagggacaacctgccgccccagcgcctcagaagagaggacgcggccgccccaggaagcagcagcaagaaccaaccggtgagccctctcctaagagacccaggggaagacccaaaggcagcaaaaacaagagtccctctaaagcagctcaaaagaaagcagaagccactggagaaaaacggccaagaggcagacctaggaaatggcctactattgcactttgcacacactggataaacatctgctga

SEQ ID NO: 40 - DNA, 1207 bp, Homo sapiens
  misc_feature: NCIB Accession No. NM_002128
gagacagcgccggggcaagtgagagccggacgggcactgggcgactctgtgcctcgctgaggaaaaataactaaacatgggcaaaggagatcctaagaagccgagaggcaaaatgtcatcatatgcattttttgtgcaaacttgtcgggaggagcataagaagaagcacccagatgcttcagtcaacttctcagagttttctaagaagtgctcagagaggtggaagaccatgtctgctaaagagaaaggaaaatttgaagatatggcaaaggcggacaaggcccgttatgaaagagaaatgaaaacctatatccctcccaaaggggagacaaaaaagaagttcaaggatcccaatgcacccaagaggcctccttcggccttcttcctcttctgctctgagtatcgcccaaaaatcaaaggagaacatcctggcctgtccattggtgatgttgcgaagaaactgggagagatgtggaataacactgctgcagatgacaagcagccttatgaaaagaaggctgcgaagctgaaggaaaaatacgaaaaggatattgctgcatatcgagctaaaggaaagcctgatgcagcaaaaaagggagttgtcaaggctgaaaaaagcaagaaaaagaaggaagaggaggaagatgaggaagatgaagaggatgaggaggaggaggaagatgaagaagatgaagatgaagaagaagatgatgatgatgaataagttggttctagcgcagtttttttttcttgtctataaagcatttaacccccctgtacacaactcactccttttaaagaaaaaaattgaaatgtaaggctgtgtaagatttgtttttaaactgtacagtgtctttttttgtatagttaacacactaccgaatgtgtctttagatagccctgtcctggtggtattttcaatagccactaaccttgcctggtacagtatgggggttgtaaattggcatggaaatttaaagcaggttcttgttggtgcacagcacaaattagttatatatggggatggtagttttttcatcttcagttgtctctgatgcagcttatacgaaataattgttgttctgttaactgaataccactctgtaattgcaaaaaaaaaaaaagttgcagctgttttgttgacattctgaatgcttctaagtaaatacaattttttttattaaaaaaaaaaaaaaaaaaaaaaaaa

SEQ ID NO: 41 - DNA, 648 bp, Homo sapiens
atgggcaaaggagatcctaagaagccgagaggcaaaatgtcatcatatgcattttttgtgcaaacttgtcgggaggagcataagaagaagcacccagatgcttcagtcaacttctcagagttttctaagaagtgctcagagaggtggaagaccatgtctgctaaagagaaaggaaaatttgaagatatggcaaaggcggacaaggcccgttatgaaagagaaatgaaaacctatatccctcccaaaggggagacaaaaaagaagttcaaggatcccaatgcacccaagaggcctccttcggccttcttcctcttctgctctgagtatcgcccaaaaatcaaaggagaacatcctggcctgtccattggtgatgttgcgaagaaactgggagagatgtggaataacactgctgcagatgacaagcagccttatgaaaagaaggctgcgaagctgaaggaaaaatacgaaaaggatattgctgcatatcgagctaaaggaaagcctgatgcagcaaaaaagggagttgtcaaggctgaaaaaagcaagaaaaagaaggaagaggaggaagatgaggaagatgaagaggatgaggaggaggaggaagatgaagaagatgaagatgaagaagaagatgatgatgatgaataa

SEQ ID NO: 42 - DNA, 444 bp, Homo sapiens
atgagcgcacgcggtgagggcgcggggcagccgtccacttcagcccagggacaacctgccgccccagcgcctcagaagagaggacgcggccgccccaggaagcagcagcaagaaccaaccggtgagccctctcctaagagacccaggggaagacccaaaggcagcaaaaacaagagtccctctaaagcagctcaaaagaaagcagaagccactggagaaaaacggccaagaggcagacctaggaaatgggctggagtgcagtggtacaatctcggctcattgcaacctccacctcccaggttcaagcaattctcctgcctcaggctcctgagtagttgggattacaggcacccaccaccacacccagctaatttttgtatttttagtagagacagggtttcaccatgttggccaggctggtctcgaactcctgacctcaggtga

SEQ ID NO: 43 - DNA, 321 bp, Homo sapiens
atgagcgcacgcggtgagggcgcggggcagccgtccacttcagcccagggacaacctgccgccccagcgcctcagaagagaggacgcggccgccccaggaagcagcagcaagaaccaaccggtgagccctctcctaagagacccaggggaagacccaaaggcagcaaaaacaagagtccctctaaagcagctcaaaagaaagcagaagccactggagaaaaacggccaagaggcagacctaggaaatgggacaatctactaccaagaaccagctccaagaagaaaacatctctgggaaacagtaccaaaaggagtcactga

SEQ ID NO: 44 - DNA, 279 bp, Homo sapiens
atgagcgcacgcggtgagggcgcggggcagccgtccacttcagcccagggacaacctgccgccccagcgcctcagaagagaggacgcggccgccccaggaagcagcagcaagaaccaaccggtgagccctctcctaagagacccaggggaagacccaaaggcagcaaaaacaagagtccctctaaagcagctcaaaagaaagcagaagccactggagaaaaacggccaagaggcagacctaggaaatggtggttgctaatgaagagcccgtgctggtga

SEQ ID NO: 45 - DNA, 291 bp, Homo sapiens
atgagcgcacgcggtgagggcgcggggcagccgtccacttcagcccagggacaacctgccgccccagcgcctcagaagagaggacgcggccgccccaggaagcagcagcaagaaccaaccggtgagccctctcctaagagacccaggggaagacccaaaggcagcaaaaacaagagtccctctaaagcagctcaaaagaaagcagaagccactggagaaaaacggccaagaggcagacctaggaaatggccacaacaagttgttcagaagaagcctgctcagtattcctga

SEQ ID NO: 46 - DNA, 357 bp, Homo sapiens
atgagcgcacgcggtgagggcgcggggcagccgtccacttcagcccagggacaacctgccgccccagcgcctcagaagagaggacgcggccgccccaggaagcagcagcaagaaccaaccggtgagccctctcctaagagacccaggggaagacccaaaggcagcaaaaacaagagtccctctaaagcagctcaaaagaaagcagaagccactggagaaaaacggccaagaggcagacctaggaaatggccacaacaagttgttcagaagaagcctgctcaggtcaatgttgccttgcctgggaaggaccacccgggcaatcttatatatctactgttctctaaaaatgccacttag

SEQ ID NO: 47 - DNA, 288 bp, Homo sapiens
atgagcgcacgcggtgagggcgcggggcagccgtccacttcagcccagggacaacctgccgccccagcgcctcagaagagaggacgcggccgccccaggaagcagcagcaagaaccaaccggtgagccctctcctaagagacccaggggaagacccaaaggcagcaaaaacaagagtccctctaaagcagctcaaaagaaagcagaagccactggagaaaaacggccaagaggcagacctaggaaatggccacaacaagttgttcagaagaagcctgctcaggactga

SEQ ID NO: 48 - DNA, 33 bp, Homo sapiens
actgagaagcggggccggggcaggccgcgcaag

SEQ ID NO: 49 - DNA, 33 bp, Homo sapiens
acacctaagagacctcggggccgaccaaaggga

SEQ ID NO: 50 - DNA, 36 bp, Homo sapiens
actccaggaaggaaaccaaggggcagacccaaaaaa

SEQ ID NO: 51 - DNA, 33 bp, Homo sapiens; identical to SEQ ID NO: 48

SEQ ID NO: 52 - DNA, 33 bp, Homo sapiens; identical to SEQ ID NO: 49

SEQ ID NO: 53 - DNA, 36 bp, Homo sapiens; identical to SEQ ID NO: 50

SEQ ID NO: 54 - DNA, 33 bp, Homo sapiens
cctcagaagagaggacgcggccgccccaggaag

SEQ ID NO: 55 - DNA, 33 bp, Homo sapiens
tctcctaagagacccaggggaagacccaaaggc

SEQ ID NO: 56 - DNA, 63 bp, Homo sapiens
actggagaaaaacggccaagaggcagacctaggaaatggccacaacaagttgttcagaagaag

SEQ ID NO: 57 - DNA, 234 bp, Homo sapiens
cctaagaagccgagaggcaaaatgtcatcatatgcattttttgtgcaaacttgtcgggaggagcataagaagaagcacccagatgcttcagtcaacttctcagagttttctaagaagtgctcagagaggtggaaggtaagagggcttaaaacatgctaacaaggtaattaaaagacagtttccaattgaggatgcaaaaaaaagcctagttggcattctcgtagtgggacgcta

SEQ ID NO: 58 - DNA, 213 bp, Homo sapiens
ccgagaggcaaaatgtcatcatatgcattttttgtgcaaacttgtcgggaggagcataagaagaagcacccagatgcttcagtcaacttctcagagttttctaagaagtgctcagagaggtggaagaccatgtctgctaaagagaaaggaaaatttgaagatatggcaaaggcggacaaggcccgttatgaaagagaaatgaaaacctatatc

SEQ ID NO: 59 - DNA, 219 bp, Homo sapiens
cctaagaagccgagaggcaaaatgtcatcatatgcattttttgtgcaaacttgtcgggaggagcataagaagaagcacccagatgcttcagtcaacttctcagagttttctaagaagtgctcagagaggtggaagaccatgtctgctaaagagaaaggaaaatttgaagatatggcaaaggcggacaaggcccgttatgaaagagaaatgaaaacctat

SEQ ID NO: 60 - DNA, 225 bp, Homo sapiens
cccaatgcacccaagaggcctccttcggccttcttcctcttctgctctgagtatcgcccaaaaatcaaaggagaacatcctggcctgtccattggtgatgttgcgaagaaactgggagagatgtggaataacactgctgcagatgacaagcagccttatgaaaagaaggctgcgaagctgaaggaaaaatacgaaaaggatattgctgcatatcgagctaaagga

SEQ ID NO: 61 - DNA, 207 bp, Homo sapiens
cccaagaggcctccttcggccttcttcctcttctgctctgagtatcgcccaaaaatcaaaggagaacatcctggcctgtccattggtgatgttgcgaagaaactgggagagatgtggaataacactgctgcagatgacaagcagccttatgaaaagaaggctgcgaagctgaaggaaaaatacgaaaaggatattgctgcatatcga

SEQ ID NO: 62 - DNA, 147 bp, Homo sapiens
cccaagaggcctccttcggccttcttcctcttctgctctgagtatcgcccaaaaatcaaaggagaacatcctggcctgtccattggtgatgttgcgaagaaactgggagagatgtggaataacactgctgcagatgacaagcagcct

SEQ ID NO: 63 - DNA, 546 bp, Homo sapiens
gaggagcataagaagaagaacccagatgcttcagtcaagttctcagagtttttaaagaagtgctcagagacatggaagaccatttttgctaaagagaaaggaaaatttgaagatatggcaaaggcggacaaggcccattatgaaagagaaatgaaaacctatatccctcctaaaggggagaaaaaaaagaagttcaaggatcccaatgcacccaagaggcctcctttggcctttttcctgttctgctctgagtatcgcccaaaaatcaaaggagaacatcctggcctgtccattgatgatgttgtgaagaaactggcagggatgtggaataacaccgctgcagctgacaagcagttttatgaaaagaaggctgcaaagctgaaggaaaaatacaaaaaggatattgctgcatatcgagctaaaggaaagcctaattcagcaaaaaagagagttgtcaaggctgaaaaaagcaagaaaaagaaggaagaggaagaagatgaagaggatgaacaagaggaggaaaatgaagaagatgatgataaataa

SEQ ID NO: 64 - DNA, 678 bp, Homo sapiens
atgagcgcacgcggtgagggcgcggggcagccgtccacttcagcccagggacaacctgccgccccagcgcctcagaagagaggacgcggccgccccaggaagcagcagcaagaaccaaccggtgagccctctcctaagagacccaggggaagacccaaaggcagcaaaaacaagagtccctctaaagcagctcaaaagaaagcagaagccactggagaaaaacggccaagaggcagacctaggaaatggaatactctggagcagtgcaatgtgtgttccaagcccatcatggagcggattctccgagccaccgggaaggcctatcatcctcactgtttcacctgcgtgatgtgccaccgcagcctggatgggatcccattcactgtggatgctggcgggctcattcactgcattgaggacttccacaagaaatttgccccgcggtgttctgtgtgcaaggagcctattatgccagccccgggccaggaggagactgtccgtattgtggctttggatcgagatttccatgttcactgctaccgatgcgaggattgcggtggtctcctgtctgaaggagataaccaaggctgctaccccttggatgggcacatcctctgcaagacctgcaactctgcccgcatcagggtgttgaccgccaaggcgagcactgacctttag

(19) United States
(12) Patent Application Publication
(10) Pub. No.: US 2007/0154529 A1
(43) Pub. Date: Jul. 5, 2007

(54) Uses of dna binding proteins

(75) Inventor: Jorn Bullerdiek, Bremen (DE)

Correspondence Address: PROSKAUER ROSE LLP, 1001 PENNSYLVANIA AVE, N.W.,, SUITE 400 SOUTH, WASHINGTON, DC, 20004, US

(73) Assignee: ALCEDO BIOTECH GMBH, BREMEN (DE)

(21) Appl. No.: 10/541,354

(22) PCT Filed: Jan. 5, 2004
(86) PCT No.: PCT/EP04/00030

§ 371 Date: Jun. 21, 2006

(30) Foreign Application Priority Data
Jan. 3, 2003 (DE) 103 00 023.2
Mar. 7, 2003 (DE) 103 10 160.8
Aug. 10, 2003 (DE) 103 36 642.3
Oct. 8, 2003 (DE) 103 46 614.2

(51) Int. Cl. A61K 48/00; A61L 15/00

(52) U.S. Cl. 424/445; 514/44

(57) ABSTRACT

The invention relates to the use, especially in vitro, of one or more nucleic acids, the transcription product(s) thereof and/or the translation product(s) thereof for a process. Said process is selected from the group including angiogenesis, neovascularization, transmyocardial revascularization, wound healing, wound bed angiogenesis, epithelization and healing in of dental and bone implants. The nucleic acid(s) is/are selected from the group including the genes for high mobility group proteins.

The present invention is related to the use of nucleic acids, the transcription products thereof and/or translation products thereof for angiogenesis, neovascularisation, transmyocardial revascularisation, dedifferentiation of cells and/or reprogramming of cells, for tissue regeneration, for influencing tissue aging and for wound healing, methods for angiogenesis, vascularization, neovascularization and transmyocardial revascularization as well as for tissue regeneration, wound healing, for influencing tissue aging, methods for vascularization, in particular upon cardiac infarction and healing of tooth and bone implants, methods for the regeneration of tissue, methods for dedifferentiation and/or reprogramming of cells, carrier material comprising such a nucleic acid, the transcription product thereof and/or the translation products thereof as well as a wound cover material comprising a basic cover material and nucleic acid, the transcription product thereof and/or translation product thereof.

The various aspects of the present invention have in common that the processes involved therein seem to be related to the (de)differentiation of cells which are suitable for use in various medical applications. In particular, this applies to the vasculature of vertebrates, preferably mammals and human beings, and the skin thereof.

The vasculature of humans consists of arteries, arterioles, capillaries, terminal vascular beds, venules and veins. Arteries are vessels providing blood flow from the heart, whereby two types can be distinguished: arteries of the muscular type and arteries of the elastic type, whereby the latter are arteries close to the heart. Arteries generally consist of a tunica interna, which is also referred to as intima, with a single-layered endothelium facing the lumen, the loose stratum subendotheliale of the connective tissue type, and the membrana elastica interna, which is well developed in the muscular type, additionally of the tunica media which, in the muscular type, consists of densely packed layers of smooth muscle cells arranged in a circular or helical manner, and of fine elastic collagene fibres, whereas in the arteries of the elastic type consists of numerous elastic membranes having fenastrae and embedded smooth muscle cells as well as collagen fibres and the tunica externa consisting of collagenous connective tissue and elastic fibres and nutritional vessels as well as vessel nerves. The membrana elastica externa can be formed between the tunica media and the tunica externa.

The arteries comprise as last vessel portions the arterioles consisting of an endothelium, a lattice fibre net and a single-layered contiguous smooth muscle cell layer, whereby the membrana elastica interna which is still present in arteries, is absent, so that there are myoendothelial contacts.

The arterioles transform into the capillaries which are small vessels having a diameter of about six to about twenty to thirty μm. The wall of the capillaries consists of endothelium which is based on a basal membrane surrounded by lattice fibres. This basal membrane is externally covered by branched cells, the so-called pericytes. The pericytes are most likely involved in the material transfer between the capillary blood and the tissue.

The blood capillaries' then transform into the venules and finally into the veins, i.e. blood vessels providing blood flow to the heart. The wall of typical veins comprises a tunica interna having numerous elastic fibres, however, no membrana elastica interna, furthermore a tunica media with loosely arranged bundles of smooth muscles as well as a tunica externa. In contrast to arteries the bounders of these layers are unsharp in histological preparations.

The part of the vasculature consisting of arterioles, capillaries and venules defining the microciruclation of the blood is referred to as terminal vascular bed and is a neutral portion in terms of hemodynamics between arterial influx and venous efflux of blood, which is thus the turning point of the circulation system. In this portion material and gas exchange between blood and tissue as well as the maintenance of the thermal and ionic environment occurs.

The skin is an important organ of the mammal body, in particular also of the human body. The skin consists of the skin in the narrower sense, i.e. the dermis, which is followed by the epidermis and the cutis, which is also referred to as corium, followed by appendages such as hair, nails and glands, and the hypodermis or subcutis as elements of the skin in the broader sense. The functions of the skin are extremely diverse. It serves as a mechanical barrier against the environment, as heat protection organ which is effective in blood distribution and temperature control due to its excessive blood flow, but also through its insulation effect of the hair and of the fat pad as well as by evaporation of sweat due to which it is also involved in the control of water metabolism, as protective organ against bacteria due to the acidic coverage as well as against radiation because of pigment formation, and energy storage due to the fat storage. Additionally, the skin is an important sensory organ due to the end organs embedded therein. Additionally, it is an immune organ having different defence functions. Due to this the skin attracts a lot of attention, particularly in connection with wound healing and skin aging.

Wound healing is a dynamic process involving complex interactions between cells, extracellular matrix, plasma membranes and a controlled angiogenesis which is coordinated through a variety of cytokines and growth factors. Independent of the kind of wound and the extent of tissue loss, wound healing can be grouped into timely overlapping phases such as the inflammatory and exudative phase, respectively, the proliferative phase as well as the differentiation and re-organisation phase. This grouping is in principle based on morphological changes in the course of the repair processes without reflecting the true complexity of the processes.

The process of wound healing can be quantitatively divided into primary and secondary wound healing, whereby, in order to reflect the therapeutic problems which might arise from the extent and the kind of tissue damage, it can be further divided into a delayed primary healing as well as a chronic wound course. Primary wound healing, for example, exists if there are smooth, tightly arranged wound surfaces of a cut without significant loss of tissue and without any deposition of xenoliths in a tissue which is well vascularized. Primary wound healing usually happens in connection with surgical wounds and with occasional wounds caused by sharp-edged objects. If one has, due to the way the wound was caused, to take into consideration an infection, there will be delayed primary healing. If there is an infection, the wound is categorised as healing by second intention. Healing by second intention exists in case of bigger defects in connection with which a granulation tissue has to be built up, or if the infection does not allow for an immediate closing of the wound edges. If the healing is not completed within eight weeks, this is referred to as a chronic course of healing. A chronic wound can come into being in any wound healing phase and usually results from a progressing destruction if tissue due to tissue diseases of different origins, local pressure damage, radiation damage or tumors.

A further categorization of wound healing can be based on a discrimination between acute wounds and chronic wounds. Acute wounds range from acute traumatic wounds to complex traumatic defects, thermal and chemical wounds/burns and incisions/surgery wounds.

In the case of acute traumatic wounds the primary closing of the wound is made by suture, clips or approximation stripes, provided that the wound edges can be adapted without tension and after an optional wound excision. In case of wounds which potentially exhibit an infection, the wound is, at first, kept open by sterile humid dressings until an infection can be excluded. In secondary healing and complex wounds the closing of the wound is more complex.

In case of thermal and chemical wounds, i.e. wounds generated by heat, cold or tissue damaging radiation, acids, or bases, the treatment is made in accordance with the damaging pattern. For example, heavily burned patients undergo first a necrectomy with subsequent surgical replacement by a skin transplant. If the wound cannot be transplanted or if there are not sufficient donor sites available due to the extent of the burn, so-called allo or xenotransplants are used. If there are sufficient donor areas, one can use permanent autologous skin transplants. A particular form is the autologous keratinocyte transplantation.

Chronic wounds are secondary healing wounds which, despite causal and appropriate local therapy, do not heal within eight weeks. Although chronic wounds can result an acute wound at any time from, chronic wounds predominantly represent the last stadium of a progressing tissue damage which is caused by venous, arterial or metabolism-caused vascular diseases, pressure damage, radiation damage and tumors. The different types of chronic wounds are caused by different pathologies, whereby the wounds, speaking in terms of biochemistry, are regarded as similar. The local factors which have an impact on wound healing, are, among others, xenoliths, ischaemia, repeated traumata and infection. Additionally, systemic factors such as old age, undernutrition or malnutrition, diabetes as well as renal diseases may have an impact on wound healing. The economically most relevant chronic wound healing disorders are, among others, ulcus cruris venosum, ulcus cruris arteriosum, diabetic ulcer, decubital ulcer and chronic posttraumatic wounds.

Apart from acute and chronic wounds skin aging is an essential change of the skin which can be divided into aging caused by time and aging caused by environmental factors. The term aging by time refers to the changes arising from usual aging processes of the skin which result in a thinning of the skin layers and in a decrease of the function of the skin glands. This causes, at an increasing age, a thin, dry and finely wrinkled skin. Age-caused microwrinkles and wrinkles result from a decrease and loss, respectively, of collagen and of elastic fibers in the corium. Additionally, the integrity of aged skin is more easily disturbed and it regenerates slower so that the organism is exposed to an increased infection risk. An age-caused retardation and decrease in cell regeneration is influenced by, among others, hormonal changes and hereditary factors. However, environmental factors have a significant impact on this aging process which can be accelerated and promoted.

In aging caused by environmental factors the extent of lifelong UV radiation is important; that is why it is also called “photoaging”. However, also other factors promote these aging processes such as decreased blood flow in the skin due to nicotine abuse. While the UV-B portion of the sunlight primarily triggers damages in the cells of the epidermis and thus results in precursor forms of skin cancer (so-called actinic keratosis) and skin cancer (basalioma, squamous carcinoma, melanoma), UV-A radiation reaches the corium and destroys the connective tissue of the skin (elastosis). This results in the face, but also in the neck, in a loose, strongly wrinkled, folded skin. A further result of permanent UV exposure are so-called senile speckles which preferably occur at light-exposed areas in the face and on the back of the hands. In contrast to this hyperpigmentation light may also result in a loss of pigmentation (hypomelanosis guttata). These phenomena are referred to as chronic light damage which is regarded as an irreversible process.

The treatment of the various wounds can basically be categorised in a passive and an active wound therapy. As a particular form also skin replacement methods maybe used. Inactive, textile dressings which serve as a mere cover material so as to provide protection against infection, are used in passive wound therapy. Interactive wound coverage is, in contrast to the inactive dressings, frequently intended to create a humid wound environment thus promoting the healing process. In connection therewith, among others, hydrocolloids, hydrogels, hydropolymers and foam dressings as well as calcium alginates are used. The disadvantage of such passive wound therapy is that the dressing does not promote the active healing of problematic wounds, in particular chronic wounds which are frequently regarded as being therapy resistant.

In the prior art, there are different growth factors described which can be used in angiogenesis and/or active wound therapy and which target individual target molecules of the wound healing process. These are, among others, VEGF, transforming growth factor beta (TGF beta), platelet derived growth factor (PDGF), fibroblast growth factor (FGF), interleukin 1 beta, granulocyte macrophage colony stimulating factor (GM-CF) and blood-clotting factor XIII. However, the hopes put into these compounds have not come true which, at least partially, results from the complexity of the wound healing process.

In connection with skin substitute methods for the coverage of wounds, it is distinguished between temporary and permanent skin substitutes. The temporary skin substitute can either be of allogen biological origin, such as, for example, foreign skin such as decellularised human cutis from dead bodies in combination with sheets of ceratinocytes or split-thickness skin, can be of xenogenous biological origin, such as equine collagen fibers or bovine collagen sponges, or a combination of synthetic and biological material such as sheets of silicone or nylone combined with collagen matrices and/or fibroblasts. The permanent skin substitute may be an autologous skin transplant or be based on cell cultures.

Due to the different physiological processes involved in wound healing on the one hand and skin aging on the other hand, different therapeutic and cosmetic approaches, respectively, are practised in fighting skin aging by means of so-called anti-aging-products. According to a study performed by Stiftung Warentest (test Spezial Kosmetik 2002, page 17-19, special edition), most of the preparations available on the market are not effective or are only little effective, in particular in terms of smoothening of wrinkles. In connection with anti-aging products only those strategies are effective which are targeting a slowing down of the aging processes. Accordingly, vitamines and antioxidants are used in order to protect the skin against external, environmental factors such as UV-B radiation or air pollution. Apart from that, wrinkles and other little skin damages are treated by cosmetic surgery. This, however, requires a significant involvement of instrumentation. A further technique which is currently used for removing wrinkles is the injection of botulinum toxin. Botulinum toxin results in an intoxication and thus paralysis of muscle cells in the area of the wrinkles where it is injected thus lifting the skin. Apart from unexplained side effects a further disadvantage of the therapy is that, upon practicing, subsequently in about 5% of the patients the therapy is no longer effective due to the formation of neutralising antibodies.

The problem underlying the present invention is to provide means in order to promote or inhibit angiogenesis or neovascularization, or to promote transmyocardial revascularization and thus allowing the treatment of diseases related thereto. In a further aspect the problem is to provide means for promoting and initiating, respectively, wound healing.

The problem underlying the present invention is also to provide means in order to transfer cells, particularly mesenchymal cells, but also epithelial cells, in a condition which allows them to differentiate, optionally to dedifferentiate and/or to grow. In a further aspect the problem underlying the present invention is to provide a means for promoting and inhibiting, respectively, wound healing. Finally, the problem underlying the present invention is to provide means for fighting skin aging.

In a first aspect of the present invention the problem is solved by the use, particularly in vitro use, of one or several nucleic acid(s), the transcription product(s) thereof and/or the translation product(s) thereof in a process, whereby the process is selected from the group comprising angiogenesis, neovascularization, transmyocardial revascularization, wound healing, angiogenesis following wounding, epithelialization and healing of tooth and bone implants,

whereby the nucleic acid(s) is/are selected from the group comprising genes for the high mobility group proteins.

In a second aspect of the present invention the problem is solved by the Use of one or several nucleic acid(s), the transcription product(s) thereof and/or the translation product(s) thereof for the manufacture of a medicament for the prevention and/or treatment of a disease, whereby the disease is selected from the group which is related to lacking or excessive angiogenesis or neovascularization, or wound healing, or requires transmyocardial revascularization,

whereby the nucleic acid(s) is/are selected from the group comprising genes for the high mobility group proteins.

In a first aspect of the present invention which is preferably an embodiment of the first and second aspect, the problem is solved by the use of one or several nucleic acid(s), the transcription product(s) thereof and/or the translation product(s) thereof for the manufacture of a medicament for the prevention and/or treatment of a disease, characterised in that the disease is selected from the group comprising diabetic retinopathy, proliferative retinopathia diabetica, diabetic nephropathy, macular degeneration, arthritis, endometriosis, pannus, histiocytosis, psoriasis, rosacea, small varicose veins, eruptive hemangioma, tumor diseases, cavernoma, lip angioma, haemangiosarcoma, haemorrhoids, artherosclerosis, angina pectoris, ischemia, infarction, basalioma, squamous carcinoma, melanoma, Kaposi's sarcoma, tumors, gestosis, infertility, acute traumatic wounds, thermal wounds, chemical wounds, surgical wounds and chronic wounds.

In an embodiment of the first, second and third aspect the chronic wound is selected from the group comprising decubitus, ulcus cruris, ulcus cruris venosum, ulcus cruris arteriosum, diabetic ulcus, decubital ulcer, chronic post-traumatic wound and diabetic foot ulcers.

In an embodiment of the first, second and third aspect the high mobility group protein is selected from the group comprising the HMGA family, the HMGB family and the HMGN family.

In an embodiment of the first, second and third aspect the high mobility group protein is selected from the HMGB family.

In a preferred embodiment of the first, second and third aspect the high mobility group protein is selected from the group comprising HMGB1, HMGB2 and HMGB3.

In a more preferred embodiment of the first, second and third aspect the high mobility group protein is HMGB1.

In an embodiment of the first, second and third aspect the high mobility group protein is selected from the HMGA family.

In a preferred embodiment of the first, second and third aspect the high mobility group protein is selected from the group comprising HMGA1a, HMGA1b, HMGA1c and HMGA2.

In a more preferred embodiment of the first, second and third aspect the high mobility group protein is HMGA1a.

In an embodiment of the first, second and third aspect one high mobility group protein is selected from the HMGA family, and a second high mobility group protein is selected from the HMGB family, whereby the protein of the HMGA family is preferably HMGA1a and the protein of the HMGB family is preferably HMGB1.

In an embodiment of the first, second and third aspect VEGF and/or a nucleic acid coding therefor, is additionally used.

In a fourth aspect of the present invention the problem is solved by a method for affecting angiogenesis or neovascularization or wound healing of a tissue comprising the following steps:
a) providing a tissue or a part thereof,
b) adding one or several nucleic acid(s), transcription product(s) thereof and/or translation product(s) and
c) incubating the tissue with the nucleic acid(s), the transcription product(s) thereof and/or the translation product(s) thereof,
   whereby the nucleic acid(s) is/are selected from the group comprising the genes for the high mobility group proteins, and, optionally,
d) obtaining or recovering the tissue or an intermediate thereof.

In an embodiment of the fourth aspect the tissue or a part thereof is incubated with VEGF and/or a nucleic acid coding therefor.

In an embodiment of the fourth aspect the method is an in vitro method.

In an embodiment of the fourth aspect the tissue is an explanted tissue or an in vitro cultured tissue.

In an embodiment of the fourth aspect the nucleic acid(s), the transcription product(s) thereof and the translation product(s) thereof is/are such as described in any of the preceding aspects and embodiments.

In an embodiment of the fourth aspect two or more of the HMGB proteins or of the nucleic acid(s) coding therefor are used, whereby preferably one high mobility group protein is selected from the HMGA family and a second high mobility group protein is selected from the HMGB family, whereby the protein of the HMGA family is preferably HMGA1a, and the protein from the HMGB family is preferably HMGB 1.

In an embodiment of the first, second, third and fourth aspect, in addition to the nucleic acid(s), the transcription product(s) thereof and/or the translation product(s) thereof, whereby the nucleic acid is selected from the group comprising the genes for the high mobility group protein, a nucleic acid, the transcription product thereof or the translation product thereof, is used, whereby the nucleic acid is selected from the group comprising the gene for vascular endothelial growth factor.

In a fifth aspect of the present invention the problem is solved by a pharmaceutical formulation comprising one or several nucleic acid(s), the transcription product(s) thereof and/or the translation product(s) thereof, as described herein, and a pharmaceutically acceptable carrier.

In a sixth aspect of the present invention the problem is solved by a carrier material comprising one or several nucleic acid(s), the transcription product(s) thereof and/or the translation product(s) thereof, whereby the nucleic acid(s), the transcription product(s) thereof and/or the translation product(s) thereof is/are such as described in any of the preceding claims.

In an embodiment of the sixth aspect the carrier material the carrier material consists of a material which is selected from the group comprising cellulose, agarose, collagen, silicone, silicon, plastics, gels, hydrogels, matrices based on fibrin, man-made continuous filament yarn, hydrocolloids, lipocolloids, polyurethane, polyurethane resins, plaster, synthetic biomaterials, thermoplastic plastics, zinc glue, polyester foam, polyisobutylene, buffer, stabilizers, bacteriostatics and moisturizer.

In an embodiment of the sixth aspect the carrier material is serving as an implant or for wound healing.

In a seventh aspect of the present invention the problem is solved by a wound cover material comprising a basic cover material and one or several nucleic acid(s), the transcription product(s) thereof and/or the translation product(s) thereof, whereby the nucleic acid(s), the transcription product(s) thereof and/or the translation product(s) thereof is/are such as described in any of the preceding claims.

In an embodiment of the seventh aspect the cover material is selected from the group comprising hydrocolloidal dressings, calcium alginate dressings, compresses and overlays of activated carbon, overlays of foamed plastic, film dressings, transparent dressings, silicone foam dressings, fleece overlays, hydrocellular dressings, hydroselective wound overlays, absorbing wound pads, spray dressings, gauze of man-made continuous filaments, cotton gauze, paraffin gauze, silver coated wound dressings and hydropolymer/foam dressings.

In an eighth aspect of the present invention the problem is solved by a formulation comprising one or several nucleic acid(s), the transcription product(s) thereof and/or the translation product(s) thereof, whereby the nucleic acid(s), the transcription product(s) thereof and/or the translation product(s) thereof is/are such as described in any of the preceding claims, and a carrier phase, whereby the carrier phase is preferably selected from the group comprising creams, fatty ointments, emulsions (oil in water (O/W); water in oil (W/O); water in oil in water (W/O/W)); microemulsions, modified emulsions, nanoparticles/nanoemulsions, liposomes, hydrodispersion gels (hydrogels, alcoholic gels, lipogels, tenside gels), gel-creams, lotions, oils/oil baths and sprays.

In a ninth aspect of the present invention the problem is solved by a method for the screening of a compound for promoting and/or inhibiting a process, whereby the process is selected from the group comprising angiogenesis, neovascularization, transmyocardial revascularization and wound healing, comprising the following steps:
a) providing a test system for the process;
b) providing a candidate compound; and
c) testing the candidate compound and determining the reaction caused by the candidate compound in the test system.

In a tenth aspect of the present invention the problem is solved by a method for the screening of a compound for promoting and/or inhibiting a process, whereby the process is selected from the group comprising angiogenesis, neovascularization, transmyocardial revascularization and wound healing, comprising the following steps:
a) providing a test system for the process;
b) providing a reference compound;
c) testing the reference compound in the test system and determining the reaction caused by the reference compound in the test system;
d) providing a candidate compound;
e) testing the candidate compound in the test system and determining the reaction caused by the candidate compound in the test system; and
f) comparing the reaction of the reference compound in the test system to the reaction of the candidate compound in the test system.

In an eleventh aspect of the present invention the problem is solved by a method for the screening of a compound for the promotion and/or inhibition of a process, whereby the process is selected from the group comprising angiogenesis, neovascularization, transmyocardial vascularization and wound healing, comprising the following steps:
a) providing a test system for the process;
b) providing a reference compound, whereby the reference compound has a marker;
c) testing the reference compound in the test system and determining the reaction caused by the reference compound in the test system;
d) providing the candidate compound; and
e) testing the candidate compound in the test system, whereby the test system comprises the reference compound, and determining the reaction of the test system, whereby the amount of released reference compound and/or released marker of the reference compound is determined.

In a twelfth aspect of the present invention the problem is solved by a method for the screening of a compound for the promotion and/or inhibition of a process, whereby the process is selected from the group comprising angiogenesis, neovascularization, transmyocardial vascularization and wound healing, comprising the following steps:
a) providing a test system for the process;
b) providing a candidate compound, whereby the candidate compound has a marker;
c) testing the candidate compound in the test system and determining the reaction caused by the candidate compound in the test system;
d) providing a reference compound; and
e) testing the reference compound in a test system, whereby the test system comprises a candidate compound, and determining the reaction of the test system, whereby the amount of released candidate compound and/or of released marker of the candidate compound is determined.

In an embodiment of the ninth, tenth, eleventh and twelfth aspect the test system is an in vitro test system or a in vivo test system.

In an embodiment of the ninth, tenth, eleventh and twelfth aspect the reaction of the reference compound and/or of the candidate compound is a promotion of the process, and whereby preferably the candidate compound is a compound for promoting the process if the reaction of the candidate compound in the test system is identical or more pronounced than the reaction of the reference compound.

In an embodiment of the ninth, tenth, eleventh and twelfth aspect the reaction of the reference compound and/or the candidate compound is an inhibition of the process, and whereby preferably the candidate compound is a compound for inhibiting the process, if the reaction of the test system caused by the candidate compound is a reaction which is less pronounced than the one caused by the reference compound in the test system.

In an embodiment of the ninth, tenth, eleventh and twelfth aspect the reference compound comprises one or several nucleic acid(s), the transcription product(s) thereof and/or the translation product(s) thereof, whereby the nucleic acid is selected from the group comprising genes for high mobility group proteins, preferably as defined in any of the preceding claims.

In an embodiment of the ninth, tenth, eleventh and twelfth aspect the process is the inhibition of angiogenesis.

In a thirteenth aspect of the present invention the problem is solved by the use of a method according to any of the ninth to the twelfth aspect for the screening of a compound for the treatment and/or prevention of a disease, whereby the test system provided is a test system for the respective disease.

In an embodiment of the thirteenth aspect the disease is selected from the group comprising diseases which require the promotion or inhibition of angiogenesis or neovascularization, or transmyocardial revascularization or wound healing.

In a preferred embodiment of the thirteenth aspect the disease is selected from the group comprising diabetic retinopathy, proliferative retinopathia diabetica, diabetic nephropathy, macular degeneration, arthritis, endometriosis, pannus, histiocytosis, psoriasis, rosacea, small varicose veins, eruptive hemangioma, tumor diseases, cavernoma, lip angioma, haemangiosarcoma, haemorrhoids, artherosclerosis, angina pectoris, ischemia, infarction, basalioma, squamous carcinoma, melanoma, Kaposi's sarcoma, tumors, gestosis, infertility, acute traumatic wounds, thermal wounds, chemical wounds, surgical wounds and chronic wounds.

In an embodiment of the thirteenth aspect the disease is a tumor disease, whereby preferably the tumor diseases comprise necrotic cells, preferably necrotic tumor cells.

In a fourteenth aspect of the present invention the problem is solved by a compound obtainable by a method according to any of the ninth to twelfth aspect.

In a fifteenth aspect of the present invention the problem is solved by the use of a compound according to the fourteenth aspect for the manufacture of a medicament, preferably for the treatment and/or inhibition of a disease, as defined herein.

In a sixteenth aspect of the present invention the problem is solved by the use, particularly in vitro use, of a nucleic acid, the transcription product thereof and/or the translation product thereof, for a process, whereby the process is selected from the group comprising tissue regeneration, repair of DNA damages, wound healing, cell mobility, angiogenesis in the wound area, epithelialization, tissue aging, prevention of tissue aging, rejuvenation of tissue, vascularization after cardiac infarction and healing of tooth and bone implants,

whereby the nucleic acid is selected from the group comprising genes for basic DNA binding proteins.

In a seventeenth aspect of the present invention the problem is solved by the use, particularly in vitro use, of a nucleic acid, the transcription product thereof and/or the translation product thereof, for a process, whereby the process is selected from the group comprising dedifferentiation of cells and re-programming of cells, for tissue build-up and/or tissue regeneration, in particular based on dedifferentiation and/or differentiation of the tissue to be build up or to be regenerated,

whereby the nucleic acid is selected from the group comprising genes for basic DNA binding proteins.

In an eighteenth aspect of the present invention the problem is solved by the use of a nucleic acid, the transcription product thereof and/or the translation product thereof, for the manufacture of a medicament for prevention and/or treatment of a disease, whereby the disease is selected from the group comprising diseases which require the repair DNA damages, diseases which require tissue regeneration, diseases which require wound healing, diseases which go along with tissue aging, diseases which require tooth and bone implants, diseases which go along with tissue aging, wound healing disorders, skin diseases, xeroderma pigmentosum, leather skin, skin cancer, skin cancer after burn, skin aging after burn, burn and cardiac infarction,

whereby the nucleic acid is selected from the group comprising genes for basic DNA-binding proteins.

In a nineteenth aspect of the present invention the problem is solved by the use of a nucleic acid, the transcription product thereof and/or the translation product thereof, for the manufacture of a cosmetic product, preferably a cosmetic product for tissue regeneration, wound healing, prevention of leather skin, prevention of skin cancer, in particular skin cancer after sun burn, skin aging, in particular skin aging after sun burn, tissue aging inhibition and/or tissue juvenation,

whereby the nucleic acid is selected from the group comprising genes for basic DNA-proteins.

In a twentieth aspect of the present invention the problem is solved by the use of a nucleic acid, the transcription product thereof and/or the translation product thereof for the manufacture of a medicament for the prevention and/or treatment of a disease, whereby the disease is selected from the group comprising skin diseases, xeroderma pigmentosum, leather skin, skin cancer, skin cancer after sun burn, sun burn, acute wounds and chronic wounds,

whereby the nucleic acid is selected from the group comprising genes for basic DNA-binding proteins.

In an embodiment of the twentieth aspect the acute wound is selected from the group comprising acute traumatic wounds, thermal wounds, chemical wounds and surgical wounds.

In an embodiment of the twentieth aspect the chronic wound is selected from the group comprising decubitus, ulcus cruris, ulcus cruris venosum, ulcus cruris arteriosum, diabetic ulcus, decubital ulcer, chronic post-traumatic wounds and diabetic foot ulcer.

In an embodiment of the sixteenth to the twentieth aspect the basic DNA-binding protein is selected from the group comprising high mobility group proteins.

In an embodiment of the sixteenth to the twentieth aspect the high mobility group protein is selected from the group comprising HMGA, HMGB and HMGN.

In an embodiment of the sixteenth to the twentieth aspect the high mobility group protein is a protein of the HMGA family.

In a preferred embodiment of the sixteenth to the twentieth aspect the protein is selected from the group comprising HMGA1a, HMGA1b and HMGA2.

In an embodiment of the sixteenth to the twentieth aspect the nucleic acid is selected from the group comprising nucleic acids according to SEQ. ID. NO. 31 to SEQ. ID. NO. 64 and respective derivatives.

In an embodiment of the sixteenth to the twentieth aspect the translation product is selected from the group comprising polypeptides having a sequence according to SEQ. ID. NO. 1 to SEQ. ID. NO. 30 and the respective derivatives.

In a preferred embodiment of the sixteenth to the twentieth aspect the protein comprises a modification, whereby the modification is selected from the group comprising phosphorylation and acetylation.

In a twenty-first aspect of the present invention the problem is solved by a method for the regeneration of tissue comprising the following steps:
a) providing a tissue or a part thereof,
b) adding a nucleic acid, the transcription product thereof and/or the translation product thereof; and
c) incubating the tissue and the nucleic acid, the transcription product thereof and/or the translation product thereof,
   whereby the nucleic acid is selected from the group comprising genes for basic DNA-binding proteins, and, optionally,
d) obtaining or recovering the regenerated tissue or a intermediate form thereof.

In an embodiment of the twenty-first aspect the method is an in vitro method.

In an embodiment of the twenty-first aspect the tissue to be regenerated is different or identical to the tissue provided in step a).

In an embodiment of the twenty-first aspect the tissue to be regenerated and/or the tissue provided in step a) is/are independently selected from each other from the group comprising skin tissue, fatty tissue, cartilage tissue, muscle tissue, cells of the blood and of the haemogram and nerve cells.

In an embodiment of the twenty-first aspect the nucleic acid, the transcription product and/or the translation product is/are such as described herein.

In a twenty-second aspect of the present invention the problem is solved by a method for the dedifferentiation and/or reprogramming of cells comprising the following steps:
a) providing one or several cells,
b) adding a nucleic acid, the transcription product thereof and/or the translation product thereof, and
c) incubating the cell and the nucleic acid, the transcription product thereof and/or the translation product thereof,
   whereby the nucleic acid is selected from the group comprising genes for basic DNA-binding proteins.

In an embodiment of the twenty-second aspect the method is an in vitro method.

In an embodiment of the twenty-second aspect the method further comprises the following step:
d) obtaining a dedifferentiated and/or reprogrammed cell.

In an embodiment of the twenty-second aspect the dedifferentiated cell(s) and/or the reprogrammed cell(s) and/or the cell(s) provided according to step a) is/are independently selected from the group comprising cells of the epidermis, cells of the skin, cells of the fatty tissue, cells of the cartilage tissue, cells of the muscle tissue, cells of the blood, cells of the blood-forming tissues and nerve cells.

In an embodiment of the twenty-second aspect the nucleic acid, the transcription product thereof and/or the translation product thereof is as defined herein.

In a twenty-third aspect of the present invention the problem is solved by a pharmaceutical formulation comprising a nucleic acid, a transcription product thereof and/or translation product thereof, as defined herein, and a pharmaceutically suitable carrier.

In a twenty-fourth aspect of the present invention the problem is solved by a carrier material comprising a nucleic acid, the transcription product thereof and/or the translation product thereof, whereby the nucleic acid, the transcription product thereof and/or the translation product thereof is as defined herein.

In an embodiment of the twenty-fourth aspect the carrier material comprises a material selected from the group comprising cellulose, agarose, collagen, silicone, silicon, plastics, gels, hydrogels, matrices based on fibrin, man-made continuous filament yarn, hydrocolloids, lipocolloids, polyurethane, polyurethane resins, plaster, synthetic biomaterials, thermoplastic plastics, zinc glue, polyester foam, polyisobutylene, buffer, stabilizers, bacteriostatics and moisturizers.

In an embodiment of the twenty-fourth aspect the carrier material is serving as an implant or for wound healing.

In a twenty-fifth aspect of the present invention the problem is solved by a wound covering material comprising a basic cover material and a nucleic acid, the transcription product thereof and/or the translation product thereof, whereby the nucleic acid, the transcription product thereof and/or the translation product thereof is/are as disclosed herein.

In an embodiment of the twenty-fifth aspect the cover material is selected from the group comprising hydrocolloidal dressings, calcium alginate dressings, compresses and overlays of activated carbon, overlays of foamed plastic, film dressings, transparent dressings, silicone foam dressings, fleece overlays, hydrocellular dressings, hydroselective wound overlays, absorbing wound pads, spray dressings, gauze of man-made continuous filaments, cotton gauze, paraffin gauze, silver coated wound dressings and hydropolymer/foam dressings.

In a twenty-sixth aspect of the present invention the problem is solved by a cosmetic formulation comprising a nucleic acid, the transcription product thereof and/or the translation product thereof, whereby the nucleic acid, the transcription product thereof and/or the translation product thereof is as disclosed herein, and a carrier phase, whereby the carrier phase is preferably selected from the group comprising creams, fatty ointment, emulsions (oil in water (O/W); water in oil (W/O); water in oil in water (W/O/W)); microemulsions, modified emulsions, nanoparticles/nanoemulsions, liposomes, hydrodispersion gels (hydrogels, alcohol gels, lipogels, tenside gels), gel-creams, lotions, oils/oil baths and sprays.

In a twenty-seventh aspect of the present invention the problem is solved by a method for the screening of a compound for promoting and/or inhibiting a process, whereby the process is selected from the group comprising tissue regeneration, repair of DNA damages, wound healing, cell mobility, angiogenesis in the wound area, epithelialization, tissue aging, inhibition of tissue aging, tissue rejuvenation, vascularization after cardial infarction and healing of tooth and bone implants, comprising the following steps:
a) providing a test system for the process;
b) providing a candidate compound; and
c) testing the candidate compound and determining the reaction caused by the candidate compound in the test system.

In a twenty-eighth aspect of the present invention the problem is solved by a method for the screening of a compound for promoting and/or inhibiting a process, whereby the process is selected from the group comprising tissue regeneration, a repair of DNA damages, wound healing, cell mobility, angiogenesis in the wound area, epithelialization, tissue aging, inhibition of tissue aging, tissue rejuvenation, vascularization healing of tooth and bone implants, comprising the following steps:
a) providing a test system for the process;
b) providing a reference compound;
c) testing the reference compound in the test system and determining the reaction caused by the reference compound in the test system;
d) providing a candidate compound;
e) testing the candidate compound in the test system and determining the reaction caused by the candidate compound in the test system; and
f) comparing the reaction of the reference compound in the test system with the reaction of the candidate compound in the test system.

In a twenty-ninth aspect of the present invention the problem is solved by a method for the screening of a compound for promoting and/or inhibiting a process, whereby the process is selected from the group comprising tissue regeneration, repair of DNA damages, wound healing, cell mobility, angiogenesis in the wound area, epithelialization, tissue aging, inhibition of tissue aging, tissue rejuvenation, vascularization after cardial infarction and healing of tooth and bone implants, comprising the following steps:
a) providing a test system for the process;
b) providing a reference compound, whereby the reference compound comprises a label;
c) testing the reference compound in the test system and determining the reaction caused by the reference compound in the test system;
d) providing the candidate compound; and
e) testing the candidate compound in the test system, whereby the test system contains the reference compound, and determining the reaction of the test system, whereby the amount of released reference compound and/or the amount of the released label of the reference compound is determined.

In a thirtieth aspect of the present invention the problem is solved by a method for the screening of a compound for promoting and/or inhibiting a process, whereby the process is selected from the group comprising tissue regeneration, a repair of DNA damages, wound healing, cell mobility, angiogenesis in the wound area, epithelialization, tissue aging, inhibition of tissue aging, tissue rejuvenation, vascularization after cardial infarction and healing of tooth and bone implants, comprising the following steps:
a) providing a test system for the process;
b) providing a candidate compound, whereby the candidate compound comprises a label;
c) testing the candidate compound in the test system and determining the reaction caused by the candidate compound in the test system;
d) providing a reference compound; and
e) testing the reference compound in the test system, whereby the test system contains the candidate compound, and determining the reaction of the test system, whereby the amount of released candidate compound and/or the amount of released label of the candidate compound is determined.

In an embodiment of the twenty-seventh to the thirtieth aspect the test system is an in vitro test system or an in vivo test system.

In an embodiment of the twenty-seventh to the thirtieth aspect the reaction of the reference compound and/or of the candidate compound is a promotion of the process, and whereby preferably the candidate compound is a compound for promoting the process if the reaction of the candidate compound in the test system is equal to or more pronounced than the reaction of the reference compound.

In an embodiment of the twenty-seventh to the thirtieth aspect the reaction of the reference compound and/or of the candidate compound is an inhibition of the process and whereby preferably the candidate compound is a compound for the inhibition of the process if the reaction of the test system caused by the candidate compound is a reaction which is inferior to the reaction of the test system caused by the reference compound.

In an embodiment of the twenty-seventh to the thirtieth aspect the reference compound is a nucleic acid, the transcription product thereof and/or the translation product thereof, whereby the nucleic acid is selected from the group comprising genes for basic DNA-binding proteins, particularly as disclosed herein.

In a thirty-first aspect of the present invention the problem is solved by the use of a method according to any of the twenty-seventh to the thirtieth aspect for the screening of a compound or the treatment and/or prevention of a disease, whereby the test system provided is a test system for the respective disease.

In an embodiment of the thirty-first aspect the disease is selected from the group comprising those requiring repair of DNA damages, requiring tissue regeneration, requiring wound healing, requiring tooth and bone implants, those going along with tissue aging, wound healing disorders, skin diseases, xeroderma pigmentosum, leather skin, skin cancer, skin after sun burn, skin aging after sun burn, sun burn and cardial infarction.

In a thirty-second aspect of the present invention the problem is solved by the a sun protection agent comprising at least a nucleic acid, the transcription product thereof and/or the translation product thereof, whereby the nucleic acid is selected from the group comprising genes for basic DNA-binding proteins.

In an embodiment of the thirty-second aspect the basic DNA proteins are HMG proteins, particularly those described in any of the preceding claims.

In a thirty-third aspect of the present invention the problem is solved by a compound obtainable by a method according to any of the twenty-seventh aspect or the use according to the thirty-first aspect.

In a thirty-fourth aspect of the present invention the problem is solved by the use of a compound according to the thirty-third aspect for the manufacture of a medicament, preferably for the treatment and/or prevention of a disease as disclosed herein.

In a thirty-fifth aspect of the present invention the problem is solved by a method for the treatment of an organism, characterised in that an effective amount of a DNA-binding protein, of a HMG protein, of a nucleic acid coding therefor or a transcription product thereof and/or a translation product thereof, a functional nucleic acid interacting therewith, a peptide interacting therewith or an antibody interacting therewith and/or a compound according to the thirty-third aspect is administered to the organism.

In an embodiment of the thirty-fifth aspect the organism is suffering from a disease or may suffer from said disease, or to fall ill with the disease, which is preferably a disease as described in any of the preceding claims.

In accordance with the present invention the problem is also solved by the subject matter of the attached, independent claims, whereby particularly preferred embodiments may be taken from the sub-claims.

In accordance with the aspect of the present invention related to the sunscreen it is contemplated that the sunscreen is acting such that it protects against the damaging impact of intense sunlight resulting in sun burn and erythemas, respectively, by reflection or absorption of radiation. The sunscreen according to the invention can be present as an aqueous, alcoholic, oily solutions, emulsions and lotions, creams, fat sticks, gels, aerosols, foam creams and other forms known to the ones skilled in the art. Apart from the DNA binding proteins described herein and the nucleic acid(s) coding therefor, the sunscreens according to the present invention may contain absorbing light blocking agents and/or reflecting agents. Reflecting agents are in particular inorganic compounds such as zinc oxide, ferric oxide, titanium dioxide or calcium carbonate. Light absorbing compounds, also referred to as light filters or UV absorbers, are usually functioning such that the UV radiation is transformed into harmless heat by means of non-radiative inactivation. Preferably one or several of the following compounds and classes of compounds are used: benzophenone derivatives, hydroxy naphthochinones, phenylbenzoxazoles and phenylbenzimidazoles, digalloyltrioleates, amino benzoic acid ester, salicylic acid ester, alicyclic dienones, benzalazine, aromatic urea derivatives, sulfonamides, cumarine derivates or phenylglyoxylic acid derivatives. Further constituents may be mink oil, avocado oil, almond oil, sesame oil, peanut oil, olive oil, saflor oil and/or coconut oil as well as urocanic acid. Further constituents may be, among others, dihydroxyacetone, carotine, walnut shell extracts and further compounds which are particularly suitable to increase skin tanning.

The present invention is based on the surprising finding that basic DNA-binding proteins such as the HMG proteins are suitable to transfer cells into a condition which allows for de-differentiation, differentiation and/or a change in differentiation or a combination of these processes. More precisely, there is a dedifferentiation or reprogramming of a cell under the influence of said proteins which subsequently allows that the cell differentiates optionally into a differentiated condition which corresponds to the condition of the starting cell, or which corresponds to the condition of a different cell, i.e. a cell which is different from the starting cell. In any case the cells are transferred into a reactive condition under the influence of said proteins. This mechanism underlying the various applications and methods of the present invention is in accordance with the observation that said proteins, as so-called masterproteins, control a variety of genes and the functional condition of the cell. Being modulators of transcription factor complexes, they may, in principle, act both in a positive as well as a negative manner on the expression of their target genes. In doing so, their binding to the respective promoters is more structure specific rather than sequence specific and they bend the DNA so that either the binding of transcription factors is mediated or the transcription factors lose their capacity to associate with this portion of the DNA. Due to this characteristic said proteins are also referred to as architectural transcription factors. Additionally, the present inventors have found that said proteins and in particular the HMG proteins are involved in cell and tissue build-up during embryonal and fetal development, whereas after birth they are no longer detectable in most of the differentiated cells. During the early embryogenesis mRNA of some HMG proteins can be detected in nearly all tissues. In the later embryogenesis the expression is limited to some mesenchymal derivatives and some epithelial cell tissues.

As disclosed herein, the described DNA binding proteins, in particular the HMG proteins described herein, the transcription products thereof, the translation products thereof, functional nucleic acids derived therefrom and compounds identified by applying the screening methods disclosed herein, are involved in a variety of biological processes which are in summary referred to herein as processes. These processes as such are known to the ones skilled in the art. More particularly, the involvement of one or several of these processes in diseases or pathological conditions of vertebrates and in particular of mammals and human beings, are known to the ones skilled in the art. It is thus within the present invention that the DNA binding proteins described herein, in particular the HMG proteins described herein, the transcription products thereof, the translation products thereof as well as functional nucleic acids derived therefrom and compounds identified by applying the screening methods disclosed herein, are used for the prevention and/or treatment of diseases or pathological conditions, and for the manufacture of a medicament for the treatment thereof, where one or several of said processes is involved. The diseases disclosed herein, which are also referred to as disorders, are examples therefor. The application of the DNA binding proteins described herein, in particular the HMG proteins described herein, the transcription products thereof, the translation products thereof, functional nucleic acids derived therefrom and compounds identified by applying the screening methods as disclosed herein, is, however, not limited thereto.

It is furthermore within the present invention and obvious for the one skilled in the art that the DNA binding proteins described herein, in particular the HMG proteins described herein, the transcription products thereof, the translation products thereof, functional nucleic acids derived therefrom as well as compounds identified by applying the screening method disclosed herein, may act both in an inhibitory as well as an activating manner and that insofar diseases may be treated in which the processes are desired and are thus promoted or supported by said compounds, as well as those in which said processes are not desired and thus the respective compounds shall be inhibited. In connection therewith, the DNA binding proteins described herein, in particular the HMG proteins described herein, the nucleic acid(s) coding therefor, as well as the transcription factors thereof and the translation factors thereof are preferably used for promoting said processes. It is, however, also within the present invention that the DNA binding proteins disclosed herein, in particular the HMG proteins disclosed herein, the nucleic acid(s) coding therefor as well as the transcription product(s) thereof and translation product(s) thereof may also be used for the inhibition of these processes. If upon administration of the DNA binding proteins disclosed herein, in particular the HMG proteins disclosed herein, the nucleic acid(s) coding therefor as well as the transcription product(s) thereof and the translation product(s) thereof, a deficit thereof is to be compensated or their active concentration is to be increased, this additional administration may also result in inhibition, for example as a competitive inhibition. In contrast thereto, the functional nucleic acids as described herein, in particular antisense molecules, RNAi, aptamers, spiegelmers and aptazymes, which are directed against the DNA binding proteins as disclosed herein, in particular the HMG proteins disclosed herein, the nucleic acid(s) coding therefor, as well as the transcription product(s) thereof and the translation product(s) thereof, are preferably used in the inhibition of the processes which are mediated by them. This is also true for the antibodies, peptides and compounds, which are identified or obtained in the screening methods disclosed herein, which are directed against the DNA proteins described herein, in particular the HMG proteins disclosed herein, the nucleic acid(s) coding therefor, the transcription product(s) thereof and the translation product(s) thereof. In general, the antibodies and peptides which are directed against the DNA binding proteins as disclosed herein, in particular the HMG proteins disclosed herein, the nucleic acid(s) coding therefor, as well as the transcription product(s) thereof and the translation product(s) thereof, may be used in the same way and to the same extent as the functional nucleic acids disclosed herein.

Examples for diseases, where the effect of the DNA binding proteins disclosed herein, in particular the HMG proteins disclosed herein, the nucleic acid(s) coding therefor, as well as transcription product(s) thereof and the translation product(s) thereof is to be promoted, are angiogenesis, cardial infarction by transmyocardial revascularisation, wound healing, angiogenesis following wounding, epithelialization and healing of tooth and bone implants. Diseases where the effect of the DNA binding proteins, in particular the HMG proteins disclosed herein, the nucleic acid(s) coding therefor as well as the transcription product(s) thereof and the translation product(s) thereof, are to be inhibited, are endometriosis, psoriasis, macular degeneration, in particular age dependent macular degeneration, cornea diseases, preferably those of human beings and dogs, going along with angiogenesis, preferably pannus, i.e. chronic superficial keratitis, histiocytosis, preferably acute forms thereof, more preferably those in the field of veterinary medicine.

The use of the DNA binding proteins disclosed herein, in particular of the HMG proteins described herein, the nucleic acid(s) coding therefor, as well as the transcription product(s) thereof and the translation product(s) thereof, the functional nucleic acids, antibodies and peptides directed against them, as well as of the compounds obtained and/or identified by using them, is particularly advantageous if several of the processes are involved in said disease and which are promoted and inhibited, respectively, by them. Examples are psoriasis, macular degeneration and endometriosis as well as pannus of the dog, where both inflammatory processes as well as processes of angiogenesis are involved which are (also) caused by the DNA binding proteins disclosed herein, in particular the HMG proteins disclosed herein, and the nucleic acid(s) coding therefor, whereby in particular the HMGB proteins play an essential role. Insofar, the functional nucleic acids, peptides and antibodies directed against them and against the respective transcription product are suitable means for treatment. This is also true for compounds which inhibit the effect of HMGB proteins and the nucleic acid coding therefor, which may, for example, be identified by a screening method according to the present invention.

The present invention is also based on the surprising finding that the members of the HMGA and the HMGB family can trigger angiogenesis or neovascularization processes. Angiogenesis is promoted to an extent which is comparable to the one of highly specialised factors for angiogenesis such as VEGF. In contrast to the use of highly specialised angiogenic factors such as VEGF, the use of the HMG proteins may result in further effects as will be outlined in the following.

Additionally, the present invention is based on the surprising finding that, from the group of the HMG proteins, in particular HMGB1 and HMGA1 show a strong angiogenic effect and may, accordingly, be used for the treatment of diseases associated with angiogenesis as disclosed herein. This use is based on the surprising finding that the angiogenic effects, as expressed by the length of the sprouts of blood vessels, are surprisingly big. These effects are, in contrast thereto, not to be expected by an HMGB1 induced release of cytokines.

Additionally, the present inventors have surprisingly found that necrotic cells, in particular necrotic tumor cells release HMGB1 and, to a certain extent, also HMGA proteins which stimulate as extracellular ligands through the RAGE receptor endothelial cells for vasculogenesis/angiogenesis. From this mechanism, it may be taken that drugs screened against HMG, in particular HMGB1 and HMGA, more precisely HMGA1, may particularly be used for the treatment of tumor diseases which comprise necrotic cells and necrotic tumor cells, respectively, and that the screening methods described herein will provide such molecules or will be capable of providing such molecules. Furthermore, it may be taken from this mechanism that functional nucleic acids, antibodies or peptides which are directed against the aforementioned proteins and the nucleic acids coding therefor, respectively, may be used as means for the treatment of tumors, in particular tumors which release HMGB1 and/or to a certain extent also HMGA proteins, and/or, as extracellular ligands, stimulate endothelial cells for vasculogenesis and angiogenesis, respectively, by means of the RAGE.

The formation of new blood vessels is important for a variety of processes such as wound healing, tumor growth, neovascularization and the treatment of hypoxia and ischemia in myocardial tissue. Two different mechanisms are distinguished which seem to be basically involved in these processes. One is vasculogenesis, i.e. the new formation of vessels from in situ differentiating endothelial precursor cells, and the other one is angiogenesis, i.e. the new formation of vessels from existing blood vessels. The endothelial cells of mature blood vessels of the adult organism are in a resting, non-proliferative phase. They are only put in a position to participate in the process of angiogenesis and to stimulate the process of angiogenesis, respectively, upon having been stimulated by, for example, infection, trauma, hypoxia or ischemia. Subsequently there is a cascade of different processes following each other which comprise, among others, migration, proliferation and newly connecting endothelial cells. As a result a new, three-dimensional vessel tube is formed. Additionally, myocytes of the smooth masculature of the vessel wall grow in during angiogenesis and neogenesis of vessels which are bigger than capillaries, thereby providing stability to the newly formed vessel.

In particular the growth factors fibroblast growth factor (FGF) and vascular endothelial growth factor (VEGF) are to be taken into considerations as the critical ones from a big variety of growth factors and cytokines involved in angiogenesis. They are known as potent mitogens of endothelial cells. By administering the growth factors and molecules, respectively, which in turn are stimulating distinct growth factors, a better blood flow into the area to be treated is ensured.

A clinically important form of angiogenesis occurs in the process of the so-called transmyocardial laser revascularization (TMLR). In connection therewith, laser pulses create little channels in the heart muscle. Assumingly, the traumatic stimulation of the heart muscle tissue in the vicinity of the thus generated channels results in angiogenesis in its surroundings which ultimately leads to an improved blood flow in the heart muscle, particularly in the area of the ischemic tissue. Transmyocardial revascularization comprises in particular revascularization after cardial infarction, but also revascularization of early stages of heart diseases such ischemia or vascular related heart failure. In connection with the present invention, the terms angiogenesis, vasculogenesis, neovascularization and revascularisation are to be used in a synonymous manner, whereby in any case it is focused on the observed effect, namely that there is blood vessel formation, independent of the underlying molecular or cellular mechanism.

Specifically influencing angiogenesis and neovascularization in the sense of promoting or inhibiting these processes, allows the treatment of diseases related to angiogenesis or neovascularisation.

This applies, for example, to diseases where due to an insufficient blood supply ischemic conditions exist, for example in case of coronary stenosis or artheriosclerosis. In such cases the induction of new blood vessels by means of angiogenesis or neovascularization could provide for alternative vessels for blood supply and thus contribute to an alleviation of the disease. Further examples for diseases which are caused by an insufficient blood supply, comprise growths, badly healing or chronic wounds, complication of pregnancy such as gestosis and infertility.

In other cases, the diseases may be based on a excessive formation of blood vessels. An example therefor is proliferative retinopathia diabetica in connection with which the new formation of vessels on and in front of the retina may result in loss of sight. A further example is the formation of tumors. In the meantime it is well known that a successful tumor growth requires a sufficient supply of the tumor cells with nutrients which in turn requires a sufficient supply of the tumor site with blood vessels. Tumors which are not capable of inducing a sufficient supply of blood vessels are therefor limited in their growth. In contrast thereto, the growth of tumors can be actively limited by stopping the supply with blood vessels. Particularly in connection with skin quite a number of cancer-like diseases have been linked to excessive angiogenesis, such as basalioma, squamous carcinoma, melanoma, Karposi's sarcoma as well as actinic keratoma as precursor to cancer. In principle, any kind of cancer and tumor can be regarded as going along with excessive angiogenesis. Further examples for diseases caused by excessive angiogenesis are psoriasis as well as arthritis.

In connection with the present invention the term disorder shall not only comprise disorders or diseases in the classical sense such as retinopathy, psoriasis or tumors, but generally refers to conditions the abolishment of which is desirable in the sense of increasing a patient's comfort. This comprises in particular artificially generated wounds such as those happening in connection with surgery, for example surgical wounds or wounds caused by the implantation of protheses or implants.

Angiogenesis also occurs in a controlled manner which is coordinated by a variety of cytokines and growth factors, in connection with wound healing which is a dynamic process with complex interactions between cells, the extracellular matrix and plasma proteins. It is the task of angiogenesis to provide supply paths by which nutrients and other substance are transported to the cells in the wound area, whereby these supply paths may be maintained after completion of the healing process and may optionally be reduced in numbers to maintain the supply of the healed tissue. Independent from the kind of the wound and the extent of tissue loss, wound healing may be grouped into timely overlapping phases of inflammatory and exudative phase, respectively, the proliferative phase as well as differentiation and re-organisation phase. This grouping into phases is based on the basic morphological changes in the course of the repair processes without actually reflecting the complexity of the processes.

The process of wound healing can be divided into primary and secondary wound healing, whereby, in order to take into account the therapeutic problems resulting from the extent and the kind of tissue damage, delayed primary healing as well as chronic wound course are furthermore distinguished. Primary wound healing is, for example, given in case of smooth, closely facing wound surfaces of a cut without essential loss of tissue and without any incorporation of xenoliths in a tissue which is well supplied with blood vessels. Primary wound healing is typically realised in connection with surgical wounds or occasional wounds by sharp-edged objects. If infection has to be taken into consideration due to the way the wound was generated, a delayed primary healing occurs. If there is an infection, the wound is categorised as secondary healing. Secondary healing is present in case of bigger defects in which granulation tissue has to be build-up or where an infection does not allow for the immediate approximation of the wound edges. If the healing is not completed within eight weeks, it is referred to as a chronic healing course. A chronic wound may arise in any wound healing phase and usually results from progressive tissue destruction due to tissue diseases of different origin, local pressure damages, radiation damages or tumors.

A further differentiation of wound healing can be based on discriminating between acute wounds and chronic wounds. Acute wounds range from acute traumatic wounds to complex traumatic defects, thermal and chemical injuries/burns and incisions/surgical wounds.

In acute traumatic wounds, a primary closing of the wound occurs by means of suture, staples or wound approximation stripes in case that the wound edges may be adapted without tension, optionally after wound excision. In case of wounds which show a potential risk of infection, the wound is first kept open using sterile humid dressings until an infection can be excluded. In secondary healing and more complex wounds the wound closing is more complex.

In case of thermal and chemical wounds, such as those arising from the impact of heat and cold or tissue damaging radiation, acids or bases, treatment occurs in accordance with the damaging pattern. For example, heavily burned patients first undergo a necrectomy with subsequent surgical replacement by a skin transplant. In case the wound cannot be transplanted or in case there are not sufficient donor areas available due to extensive burn, so-called allo- or xenotransplants are used. If there are sufficient donor areas, permanent autologous skin transplantation can be used. A particular form is the autologous keratinocyte transplantation.

Chronic wounds are secondary healing wounds which, despite causal and appropriate local therapy, do not heal within eight weeks. Although chronical wounds can develop at any time from an acute wound, most of the chronic wounds are the final stadium of a progressing tissue destruction which is caused by venous, arterial or metabolism caused vascular disorders, pressure damages, radiation damages or tumors. The various types of chronic wounds are caused by different pathologies, whereby the wounds are regarded as similar in terms of biochemistry. The local factors which influence wound healing are, among others, xenoliths, ischemia, repeated traumata and infections. Furthermore, systemic factors such as increased age, undernutrition and malnutrition, diabetes as well as renal diseases may have an impact on wound healing. The economically most relevant chronic wound healing disorders are, among others, ulcus cruris venosum, ulcus cruris arteriosum, diabetic ulcer, decubital ulcer and the chronic post-traumatic wound.

An important cause for chronic wounds is an imbalance between repair processes which result in the formation of new tissue, and destructive processes which result in the removal of damaged tissues. For example, an increased protease activity such as an overexpression of matrix metalloproteases, may result in a controlled degradation of the extracellular matrix. The imbalance between synthesis and degradation of the extracellular matrix and the resulting shift of the wound balance in direction of destructive processes can be removed by, among others, the proliferation promoting effect of the basic DNA binding proteins and in particular the HMG proteins. Compared to the use of single exogenous growth factors which typically induce a specific signal cascade, said proteins are advantageous insofar as they act as architectural transcription factors on a variety of different proliferation promoting signal transduction pathways and thus exhibit a broader spectrum of activity. Said proteins may induce the synthesis of a variety of functional proteins in different cells such as keratinocytes, fibroblasts and endothelial cells. Furthermore, said proteins interact with the signal cascade at a later stage compared to the initially acting exogenous growth factors which are in many cases rapidly degraded, assumingly by the high protease content of chronic wound exudate.

The treatment of the various wounds can basically be grouped into active and passive wound therapy. As a particular form skin replacement methods may also be applied. In passive wound therapy inactive, textile dressings are used which provide protection against infection as a mere cover material. In contract to inactive dressing, interactive wound covers typically provide for a humid wound environment and thus promote the healing process. In connection therewith, hydrocolloids, hydrogels, hydropolymers and foam dressings as well as calcium alginate are, among others, used. The disadvantage of this passive wound therapy is that the dressing material does not promote the active healing of problematic wounds, in particular chronic wounds, which are frequently thought as being therapy resistant.

The high molecular compounds which are mentioned in the prior art and used for wound healing such as growth factors, cytokines, blood clotting factors and the like, selectively intervene with the processes of wound healing and skin aging. Using the basic DNA binding proteins such as the HMG proteins, however, this can result, due to the central mode of action of said proteins at the very beginning of the differentiation condition, in a comprehensive regeneration of the tissue and the individual cells, respectively. The observed surprising effect of said proteins in terms of wound healing in the broader sense, as defined herein, is based on the following processes and mechanisms, whereby it may be contemplated that said proteins interact both in the proliferative as well as differentiation and re-organisation phase of the wound healing processes, including building up of granulation tissue, stimulation of angiogenesis as well as proliferation and migration of epithelial cells, and may be used for these processes and in methods based on these processes. Accordingly, said proteins and the nucleic acids coding therefor, may be used in diseases which are associated with these processes and phases, respectively, which go along with them, which make use thereof and/or which are based thereon in a causal or symptomatic manner.

A sufficient blood flow of the wound area is furthermore of critical importance for the healing process. If it is strongly reduced, a sufficient wound metabolism cannot be provided which may result in a chronic healing course. The basic DNA binding proteins and in particular the HMG proteins also promote angiogenesis in the wound area by inducing the proliferation of endothelial cells. Finally, senescence of cells plays a particular role in wound healing disorders. An increasing age of dermal fibroblasts correlate with a reduced proliferation potency. Fibroblasts in chronic wounds show a worsened reaction on growth factors which is most likely based on an increasing number of senescent cells. Said proteins have the capability to put these cells again into an active condition and to reactivate the proliferation by the reprogramming or rejuvenating capabilities.

Finally, a strongly reduced epithelialization represents a further disturbance of the healing of chronic wounds, whereby the healing cannot be completed. One factor is the limited migration of epithelial cells at the immediate edge of the ulcus. The present inventors have shown that HMG proteins may increase the mobility of cells so that there is also a positive effect on the migration of epithelial cells by the HMG proteins.

As used herein, the basic DNA binding proteins exhibit the functions described herein.

HMG proteins were named due to their high electrophoretic mobility in polyacrylamide gels. They belong to the chromosomal non-histone proteins and are primarily not defined by their protein function but by their chemical and physical characteristics. All members of these proteins may be extracted from chromatin by 0.35 M NaCl, are soluble in 2 to 5% perchloric acid, have a high content of charged amino acids and a molecular weight of less than 30,000 Da. The HMG proteins are divided into three subgroups taking into consideration their sequence homology and sequence motifs, namely the HMGB (formerly HMGB-1/2) family, the HMGN (formerly HMG-14/17) family and the HMGA (formerly HMG-I/Y/C) family.

The members of the HMGN family are expressed in all higher eukaryotes. Their molecular weight is between 10,000 and 20,000 Da. They are the only non-histone proteins binding with a higher affinity with their positively charged nucleosome binding domain (NBD) to the nucleosome core than histone-free DNA. This binding domain comprises amino acids 12 to 41 of the HMGN1 protein and amino acids 17 to 47 of the HMGN2 protein and have also been found in the sequence of other proteins; accordingly, for example, NBP45 contains a NBD motif in its primary sequence.

The HMGA family consists of three members, namely HMGA1a and HMGA1b, which are the two splice variants of one gene, and the related protein HMGA2 coded by a different gene. The average molecular weight of the proteins of the HMGA family is between 10,000 and 12,000 Da. The members of this protein family are normally found only in undifferentiated embryonal cells, in neoplastic cells as well as during the exponential growth phase of differentiated cells. In contrast, they are hardly detectable or only at very low concentrations in differentiated cells of normal tissue.

Proteins of this family posses each three separate DNA binding domains as well as an acidic protein binding domain. The HMGA proteins bind to the small grove of AT-rich DNA. The binding of HMGA at genes the promoter/enhancer sequence of which is localised in the vicinity of such HMGA binding sites, may result in influencing transcription. For example, acetylation of HMGA1 plays a critical role in the regulation of the enhanceosome complex for the transcripton of the interferon-beta (IFN-beta) gene. Further post-translational modifications of the HMGA proteins are phosphorylation which is dependent on the cell cycle, and ADP-ribosylation, respectively.

The members of the HMG family belong to the most frequent HMG proteins. The average molecular weight is 25,000 Da at maximum.

HMGB proteins consist of three domains, whereby the two conservative domains which have a high sequence homology, represent the inspecific DNA binding region of the proteins. This functional motif is referred to as HMG box. The HMGB proteins contain two of these HMG boxes, namely Box A and Box B. The C terminal portion of the HMGB1 protein forms the protein binding domain of the HMGB proteins. Apart from the HMGB proteins there is a great variety of other proteins, where the HMG boxes can be detected. This group comprises SRY, SOX proteins, LEF1 and UBF1 (A. D. Baxevanis and D. Landsmann: The HMG-1 box protein family: classification and functional relationships. NAR 23, 2002, 1604-1613). The HMG proteins are structural components of the chromatin and are involved in transcriptional regulation.

In principle, all HMG proteins both those already known and those which still have to be found in the future, may be applied in connection with the present invention, in particular after performing the experiments described herein, in order to determine for the individual case whether the particular HMG protein shows the respective characteristics in accordance with the present invention and thus the behaviour in connection with the respective application.

For the time being, there are about 15 HMG proteins known. The proteins of the HMGB family, of the HMGA family and/or proteins of the HMGB family, the latter ones particularly in combination with proteins of the HMGA family, are particularly preferred for the applications and uses as described herein. It is within the present invention that the proteins of said families cannot only be used at the level of the translation products, but also at the level of the transcription products or of the genes and coding sequences, respectively.

It is within the present invention that aberrant transcripts of the HMG proteins are used. Such truncated HMG proteins are described in the literature, among others, also in international patent applications WO 96/25493 or WO 97/23611, the disclosure of which is incorporated herein by reference.

Preferably the truncated HMG proteins as they may be used in connection with the present invention, exhibit at least exon 1, preferably at least exons 1 to 3 of the HMGA2 gene, as well as further amino acids, which are coded by sequences which originate from the regions of different chromosomal translocation partners of chromosome 12. Both these truncated forms as well as further derivatives of the HMG proteins such as, for example, HMGA2-LPP; HMGA2-RAD51L1 (described, for example, in Tkachenko, A et al., Cancer Res 997; 57 (11): 2276-80; Schoenmakers E F et al.; Cancer Res 1999 59 (1): 19-23) HMGA1-LAMA4 (for example described in Schoenmakers E F et al. supra; Tkachenko, A et al., supra) und SP100-HMGB1, in particular HMGA1a, HMGA1b as well as HMGA2 may be present as derivatives. Such derivatives may, for example, be produced by means of post-translational modifications such as acetylation or phosphorylation, however, also by conjugation to other molecules, whereby it is within the present invention that when several proteins are used simultaneously, they may be independently from each other provided with the same or different modifications or whereby not each and any of them are simultaneously modified. Such other molecules may, for example, be selected from the group comprising sugars, lipids, peptides and small organic molecules having a molecular weight of less than 1,000.

Preferred HMG proteins which may be used within the various aspects of the present invention, are each and any of those described in the following table 1 which are referred to by SEQ. ID. NOs. 1 to 30. Table 1 provides an overview presenting the SEQ. ID. NOs., the amino acid length, the databank accession number, to the extent known, as well as the design of the exon structure and amino acids which are added, if any.

| SEQ | Name of the | Length of the | | | accession |
|---|---|---|---|---|---|
| ID NO. | protein | protein | | exon structure | number |
| 1 | HMGA1a protein | 107 | amino acids | | X14957 |
| 2 | HMGA1b protein | 96 | amino acids | | X14958 |
| 3 | HMGA2 protein | 109 | amino acids | | P52926 |
| 4 | trunkiertes HMGA2 | 83 | amino acids | Exon 1-3 | |
| 5 | truncated HMGA2: | 90 | amino acids | Exon 1-3 + 7 | U29113 |
| | IC113 ORF | | | amino acids | |
| 6 | truncated HMGA2: | 96 | amino acids | Exon 1-3 + 13 | U29117 |
| | IC117 ORF | | | amino acids | |
| 7 | HMGB1 protein | 215 | amino acids | | S02826 |
| 8 | truncated HMGA2 | 147 | amino acids | Exon 1-3 + 64 | U29119 |
| | | | | amino acids | |
| 9 | truncated HMGA2 | 106 | amino acids | Exon 1-3 + 23 | U29112 |
| | | | | amino acids | |
| 10 | truncated HMGA2 | 92 | amino acids | Exon 1-3 + 9 | H98218 |
| | | | | amino acids | |
| 11 | truncated HMGA2 | 96 | amino acids | Exon 1-4 + 2 | U29120 |
| | | | | amino acids | |
| 12 | truncated HMGA2 | 118 | amino acids | Exon 1-4 + 24 | U29115 |
| | | | | amino acids | |
| 13 | truncated HMGA2 | 95 | amino acids | Exon 1-4 + 1 | U29114 |
| | | | | amino acids | |
| 14 | HMGA1a AT-Hook 1 | 11 | amino acids | coded by exon 5, | X14957 |
| | | | | position in protein | |
| | | | | AS 21-31 | |
| 15 | HMGA1a AT-Hook 2 | 11 | amino acids | coded by exon 6, | X14957 |
| | | | | position in protein | |
| | | | | AS 53-63 | |
| 16 | HMGA1a AT-Hook 3 | 12 | amino acids | coded by exon 7, | X14957 |
| | | | | position in protein | |
| | | | | AS 78-89 | |
| 17 | HMGA1b AT-Hook 1 | 11 | amino acids | coded by exon 5, | X14958 |
| | | | | position in protein | |
| | | | | AS 21-31 | |
| 18 | HMGA1b AT-Hook 2 | 11 | amino acids | coded by exon 6, | X14958 |
| | | | | position in protein | |
| | | | | AS 42-52 | |
| 19 | HMGA1b AT-Hook 3 | 12 | amino acids | coded by exon 7, | X14958 |
| | | | | position in protein | |
| | | | | AS 67-78 | |
| 20 | HMGA2 AT-Hook 1 | 11 | amino acids | coded by exon 1, | P52926 |
| | | | | position in protein | |
| | | | | AS 24-34 | |
| 21 | HMGA2 AT-Hook 2 | 11 | amino acids | coded by exon 2, | P52926 |
| | | | | position in protein | |
| | | | | AS 44-54 | |
| 22 | HMGA2 AT-Hook 3 | 21 | amino acids | coded by exon 3, | P52926 |
| | | | | position in protein | |
| | | | | AS 71-91 | |
| 23 | HMGB1 HMG-BOX A | 78 | amino acids | coded by exon 2 + | S02826 |
| | (LARGE) | | | 3, position in | |
| | | | | protein AS 6-83 | |
| 24 | HMGB1 HMG-BOX A | 71 | AS (P09429) | coded by exon 2 + | P09429 |
| | (SMALL) | | | 3, position in | |
| | | | | protein AS 9-79 | |
| 25 | HMGB1 HMG-BOX A | 73 | amino acids | coded by exon 2 + | NP_002119 |
| | (MEDIUM) | | | 3, position in | |
| | | | | protein AS 6-78 | |
| 26 | HMGB1 HMG-BOX B | 75 | amino acids | coded by exon 3-5, | S02826 |
| | (LARGE) | | | position in protein | |
| | | | | AS 92-166 | |
| 27 | HMGB1 HMG-BOX B | 69 | amino acids | exon 3-5, | P09429 |
| | (MEDIUM) | | | position in protein | |
| | | | | AS 95-163 | |
| 28 | HMGB1 HMG-BOX B | 49 | amino acids | coded by exon 3 + | NP_002119 |
| | (SMALL) | | | 4, position in | |
| | | | | protein AS 95-143 | |
| 29 | SP100-HMGB1 | 181 | amino acids | alternative exon | AF076675 |
| | | | | (HMGB1L3) | |
| 30 | HMGA2-LPP | 225 | amino acids | exon 1-3 + 142 | |
| | | | | amino acids | |

The nucleic acids used in connection with the various aspects of the present invention are nucleic acids and their transcription products, respectively, which code for the HMG proteins as described herein, or their derivatives. It is within the present invention that any nucleic acid is comprised which codes for the aforementioned HMG proteins. Respective nucleic acids may be comprised through the degeneracy of the genetic code. Particularly preferred nucleic acids are those to which it is referred by using the SEQ. ID. NOs. Table 2 provides an overview of various DNA binding proteins and nucleic acids coding therefore, which are particularly preferred. It is also within the present invention that said nucleic acids are those that hybridise with the afore described nucleic acids and their transcription factors, respectively, and/or their complementary strands, in particular under stringent hybridisation conditions. Such stringent hybridisation conditions are, for example, those realised in 0.1×SSC/0.1 SDS at 68° C. (Perfect Hyb™ Plus (hybridisation buffer of the company Sigma)) or in 5×SSC/50% formamide/0.02% SDS/2% blocking reagent/0.1% N-lauroyl sarcosine at 42° C. over night.

Furthermore, those nucleic acids are comprised which have an identity of at least 65, preferably 70, 75, 80, 85, 90, 95, 98 or 99% to said nucleic acids. A transcription product as used herein is particularly also a hnRNA or an mRNA and cDNA, respectively, for the HMG protein coding nucleic acids, as described herein.

It is also within the present invention that inhibitory sequences which are derived from the nucleic acid sequences of genes of HMG proteins, such as antisense nucleic acids, ribozymes or RNAi are used. Antisense nucleic acids, which are usually used as antisense oligonucleotides, show base complementarity to a target RNA, preferably the mRNA of a gene to be expressed, and hybridise because of this with said target RNA, whereby the enzyme RNase H is activated which results in degradation of the nucleic acids. Ribozymes are catalytically active nucleic acids which preferably consist of RNA and comprise two subportions. The first subportion is responsible for a catalytic activity, whereas the second subportion is responsible for specific interaction with the target nucleic acid. If there is an interaction between the target nucleic acid and the second portion of the ribozyme which is typically based on the hybridisation of stretches consisting of essentially complementary bases, then the catalytic part of the ribozyme can hydrolyse the target nucleic acid either intramolecularly or intermolecularly, provided that the catalytic effect of the ribozyme is a phosphodiesterase activity. As a result, the coding nucleic acid is degraded and, finally, the expression of the target molecule is reduced both at the level of transcription as well as at the level of translation. RNAi is a double-stranded RNA which mediates RNA interference and typically has a length of about 21 to 23 nucleotides. One of both strands of the RNA corresponds to the sequence of a gene to be degraded. The introduction of an RNAi one strand of which is complementary to preferably the mRNA of a gene, may result in reduction of the expression of the gene. The generation and use of RNAi molecules as a medicament and diagnostic means, respectively, is, for example, described in international patent applications WO 00/44895 and WO 01/75164.

TABLE 2
Preferred nucleic acids coding for the DNA-binding proteins

| SEQ | Name of the | Number of | | Accession |
|---|---|---|---|---|
| ID NO. | nucleic acid | base pairs | Exon structure | number |
| 31 | HMGA1a mRNA | | | M23614 |
| 32 | HMGA1a coding Sequence | 324 | | M23614 |
| 33 | HMGA1b mRNA | | | M23616 |
| 34 | HMGA1b coding Sequence | 291 | | M23616 |
| 35 | HMGA2 mRNA | | | NM_003483 |
| 36 | HMGA2 coding Sequence | 330 | | NM_003483 |
| 37 | truncated HMGA2 | 252 | Exons 1-3 + 3 bp | |
| | | | STOP-Codon | |
| 38 | truncated HMGA2: | 273 | Exons 1-3 + 21 | U29113 |
| | IC113 ORF | | base pairs + 3 bp | |
| | | | STOP-Codon | |
| 39 | truncated HMGA2: | 291 | Exons 1-3 + 39 | U29117 |
| | IC117 ORF | | base pairs + 3 bp | |
| | | | STOP-Codon | |
| 40 | HMGB1 mRNA | | | NM_002128 |
| 41 | HMGB1 coding Sequence | 648 | | NM_002128 |
| 42 | truncated HMGA2 | 444 | Exon 1-3 + | U29119 |
| | | | 192 bp + 3 bp | |
| | | | STOP-Codon | |
| 43 | truncated HMGA2 | 321 | Exon 1-3 + | U29112 |
| | | | 69 bp + 3 bp | |
| | | | STOP-Codon | |
| 44 | truncated HMGA2 | 279 | Exon 1-3 + | H98218 |
| | | | 27 bp + 3 bp | |
| | | | STOP-Codon | |
| 45 | truncated HMGA2 | 291 | Exon 1-4 + | U29120 |
| | | | 6 bp + 3 bp | |
| | | | STOP-Codon | |
| 46 | truncated HMGA2 | 357 | Exon 1-4 + | U29115 |
| | | | 72 bp + 3 bp | |
| | | | STOP-Codon | |
| 47 | truncated HMGA2 | 288 | Exon 1-4 + | U29114 |
| | | | 3 bp + 3 bp | |
| | | | STOP-Codon | |
| 48 | HMGA1a AT-Hook 1 | 33 | coded by exon 5, | M23614 |
| | | | position 61-93 | |
| | | | in CDS | |
| 49 | HMGA1a AT-Hook 2 | 33 | coded by exon 6, | M23614 |
| | | | position 157-189 | |
| | | | in CDS | |
| 50 | HMGA1a AT-Hook 3 | 36 | coded by exon 7, | M23614 |
| | | | position 232-267 | |
| | | | in CDS | |
| 51 | HMGA1b AT-Hook 1 | 33 | coded by exon 5, | M23616 |
| | | | position 61-93 | |
| | | | in CDS | |
| 52 | HMGA1b AT-Hook 2 | 33 | coded by exon 6, | M23616 |
| | | | position 124-156 | |
| | | | in CDS | |
| 53 | HMGA1b AT-Hook 3 | 36 | coded by exon 7, | M23616 |
| | | | position 199-234 | |
| | | | in CDS | |
| 54 | HMGA2 AT-Hook 1 | 33 | coded by exon 1, | NM_003483 |
| | | | position 70-102 | |
| | | | in CDS | |
| 55 | HMGA2 AT-Hook 2 | 33 | coded by exon 2, | NM_003483 |
| | | | position 130-162 | |
| | | | in CDS | |
| 56 | HMGA2 AT-Hook 3 | 63 | coded by exon 3, | NM_003483 |
| | | | position 211-273 | |
| | | | in CDS | |
| 57 | HMGB1 HMG-BOX A | 234 | coded by exon 2 + | U51677 |
| | (LARGE) | | 3, position 16-249 | |
| | | | in CDS | |
| 58 | HMGB1 HMG-BOX A | 213 | coded by exon 2 + | P09429 |
| | (SMALL) | | 3, position 25-237 | |
| | | | in CDS | |
| 59 | HMGB1 HMG-BOX A | 219 | coded by exon 2 + | NM_002128 |
| | (MEDIUM) | | 3, position 16-234 | |
| | | | in CDS | |
| 60 | HMGB1 HMG-BOX B | 225 | coded by exon 3-5, | U51677 |
| | (LARGE) | | position 274-498 | |
| | | | in CDS | |
| 61 | HMGB1 HMG-BOX B | 207 | coded by exon 3-5, | P09429 |
| | (MEDIUM) | | position 283-489 | |
| | | | in CDS | |
| 62 | HMGB1 HMG-BOX B | 147 | coded by exon 3 + | NM_002128 |
| | (SMALL) | | 4, position 283-429 | |
| | | | in CDS | |
| 63 | SP100-HMGB1 mRNA | 546 | | AF076675 |
| 64 | HMGA2-LPP CDS | 678 | Exon 1-3 + | |
| | | | 426 bp + 3 bp | |
| | | | STOP-Codon | |

It is within the present invention that preferably human HMG proteins and the nucleic acids coding therefor, are used. Due to the sequence homology and the high degree of conservation of the HMG proteins, it is however within the present invention that said proteins and the nucleic acids coding therefor are those which originate from organisms or species different from man. These are in particular those from other mammals, preferably those from dog, cat, mouse, rat, horse, cattle and pig. Further sources for the HMG proteins used in accordance with the present invention, are those of fish, amphibians, reptiles and birds. Particularly preferred are fish, in particular salt water fish. Further preferred sources are cartilage fish and bone fish.

The applications and uses in accordance with the present invention of the DNA binding proteins as described herein, in particular of the HMG proteins described herein and the nucleic acids coding therefor, including the transcription products thereof and/or the translation product thereof, extend to a variety of processes. Preferably the processes are those which are selected from the group comprising angiogenesis, neovascularization, transmyocardial revascularization, wound healing, tissue regeneration, cell mobility, angiogenesis, in particular angiogenesis in the wound area, epithelialization, tissue aging, vascularization, in particular vascularization in connection with cardiac infarction, healing of tooth and bone implants, dedifferentiation of cells and tissue, respectively, differentiation of cells and tissues, respectively, and combinations of dedifferentiation and differentiation processes. The DNA binding proteins as described herein, in particular the HMG proteins as described herein and the nucleic acids coding therefor, have the capability to initiate, support, maintain and/or to continue one or several of the aforementioned processes, whereby it is within the present invention that the aforementioned processes are inhibited by the antisense molecules, ribozymes or RNAi molecules (referred to herein generally as functional nucleic acids) generated on the basis of the nucleic acid sequences of the HMG genes, or by compounds identified on the basis of the screening processes disclosed herein. The various processes may be simultaneously, but also in a timely arranged manner and optionally in an overlapping manner performed using said proteins and the nucleic acids coding therefor. Without wishing to be bound thereto, it seems that the processes disclosed herein and in particular the aforementioned processes share as a common feature that the basis therefor reside in an activation of the cells involved in the various processes. Basically, all these processes can be addressed by said proteins and the nucleic acids coding therefor, in the sense that they can be initiated, triggered, supported, maintained and/or amplified. The application and use in accordance with the present invention of the DNA binding proteins as described herein, in particular of the HMG proteins as described herein and the nucleic acids coding for said proteins as well as molecules derived therefrom, in particular antisense molecules, ribozymes, RNAi molecules and inhibitors identified by applying the screening methods as disclosed herein, also extend to those diseases which are associated with one or several of the processes described herein in a causal or symptomatic manner, and can be used for the manufacture of respective medicaments, pharmaceutical formulations or cosmetic formulations, respectively. Said use and application in accordance with the present invention may be an in vivo and/or in vitro and/or in situ use. It is within the present invention that this application and use, respectively, are disclosed independent of the underlying mechanism. Furthermore, the use of the DNA binding proteins as described herein, in particular the HMG proteins as described herein and the nucleic acids coding therefor, including the transcription products thereof and/or the translation products thereof, and including the functional nucleic acids disclosed herein and the compounds identified by the screening methods disclosed herein, in combination with known angiogenic factors such as VEGF is within the present invention and the various aspects thereof, including in particular the use aspects of the present invention.

With regard to the complex interaction of the afore-described factors like blood flow, age of fibroblasts present in the wound area and the epithelialization of the wound, in one aspect the invention contemplates the use of a nucleic acid, transcription products or translation products of proteins of the HMGA family together with nucleic acids, transcription products or translation products of the proteins of the HMGB family. Without wishing to be bound by any theory, the present inventors assume that the nucleic acids, transcription products or translation products of proteins of the HMGA family which is a protein family essentially expressed in angiogenesis, effect dedifferentiation or rejuvenation of cells and tissue which ultimately also increases the proliferation potency of fibroblasts and the migration of the epithelial cells. Nucleic acids, transcription products or translation products of the proteins of the HMGB family, in particular of HMGB1, trigger essentially a signal cascade which results in angiogenesis and neovascularization. These different effects of members of the HMGA family and of the HMGB family result in a synergistic effect with regard to angiogenesis or neovascularization and wound healing.

Preferred disorders and diseases, respectively, for the treatment and/or prevention of which the DNA-binding proteins as described herein may be used, particularly the HMG-protein described herein and the nucleic acids coding therefore, as defined herein, as well as the functional nucleic acids disclosed herein and the compounds identified using the screening methods disclosed herein, and for which for the medicaments or pharmaceutical formulations disclosed herein may be used, are particularly the following ones: diabetic retinopathy, proliferative retinopathia diabetica, diabetic nephropathy, macular degeneration, arthritis, psoriasis, endometriosis, rosacea, small varicose veins, eruptive haemangioma, cavemoma, lip angioma, hemangiosarcoma, haemorrhoids, artherosclerosis, angina pectoris, ischemia, infarction, basalioma, squamous carcinoma, melanoma, Karposi's arcoma, tumors, gestosis, infertility, cornea diseases, in particular cornea diseases of man and dog, in particular those diseases going along with angiogenesis, pannus (chronic superficial keratitis), histiocytosis, preferably acute forms thereof, more preferably in the veterinary medicine, primary and secondary wound healing, disturbed primary and secondary wound healing, chronic would healing, acute wounds and chronic wounds, traumatic wounds, complex traumatic defects, thermal injuries, thermal burns, chemical injuries, chemical burns, incisions, surgical wounds, ulcus cruris venosum, ulcus cruris arteriosum, diabetic ulcer, decubital ulcer, chronic post-traumatic wounds, chronic photo damage, sun burn (dermatitis solaris), skin cancer, skin cancer after sun burn, skin aging after sun burn, corium, xeroderma pigmentosa, burns by thermal radiation, particularly by UV-radiation, as well as any of these diseases in particular patient groups, whereby the patient groups are particularly those, comprising elderly people, people with undernutrition, people with malnutrition, people with diabetes, people suffering from lack of skin pigmentation, people suffering from hypopigmentation, people suffering from excessive pigmentation, people who have undergone radiation therapy or are undergoing radiation therapy, and/or people suffering from renal diseases.

Without wishing to be bound thereto in the following, it seems that the aforementioned repair process of DNA damages is a particular process within the group of the aforementioned processes insofar that it seems that cell damages can be cured which would otherwise result in an organic defect. Under influence of the basic DNA binding proteins described herein and the nucleic acid(s) coding therefore, a naturally occurring repair of DNA damages can thus usually be supported or initiated. Such DNA damages arise in particular after UV exposition, however also after exposition to high-energy radiation in general. This comprises, among others, radioactive radiation as, for example, occurring during radiation therapy for tumor diseases. Further fields of application are, for example, radiation damages due to accidents in the handling of radioactive substances. The mechanism of the basic DNA proteins and in particular of the HMGA proteins underlying this process seems to be particularly advantageous insofar as it does not make use of individual compounds of the repair system, but the cell is enabled by direct access to the chromatin to active its own repair systems and thus provides the basis for a comprehensive repair program for the removal of DNA damages. The respective application may occur both in vitro as well as in vivo with the intention to obtain and restore cell material which no longer comprises the DNA damages or, at least, comprises the same to a reduced extent only.

In connection with angiogenesis or neovascularization it is within the present invention that they are related to tissues or organs which may be provided by explanation. For example, tissues or organs intended for implantation may be stimulated for angiogenesis or neovascularization by using the methods according to the present invention. After implantation the thus treated organs and tissues have a higher chance to grow into the recipient organism and to limit or cure cell or tissue damages which might have occurred during the explanation phase, due to the already induced angiogenesis or neovascularization.

It is also within the present invention that the in vitro cultivated tissues or organs are stimulated for angiogenesis or neovascularization.

In connection with cell and tissue regeneration, it is within the present invention that those cells or tissues are regenerated which are essentially also used as starting material. For example, if during wound healing a defective skin tissue is treated, the regeneration of skin tissue will be preferred which, without wishing to be bound thereto in the following, is essentially caused by the afore-described effects of the HMG proteins. The tissue regeneration or reprogramming of the cells involved therein, can be promoted by the administration of exogenous stimulating agents. Such stimulating agents may, for example, be provided by the tissue into which the reprogrammed cell(s) according to the present invention are implanted. However, it is also within the present invention that such stimulating agents, in particular chemical agents, which are effective as such or which may be effective as such, are contacted individually or in combination, optionally in the form of a respective precursor molecule, with the reprogrammed cell in order to influence the direction into which the cell is to differentiate or to change.

The angiogenic and neovascularizing effect, respectively, the proliferation promoting effect of the DNA binding proteins as described herein, in particularly of the HMG proteins as described herein, as already mentioned, as well as an acceleration of the wound healing process play an important role in would healing. The use of said proteins is particularly advantageous with regard to the kind of scar formation. Compared to post-natal healing, foetal wound healing is a very quick wound healing without scar formation. With regard to the fact that when using said proteins, particularly a combination of proteins from the HMGB and the HMGA family, there is an active wound healing which reflects the process of foetal wound healing, a quick and scar free closing of the wound can be obtained when using said proteins in accordance with the present invention. An important aspect for each and any of the processes in connection with which said proteins are used in accordance with the present invention, is the fact that the risk for skin irritations as well as allergic reactions can be estimated as being extremely low as HMG proteins are natural, endogenous substances. Additionally, said proteins of man and animals are nearly identical due to a high level of conservation so that the results from in vivo experiments from a test animal can be transferred to man with very high reliability. It is also within the wound healing that when the DNA binding proteins described herein, particularly the HMG proteins described herein and the nucleic acids coding therefore, are used in accordance with the present invention, this will allow for the transplantation of a skin substitute, whereby the skin substitute is one which, starting from autologous skin cells and an optional propagation thereof, is generated using said proteins as in vitro stimulating factors. The autologous skin cells required for such purpose may, for example, be derived from biopsies. Typical fields of application are extensive burns or therapy resistant chronic ulcers. Similar to methods using autologous skin substitutes, it is particular advantageous that no rejection reactions of the immune system occur under these conditions.

The afore described complex mode of action of the DNA binding proteins and in particular of the HMG protein described herein and the nucleic acid coding therefore, which are used in accordance with the present invention, provides for a sufficient blood flow of the wound area and provides for an explanation for their use in the vascularisation in cardiac infarction. Under the influence of the DNA binding proteins and particularly of the HMG proteins described herein and used in accordance with the present invention, there is an induction of the proliferation of endothelial cells which promote angiogenesis in the wound area and provide for a supply of the heart muscle with blood vessels. This is also true for the application in connection with the healing of tooth and bone implants. In connection therewith, also the proliferation promoting effect of the DNA binding proteins and in particular of the HMG proteins described herein and the nucleic acids coding therefore, comes into being including the angiogenesis effect as described above. Finally, the effect that under the influence of the DNA-binding proteins and in particular of the HMG binding proteins described herein which are used in accordance with the present invention, the mobility of the cells is increased, comes particularly into being in this field of application.

An advantageous application of the basic DNA proteins and the nucleic acids according therefore as described herein, is also the field of tissue aging which is slowed down or abolished under the influence of said proteins. A further application is tissue rejuvenation, whereby there are overlaps between tissue aging and tissue rejuvenation with regard to the underlying processes which, however, can be influenced by the basic DNA binding proteins and the nucleic acids coding therefore, as described herein. Without wishing to be bound thereby in the following, it seems that this effect is based on the feature of said DNA binding proteins, to convert the cells into a condition which is very similar to the one of a young differentiated and foetal skin cell, respectively. In this manner, the cells subject to a treatment with said proteins undergo a rejuvenation process and a dedifferentiation process by which the cells are capable to regenerate again. In particular the age caused slowing down of and decrease in cell regeneration, which is, among others, influenced by hormonal changes and hereditary factors, can be counteracted by the basic DNA binding proteins and the nucleic acids coding therefore, respectively. Also damages, which are generated by exogenous factors such as free oxygen radicals, may be decreased, removed or their generation be avoided by an active cell regeneration which is induced by the basic DNA proteins as described herein. Insofar the basic DNA proteins and the nucleic acids coding therefore which are used in accordance with the present invention, allow for a preventive and/or causal and not only a primarily symptomatic therapy which is factually suitable to counteract already existing skin damages by cell regeneration. Furthermore, there is an activation of collagen expression in the process of influencing tissue aging when using said proteins in accordance with the present invention, thereby counteracting a decrease and loss, respectively, of collagen and thus one of the main reasons of wrinkles.

It is within the present invention that instead of DNA binding proteins and in particular the HMG proteins as described herein, also nucleic acids coding for them as described above, may be introduced into the respective cell and the respective tissue. The thus treated nucleic acids are preferably integrated into an expression vector which allows for the expression of the nucleic acid in the respective cell and tissue formed therefrom, so that the corresponding DNA binding proteins and in particularly the HMG proteins described herein, are intracellularly expressed. The respective expression vectors for the individual cells are known to the one skilled in the art and, for example, described in Colosimo A. et al. (2000) Transfer and expression of foreign genes in mammalian cells. Review. Biotechniques 29:314-331, and Sambrook, J., Fritsch, E. F., Maniatis, T. (1989) A Laboratory Manual. CSH Laboratory Press, Cold Spring Harbor, N.Y., respectively. Suitable viral vectors for the expression of genes are, for example, derived from inactivated viruses such as, for example, adenoviruses, adeno-associated virus, Epstein-Barr viruses, Herpes simplex viruses, Papilloma viruses, Polyoma viruses, retroviruses, SV40 and Vaccinia viruses. Suitable plasmid vectors are typically composed of prokaryotic, eukaryotic and/or viral sequences. Examples therefore are in particular pTK2, pHyg, pRSVneo, pACT, pCAT, pCAT-based vectors, pCI, pSI, pCR2.1, pCR2.1-based vectors, pDEST and pDEST-based vectors. Also, methods are known to the one skilled in the art to introduce the respective DNA into said cells and tissue, respectively, whereby the introduction can be in situ, in vivo or in vitro. Respective methods comprise, among others, PO₄-precipitation (CaPO₄, BES—CaPO₄, SRPO4), cationic polymers, liposomes, molecular conjugates (for example polylysine), Gramicidin S-DNA-lipid complexes, electroporation, biolistic gene gun, microinjection, recombinant viruses and naked DNA, such as for example described in Colosimo A. et al. (2000) Transfer and expression of foreign genes in mammalian cells. Review. Biotechniques 29:314-331, as well as Sambrook, J., Fritsch, E. F., Maniatis, T. (1989) A Laboratory Manual. CSH Laboratory Press, Cold Spring Harbor, N.Y.

A similar approach may also be used if it is desired to express a functional nucleic acid as defined herein in a cellular background such as a cell, a tissue or an organ, particularly if the functional nucleic acid inhibits the expression, i.e. transcription and translation, respectively, of the DNA-binding protein described herein and in particular the HMG-protein described herein.

It is thus within the present invention that the use of the DNA-binding proteins as described herein and in particular of the HMG proteins as described herein, can be replaced by a nucleic acid coding therefore which result in the expression of the respective proteins in an expression system. Suitable expression systems are, among others, cell lysates, cells, tissues and/or organs.

In a method according to the present invention, in particular an in vitro method, for angiogenesis or neovascularization of tissue comprising the following steps:
a) providing a tissue or a part thereof,
b) adding one or several nucleic acid(s), the transcription product(s) thereof and/or translation product(s) thereof, and
c) incubating the tissue and the nucleic acid(s), the transcription product(s) thereof and/or the translation product(s) thereof,
   whereby the nucleic acid(s) is/are selected from the group comprising genes for HMG proteins, the DNA binding proteins, in particularly the HMG proteins described herein and the respective nucleic acids can be used.

The DNA binding basic proteins as described herein and the respective nucleic acids can be used in a method in accordance with the present invention, in particular an in vitro method, for the regeneration of tissue comprising the following steps:
a) providing a tissue or a part thereof,
b) adding a nucleic acid, the transcription product thereof and/or the translation product thereof, and
c) incubating the tissue and the nucleic acid, the transcription product thereof and/or the translation product thereof,
   whereby the nucleic acid is selected from the group comprising the genes for basic DNA-binding proteins.

Preferably, the tissue provided is of the kind which is to be regenerated. Nevertheless it is not necessary with regard to the mode of action of the basic DNA-proteins used in accordance with the present invention, that the tissue which is provided and used in this step, is identical to the tissue finally actually obtained. For example, it is possible to generate cartilage cells from fat cells, or muscle cells from cartilage cells under the influence of the HMG genes and the proteins and polypeptides, respectively, described herein. Without wishing to be bound thereto, the present inventor assumes that the cell(s) used as starting cell(s) is/are transformed through a stem cell like condition into the respective cell type by the DNA-binding proteins which are used in accordance with the present invention. Insofar the methods described herein are also methods for the generation of quasi stem cells.

The provision of a tissue can, for example, be made by biopsy or exploitation.

The incubation of the tissue with a nucleic acid, the transcription product thereof and/or the translation product thereof, as described and defined, respectively, herein, into the tissue or a part thereof can be performed such that the translation product and the nucleic acid, respectively, or a transcription product transferred into one or several cells of the tissue. Preferably, the nucleic acid is provided for such purpose in a form which allows the transfection of one or several cells of the tissue. Suitable measures for such purpose are, for example, the addition of the nucleic acid and the transcription product thereof, respectively, as a liposome or incubation of the same together with a liposome or a different form which allows for the transfection of cells with nucleic acids. The translation products, in particular the HMG proteins used in accordance with the present invention, can be introduced into the cell during incubation using methods known in the prior art, such as, for example, electroporation, treatment of the cells with bacteriotoxin such as streptolysin O or protein transduction domains and peptide carriers.

The afore described method of the present invention for the regeneration of tissue may also be used for the repair of DNA damages and for supporting the repair of DNA damages, respectively, in or of cells. In connection therewith, particularly the repair of DNA damages in the epithelialum of the skin is caused and promoted, respectively.

Electroporation, which generates reversible openings (pores) in the membrane by short electrical pulses, were used for the first time in the 70s for introducing foreign molecules in cells. Both low molecular substances (such as dyes and peptides) and high molecular compounds (such as proteins, DNA and RNA) can be introduced into bacterial cells as well as eukaryotic cell through the membrane pores thus formed. As this method, compared to other methods, exhibits a comparatively low transport efficiency, one will preferably use the following methods and agents in clinical applications.

The membranes of eukaryotic cells can be rendered permeable by means of a bacterial toxin such as streptolysine O. The transfer of HMG proteins into eukaryotic cells using streptolysine 0 (SLO) is performed by varying the Ca²⁺ concentration. In the absence of Ca² ions the cells are lysed and the cell pores can be closed again by subsequent addition of Ca²⁺ ions. In order to ensure the reversibility of the lysis process the optimum SLO concentration will be determined for each cell type in the course of routine experiments known to the one skilled in the art. Using this method the drugs disclosed herein, i.e. the HMG proteins and the nucleic acids coding therefor, as described herein, are introduced in, for example, skin cells in order to, for example, stimulate the growth of in vitro cultivated autologous keratinocytes. Highly proliferative cells can thus be provided as transplants to a patient having chronic wounds such as, for example, burn patients.

Liposomes were used for studies on the ion transport through cell membranes for the first time in 1961 and were discovered later to be a suitable means of transportation of medicaments. Although systemic administration of medicaments encapsulated in liposomes has only been of little success, topical application of liposomes offers new chances in the field of dermatology. Additionally, cosmetic products based on liposomes are also marketed in the United States and in Western Europe. Insofar, liposomes represent preferred application forms for the administration of HMG proteins and the nucleic acids coding therefor, respectively, particularly for the manufacture of medicaments and products for external application as described herein.

Liposomes are micelles which have a design similar to the one of lipid double layer of the cell membrane and which will fuse with them upon excessive addition to cells. Therefore, drugs which have previously been added to the hydrophilic phase of the liposomes, and encapsulated drugs, respectively, can be released into the cell. The classification of the liposomes is based on their size and on the number of lipid double layers. The are big vesicles (from 0.1 to >10 μm) having several lipid double layers (multi-lamellar large vesicles=MLV), big vesicles (>0.06 μm) having a single lipid double layer (large uni-lamellar vesicles) and small vesicles (0.02 to 0.05 μm) with a single lipid double layer (small uni-lamellar vesicles). The number of lipid bilayers allows, to a certain extent, the control of the quantitative release or delivery of the drug into the cell. Additionally, the compatibility between liposome and skin may be increased by incorporating, for example, ceramides rather than phospholipid components, i.e. structures which are similar to the membrane structures of keratinocytes. This method is thus particularly suitable for a preparation which is to be applied topically and which contains a drug on the basis of the HMG proteins and the nucleic acids coding therefore as described herein. Due to the small size of the HMG proteins, in particular of the HMGA proteins (<12 kDa) they are additionally particularly suitable for packaging in liposomes.

Protein transduction domains (PTD) and peptide carriers represent an efficient possibility to introduce into cells the proteins which are to be used in accordance with the present invention. PTDs are in general short peptides comprising 10 to 16 amino acids, usually positively charged lysine and arginine residues which are covalently linked to the protein to be transported. PTD mediated transduction occurs through a to date hardly known mechanism which is independent of receptors, transporters and endocytosis. Using PTDs proteins having a size of up to 700 kDa could be introduced into cells. Additionally, PTDs are particularly suitable for the transport of drugs of medicaments such as the herein described HMG proteins and the nucleic acids coding for them, used in accordance with the present invention, as in vivo transduction of proteins could already be detected in tissue and cells. Due to the covalent binding of the PTDs to the proteins to be transferred, however, this technology is limited with regard to the requirement that the functionality of the drug to be transported has to be maintained. Preferably also for that reason non-covalent peptide carriers are used in a preferred embodiment, such as the chariot reagent (Carlsbad). This protein transport system is based on a short synthetic signal peptide (Pep-1), which complexes with the protein to be transported by non-covalent hydrophobic interaction. Within the cells the transported protein dissociates from the Pep-1 peptide and is transported to the intended intracellular location by means of cellular transport mechanisms. A further advantage of this method is its high transportation efficiency which is, depending on the cell type and protein, between 60 to 95%. This method is thus suitable for use in connection with the promotion of proliferation which is mediated through HMG proteins for in vitro cultivated autologous skin cells, as well as in connection with a preparation which is to be topically administered, containing one or several of the drugs described herein.

The incubation of the tissue with the nucleic acid, the transcription product thereof and/or the translation product thereof is performed under conditions which allow the uptake of the same into the cell and tissue, respectively. Preferably the incubation occurs at 37° C. under physiological conditions.

In a further aspect the present invention is related to a method, in particular an in vitro method, for the differentiation, dedifferentiation and/or reprogramming of cells comprising the steps:
a) providing one or several cells,
b) providing a nucleic acid, the transcription product thereof and/or a translation product thereof, and
c) incubating the cell and the nucleic acid, the transcription product thereof and/or the translation product thereof, whereby the nucleic acid, the transcription product thereof and/or the translation product thereof can be embodied as described in connection with any of the other aspects as described above.

What has been said in connection with performing the individual steps of the method for the regeneration of tissue as described herein, applies also to this method. Although not limited thereto, the cell which is provided can be any cell, in particular any mesenchymal cell such as a fat cell, a cartilage cell or a muscle cell.

In a further aspect the present invention is related to a pharmaceutical composition which comprises one or several nucleic acid(s), the transcription product(s) thereof, the translation product(s) thereof, the functional nuclei acids and the compounds identified by applying the screening methods in accordance with the present invention, as described herein, and a pharmaceutically suitable carrier. A pharmaceutical composition can be a composition which is embodied for the various forms of applications. Such forms of application include in particular topical application and subcutaneous application. The same is also true for the cosmetic preparation according to the present invention. Suitable pharmaceutical carriers as well as cosmetic carriers are, effective for the Federal Republic of Germany, as defined, for example, in the regulation on cosmetic means dated Jun. 19, 1995, and can comprise the following ones individually or in any combination: buffers, stabilizers, bacteriostatics, alcohols, bases, acids, starch, moisturizers, creams, fatty ointments, emulsions (oil in water (O/W); water in oil (W/O); water in oil in water (W/O/W)), microemulsions, modified emulsions, nanoparticles/nanoemulsions, liposomes, hydrodispersion gels (hydrogels, alcohol gels, lipogels, tenside gels), gel-creams, lotions, oils/oil baths and sprays.

The pharmaceutical compositions according to the present invention may comprise further drugs apart from the DNA binding proteins used in accordance with the present invention, in particular the HMG proteins described herein, and the nucleic acid(s) coding therefor, or the inhibitory nucleic acids derived from the nucleic acid sequence of these proteins such as antisense nucleic acids, ribozymes or RNAi. Pharmaceutical or cosmetic formulations comprising at least one HMG protein and a nucleic acid coding therefor, respectively, are preferably ointments, creams and gels.

The present invention is further based on the surprising finding that the DNA binding proteins as described herein, in particular the HMG proteins as described herein, show a spontaneous transfer into animal cells, in particular epithelial cells and more particularly into human epithelial cells. Because of this it is possible that said proteins are immediately applied to a cell covered surface, in particular onto the cells to be treated, which subsequently take up said proteins. Preferably the proteins are contained in a carrier medium which promotes this spontaneous transfer. Such transfer media are, for example, aqueous or alcoholic solutions or suitable emulsions or other phases or mixtures of phases. This surprising behaviour of the DNA proteins described herein, in particular of the HMG proteins, provides for an immediate use thereof in pharmaceutical and/or cosmetic formulations, whereby no further particular measures are required such as, for example, the use of streptolysine for the uptake of the proteins into the cell to be treated.

Using the nucleic acid(s) according to the present invention, the transcription product(s) thereof and/or the translation product(s) thereof as well as the functional nucleic acids disclosed herein and the compounds identified by applying the screening methods in accordance with the present invention, and the pharmaceutical compositions prepared therewith, any application method known in the prior art may be used. For example, intradermal, subcutaneous, intramuscular or intravenous and intra-arterial application may be performed by using injection syringes, and application can be realised directly into the target tissue, respectively. Also, catheter probes or direct application onto a freely accessible target tissue may be used. The corresponding application method which will be used, will normally be determined by the target tissue. If, for example, a revascularization of cardiac muscle tissue is intended, a formulation according to the present invention will preferably be made by means of a catheter, needles or a combination instrument which, for example, also allows the application of laser pulses in connection with TMLR. If, for example, skin tissue is to be targeted, for example in order to promote angiogenesis by means of the nucleic acids according to the present invention, the transcription products thereof or the translation products thereof such that wound healing is improved, or in order to inhibit angiogenesis, for example, in case of haemangioma or small varicose veins by means of inhibitory molecules derived from the nucleic acids according to the present invention such as antisense nucleic acids, ribozymes or RNAi, or inhibitory substances identified in the screening methods, then, for example, an intradermal or subcutaneous administration or also topical administration as creams may be appropriate. It is within the skills of the ones of the art to select suitable administration methods.

In a further aspect the present invention is related to cells which may be obtained by a method in accordance with the present invention, as well as tissues which may be obtained by the methods according to the present invention.

In a further aspect the invention is related to carrier material which comprises one or several nucleic acid(s), the transcription product(s) thereof, the translation product(s) thereof, one or several of the functional nucleic acids described herein and/or one or several of the compounds identified by applying the screening methods in accordance with the present invention, whereby the nucleic acid(s), the transcription product(s) thereof and/or the translation product(s) thereof may be those as described herein. The carrier material is used in particular as an implant or as cover material, preferably for wound healing, but also for each and any of the other diseases or conditions described herein or the other applications described herein, which comprise the provision of a surface to or onto which the basic DNA binding proteins as described herein are bound in a covalent or non-covalent manner. In principle, any materials can be used for such purpose which are used for implant materials or as cover materials and carrier materials, respectively, for wound healing or the other applications described herein, which are related to the provision of a surface to or onto which the DNA binding proteins, in particular HMG proteins as described herein, are or will be covalently or non-covalently bound, and which are known in the art, including, but not limited to, hydrocolloids, hydrogels, hydropolymers, foam dressings, calcium alginate, actived carbon, foamed plastic, sheets, silicone foams, fleece material, man-made continuous filaments, cotton gauze, rubber, paraffin and paraffin gauze. Suitable plastics are polyethylenes, polyvinylenes, polyamides and polyurethanes. The nucleic acids and DNA binding proteins used in accordance with the present invention, in particular the HMG proteins, are preferably absorbed by the carrier material in a non-covalent manner. However, it is also within the present invention that they are covalently bound and/or a release thereof from the actual carrier material occurs under the respective application conditions. Suitable potential applications are known to the one skilled in the art. A particular form of the carrier material is the wound covering material consisting of a basic cover material and one or several nucleic acid(s), the transcription product(s) and/or the translation product(s) thereof, whereby these are embodied as described herein. In this case the basic cover material may form a carrier material in the meaning described herein.

In a further aspect the present invention is related to a method for the screening of a compound, whereby the compound promotes and/or inhibits one process. The process can be each and any of the processes, individual or in any combination, as described herein, more particularly the process can be selected from the group comprising tissue regeneration, wound healing, cell mobility, repair of skin damages, angiogenesis, angiogenesis in the wound area, epithelialization, tissue aging, changes in tissue aging, tissue rejuvenation, vascularization, neovascularization, vascularization in connection with cardiac infarction and healing of tooth and bone implants. Additionally, the process can generally be any process comprising reprogramming, redifferentiation or dedifferentiation, optionally with subsequent new differentiation. Without wishing to be bound in the following it seems that the processes as described herein are associated with a transformation of at least one cell into a condition which is a quasi stem cell character based on which or starting from which a new differentiation of the cell occurs.

In the easiest form the method for the screening of a compound in accordance with the present invention comprises the following steps:
a) providing a test system for the process;
b) providing a candidate compound; and
c) testing of the candidate compound and determining the reaction caused by the candidate compound in the system.

The test system is preferably a system which allows to represent the respective process, in particular to present the process under the influence of a compound which is thought that it either promotes or inhibits said process, which is a so-called candidate compound, and/or under the influence of a reference compound. Such systems are known to the one skilled in the art. Preferably such a test system comprises one or several cells, optionally a tissue or tissues containing said cell(s), whereby the behaviour of the cells and the tissues, respectively, is analysed. In connection therewith, the behaviour is one or several of the following processes: growth of the cells and the tissue, respectively, differentiation of the cell and the tissue, respectively, and the various facets and aspects thereof such as, for example, but not limited thereto, dedifferentiation and differentiation, preferably new differentiation, motility of cells, release of signal molecules, angiogenesis or neovascularization of tissues. Apart from direct growth other phenomena or parameters may be used in order to describe a reaction of the test system. Such parameter may be, for example, biochemical, genetic, moleculargenetic, molecularbiological, histological, cytological, physiological and phenotypic parameters. Biochemical parameters may, for example, be metabolic pathways, starting agents as well as products thereof which are either directly or indirectly linked to said processes. Genetic and moleculargenetic parameters are preferably those which are associated with said processes at the level of the nucleic acid, both genomic nucleic acid as well as hnRNA, mRNA and the like. It may be within the present invention that the presence of a respective nucleic acid is measures as genetic parameter, the disappearance of a respective nucleic acid, or the quantitative changes thereof upon promotion or inhibition of said processes. The molecular parameters can be associated, among others, with the proteins and their appearance and disappearance in said processes. Physiological parameters may be the behaviour, in particular the response behaviour of the cells and the tissue in response to stimuli such as, for example, biological, chemical or physical stimuli to which the respective cell system, i.e. the cells and the tissue, depending on the process and its promotion and inhibition, respectively, responds in a different manner.

In an embodiment of the method for the screening of a compound for promoting and/or inhibiting such processes in accordance with the present invention it is contemplated that apart from the respective test system for the process a reference compound is provided and the reference compound is contacted with the test system, i.e. the reference compound is tested in the test system. This contacting occurs preferably such that the reference compound, preferably present in a solution, more preferably a buffer, is contacted with the test compound, to which preferably culture medium is added. It is also within the present invention that the contacting of the reference compound with the test system occurs in a site-specific manner, for example that the reference compound is incorporated into distinct cells of the tissue or also into distinct compartments of the cell(s). The cell-specific as well as compartment-specific delivery of such reference compounds is basically known to the one skilled in the art. It is possible that, for example, reference compounds are injected in defined areas and compartments, respectively, of the cell by microinjection as, for example, described in Wang B et al (2001) Expression of a reporter gene after microinjection of mammalian artificial chromosomes into pronuclei of bovine zygotes. Mol Reprod Dev 60:433-8. Additionally, methods are available for the treatment of the reference compound, for example by amino acid transporters, or by modifying them such that the reference compounds reach distinct cells such as, for example, fibroblasts, as, for example, described in Palacin M et al. (1998) Molecular biology of mammalian plasma membrane amino acid transporters. Physiol Rev 78:969-1054, or specific cell compartments as, for example, the nucleus, as, for example, described in Chaloin L et al. (1998) Design of carrier peptide-oligonucleotide conjugates with rapid membrane translocation and nuclear localization properties. Biochem Biophys Res Commun 243:601-608, or the mitochondria as, for example, described in Pain D et al. (1991) Machinery for protein import into chloroplasts and mitochondria. Genet Eng (N Y) 13:153-166. The cell-specific or compartment-specific delivery can also be practised for the candidate compound as described herein.

In a subsequent step, the reaction caused by the reference compound in the test system is determined. In connection therewith, preferably, the aforementioned parameters may be used in order to determine the impact of the reference compound in the test system. In a further step of this embodiment of the method in accordance with the present invention for the screening of a compound for promoting and/or inhibiting one of said processes, the candidate compound is then provided and, similar to the reference compound, tested in the test system. Subsequently, the reaction caused by the candidate compound in the test system is determined, whereby basically the parameter described in connection with the reference compound, are used. Finally, the reaction of the test system, represented by the aforementioned parameters, under the influence of the reference compound, is compared to the reaction of the test system under the influence of the candidate compound in the test system. A candidate compound is referred to as a compound for promoting one or several of said processes provided that the respective process generates the same reaction or a stronger reaction in the test system under the influence of the candidate compound compared to the reference compound. On the other hand, a candidate compound is a compound for inhibiting one or several of said processes provided that the candidate compound causes a reaction in the test system which is less pronounced than the respective reaction of the referenced compound. It is within the present invention that the same candidate compound may have different effects in the sense of inhibition and promotion, respectively, of a process compared to a different one of the aforementioned processes. It is further within the present invention that the time course of the testing of the reference compound and the candidate compound can be reversed.

In a further aspect of the method in accordance with the present invention for the screening of a compound for promoting and/or inhibiting one of said processes, again a test system is provided for said process and, subsequently, a reference compound. In this embodiment the reference compound is provided with a label. In principle, any labels are suitable, particularly those which comprise a radioactive, fluorescent, immunological, enzyme or affinity label and allows such label, respectively. Radioactive labels are particularly ¹H, ³H, ³⁵S, ³²P, ³³P, ¹²⁵I, ⁵¹Cr, ¹³C and ¹⁴C, fluorescence labels comprise labels using fluorescein, fluorescamine, isocyanate, luciferase, rhodamine, Texas Red, Cy3 and Cy5. The immunological labels are diverse immunogens, among others, the immunoglobulins IgM, IgA, IgD, IgE and IgG, including, but not limited thereto, IgG1, IgG2a and IgG2b. Enzyme label comprise in particular alkaline phosphatase and peroxidase. Affinity labels are GST and His-tag labels as well as any label by means of biotin and digoxygenin. Preferably, the label will be one which does not interfere with the reaction caused by the reference compound in the test system. Such a labelled reference compound is, as described above, subsequently tested in the test system and the reaction caused in the test system determined. In a further step the candidate compound will subsequently be provided and also tested in the test system as described above, whereby the test system contains the reference compound during the testing of the reaction caused by the candidate compound. It is preferred that the testing occurs under conditions which ensure that the reference compound is still biologically active, i.e. shows a promoting and inhibiting, respectively, effect. After addition of the candidate compound the reaction of the test system is determined again, whereby it is in principle possible that the afore-described biochemical parameters are used. Preferably, the amount of released reference compound is additionally or alternatively determined by means of the respective label or the amount of the released label as such.

In a still further aspect of the method for the screening of a compound for promoting and/or inhibiting one of said processes in accordance with the present invention, it is contemplated that the candidate compound is labelled. In an embodiment the candidate compound is provided and subsequently tested in the test system and the reaction caused by the candidate compound in the test system is determined with subsequent provision of a reference compound, followed by testing of the reference compound in the test system, whereby the test system contains the candidate compound, in particular under conditions which allow that the reference compound is physiologically active, and the reaction of the test system is determined, whereby the amount of released candidate compound and/or released label of the candidate compound is determined. Alternatively, but also in addition thereto, said parameters may be used as described above, in order to characterise the reaction of both the reference compound and the candidate compound on the respective process. It is also within the present invention that the sequence of the addition of the candidate compound and the reference compound, independent which of the compounds is provided with a label, is reversed. In the first of said two afore-described procedures the reference compound competes with the candidate compound, in the second case the candidate compound competes with the reference compound. Finally, it is within the present invention that in the various aspects of the screening method according to the present invention, where either the candidate compound or the reference compound comprises a label, which is also referred to herein as first label, also the other compound comprises a label which is in the following also referred to as second label, whereby the first and the second label are preferably different from each other.

In connection with any of the methods for the screening of a compound for the promotion and/or inhibition of one of said processes according to the present invention, it is contemplated that the reference compound is a nucleic acid, the transcription product thereof or the translation product thereof, optionally a combination thereof, whereby the nucleic acid is selected from the group comprising genes for DNA binding proteins and HMG proteins. Particularly preferred DNA binding proteins and HMG proteins are those as described herein, and in particular those according to SEQ ID NO. 1 to SEQ ID NO: 30, and the nucleic acids coding for proteins according to SEQ ID No. 31 to 64 as well as those depicted in tables 1 and 2, respectively.

It is within the present invention that during the various applications and uses a single one of the DNA binding proteins and of the HMG proteins and/or of the nucleic acid coding for a single one or for one of the DNA binding proteins and HMG proteins described herein, is used. However, it is also within the present invention that a mixture of two or several of said proteins and of the nucleic acids coding therefor, respectively, may be used. Furthermore, it is within the present invention that the terms protein and peptide and polypeptide, respectively, are used in an interchangeable manner herein.

A further aspect of the present invention is related to the use of DNA binding proteins, particularly HMG proteins, and of the nucleic acids coding therefor and preferably of molecules interacting with them, which preferably antagonize them, in order to inhibit distinct biological processes. Based on this inhibition, prevention or treatment of diseases is possible which are associated with these processes in a causal or symptomatic manner. Such diseases comprise, however, are not limited thereto, tumors, tumor diseases, histiocytosis, inflammatory diseases, inflammation and arthritis, which are also referred to herein as the “aforementioned diseases”. The aforementioned diseases may be respective diseases of man as well as respective diseases of animals, in particular pets, zoo animals, farm animals and the like. DNA binding proteins and in particular HMG proteins as discussed in connection with this and other aspects, comprise all such proteins, in particular the HMG proteins, HMG peptides and fragments thereof described herein, as well as the nucleic acids coding therefor. If not explicitly mentioned herein differently, proteins also refer polypeptides and vice versa. In particular, the term HMG proteins comprises also HMG peptides and fragments thereof.

Due to this causal relationship a further aspect of the present invention is related to agents for the prevention and/or treatment of any of the aforementioned diseases. Such agents are preferably those which inhibit and antagonize, respectively, the effect of the DNA binding proteins, in particular of the HMG proteins, and the nucleic acids coding therefor. Respective agents are polypeptides binding to DNA binding proteins, in particular to the HMG proteins, or the nucleic acids coding therefor as well as antibodies binding to DNA binding proteins, in particular HMG proteins, or the nucleic acids coding therefor. Further agents which may be used for the prevention or treatment of these diseases are siRNA or RNAi, aptamers, spiegelmers, antisense molecules and ribozymes. Insofar the present invention is related to the use of such agents for the manufacture of medicaments, in particular medicaments for the treatment of any of the aforementioned diseases.

The aforementioned agents may be produced by the ones skilled in the art. Any of the DNA binding proteins, in particular HMG proteins, fragment thereof or any nucleic acid coding for the protein or fragment thereof, in particular as disclosed herein, is the basis for such manufacture. It is particularly preferred that the protein is HMGB1 and parts thereof, in particular the A domain of HMGB 1. The agents according to the present invention which may be used in accordance with the present invention for the manufacture of medicaments for the treatment and/or prevention of any of the aforementioned diseases, is thus an antibody against DNA binding proteins, in particular HMG proteins, or fragments thereof, peptides binding to DNA binding proteins, in particular HMG proteins or fragments thereof, siRNA against mRNA of DNA binding proteins, in particular of HMG proteins or fragments thereof, antisense molecules directed against nucleic acids coding for DNA binding proteins, in particular HMG proteins or fragments thereof, whereby the nucleic acid is in particular mRNA or hnRNA, ribozymes directed against nucleic acids coding for DNA binding proteins, in particular HMG proteins or fragments thereof, in particular mRNA or hnRNA. Furthermore, the means are aptamers and spiegelmers directed against DNA binding proteins, in particular a HMG protein or fragment thereof, or a nucleic acid coding therefor.

Antibodies as used herein are preferably monoclonal antibodies which may be generated according to the protocol of Cesar and Milstein and further developments thereof. Antibodies are also antibody fragments or derivatives such as, for example, Fab fragments, Fc fragments, but also single-stranded antibodies provided that they are in principle capable of specifically binding to HMGB. Apart from monoclonal antibodies also polyclonal antibodies may be used. A polyclonal antibody for basic research which could, in principle, also be used as a medicament, is, for example, the antibody sc-12523 which is directed against HMGB1 (Santa Cruz Biotechnology, Santa Cruz, USA). Preferably, the used antibodies are human or humanized antibodies.

Peptides or polypeptides which interact with a DNA binding protein, in particular a HMG protein, or a nucleic acid coding therefor, may be screened using methods known in the prior art such as, for example, phage display. These techniques are known to the ones skilled in the art. In connection with the generation of such peptides, usually a peptide library is generated, for example in the form of phages, and this library is contacted with the target molecule, i.e. a DNA binding protein, in particular a HMG protein, preferably with HMGB1. Subsequently, the binding peptides are typically removed as a complex together with the target molecule from the non-binding members of the library. It is within the skills of those of the art that the binding characteristics depend, at least to a certain extent, on the experimental conditions, such as the salt concentration and the like. After separating the peptides binding with a higher affinity or with a bigger force to the target molecule, from the non-binding members of the library and from the target molecule, respectively, they may subsequently be characterised. Optionally, prior to the characterisation an amplification step is necessary, for example by propagating the phages coding for the respective peptide and peptides, respectively. The characterisation preferably comprises the sequencing of the peptides binding to the respective DNA binding proteins, in particular the respective HMG. The peptides are in principle not limited with regard to their length. Typically, however, peptides having a length from 8 to 20 amino acids are used and obtained, respectively, in such methods. The size of the libraries is from 10² to 10¹⁸, preferably 10⁸ to 10¹⁵ different peptides.

A particular form of target molecule binding polypeptides are anticalines, as, for example, described in the German patent application DE 197 42 706.

Additionally, also small molecules may be used which antagonize the effect of the DNA binding proteins, in particular of the HMG proteins, and the nucleic acid coding therefor. Such small molecules may, for example, be identified by means of a screening method, in particular a screening of libraries of small molecules. In connection therewith, the target molecule is contacted with the library and those members of the library binding thereto are determined, optionally separated from the other members of the library and the target, respectively, and also optionally further characterised. Again, the characterisation of the small molecule is performed in accordance with procedures known to the ones skilled in the art, for example, the compound is identified and the molecule structure is determined. These libraries comprise as little as two and as many as several 100 000 members. Aptamers, as used herein, are D nucleic acids, either single-stranded or double-stranded, which specifically bind to a target molecule. The generation of aptamers is, for example, described in European patent EP 0 533 838. In connection therewith it is proceeded as follows:

In the method for the generation of aptamers a mixture of nucleic acids, i.e. potential aptamers, is provided, whereby any of said nucleic acids consists of a segment of at least eight subsequent randomised nucleotides, and this mixture is contacted with DNA binding proteins, in particular HMG proteins, nucleic acids coding therefor, interaction partners of DNA binding proteins, HMG interaction partners, in particular the natural interaction partners and/or the nucleic acid coding therefor, whereby nucleic acids binding to the target, optionally on the basis of an increased affinity, are compared to the candidate mixture, are separated from the candidate mixture and the thus obtained nucleic acids binding to the target, optionally with a high affinity or bigger force, are amplified. These steps are repeated several times so that after completion of the method nucleic acids specifically binding to the respective target, so-called aptamers, are obtained. It is within the present invention that these aptamers may be stabilised, for example by introducing distinct chemical groups as known to the ones skilled in the art of aptamer development. Aptamers are already therapeutically used. It is also within the present invention that the aptamers thus generated are used for target validation and as lead compounds for the development of medicaments, in particular of small molecules.

The manufacture or generation of spiegelmers is based on a basically similar principle, whereby the spiegelmers can be generated in accordance with the present invention using as target molecules the DNA binding proteins, in particular HMG proteins, the nucleic acids coding therefor, the interaction partners of DNA binding proteins, the interaction partners of HMG, in particular the natural interaction partners, and/or the nucleic acids coding therefor. The generation of spiegelmers is, for example, described in international patent application WO 98/08856. Spiegelmers are L-nucleic acids, i.e. they consist of L-nucleotides, and are essentially characterised by the fact that they exhibit a very high stability in biological systems and because of this, similar to aptamers, specifically interact with a target molecule and bind thereto, respectively. More particularly, a heterogeneous population of D nucleic acids is generated, the population is contacted with the optical antipode of the target molecule, in the present case thus with the D-enantiomer of the naturally occurring L-enantiomer, subsequently those D-nucleic acids are separated which do not interact with the optical antipode of the target molecule, the D-nucleic acids which interact with the optical antipode of the target molecule, are determined, optionally separated and sequenced and subsequently L-nucleic acids are synthesised which are identical in sequence to the one previously determined for the D-nucleic acids. Similar to the process for the manufacture of aptamers it is also possible to enrich and generate, respectively, appropriate nucleic acids, i.e. spiegelmers, by repeating these steps several times.

A further class of compounds which may be manufactured and developed, respectively, using the DNA binding proteins and in particular the HMG proteins and the nucleic acids coding therefor, are ribozymes, antisense oligonucleotides and RNAi.

All these classes have in common that they are not effective at the level of the translation product, i.e. at the level of the DNA binding protein, in particular HMG protein and interaction partners thereof, in particular HMGB1, but at the level of the nucleic acid coding for the respective protein, in particular the mRNA coding for HMGB1.

Ribozymes are catalytically active nucleic acids, which preferably consist of RNA and comprise two moieties. The first moiety is responsible for the catalytic activity, whereas the second moiety is responsible for specific interaction with the target nucleic acid. Upon interaction between the target nucleic acid and the second moiety of the ribozyme, typically by hybridisation of base stretches which are essentially complementary to each other, the catalytic moiety of the ribozyme may either intramolecularly or intermolecularly hydrolyse the target nucleic acid, whereby the latter form is preferred provided that the catalytic activity of the ribozyme is a phosphodiesterase activity. Subsequently, there is an—optionally further-degradation of the coding nucleic acid, whereby the titre of the target molecule is reduced both at the nucleic acid as well as the protein level both intracellularly as well as extracellularly and thus a therapeutic approach for the treatment of the endometrium is provided. Ribozymes, their use and their construction principles are known to the ones skilled in the art and, for example, described in Doherty and Doudna (Ribozyme structures and mechanisms. Annu Rev Biophys Biomol Struct 2001; 30:457-75) and Lewin and Hauswirth (Ribozyme gene therapy: applications for molecular medicine. Trends Mol Med 2001, 7:221-8).

The use of antisense oligonucleotides for the manufacture of a medicament and diagnostic agent, respectively, is based on a principally similar mode of action. Antisense oligonucleotides typically hybridise due to base complementarity with a target RNA, normally with mRNA, and thus activate RNAaseH. RNAaseH is activated by both phosphodiester as well as phosphorothioate coupled DNA. Phosphodiester coupled DNA, however, is rapidly degraded by cellular nucleases with the exception of phosphorothioate coupled DNA. These resistant, non-naturally occurring DNA derivatives do not inhibit RNAaseH, if hybridised to RNA. In other words, antisense polynucleotides are only active as a DNA-RNA hybrid complex. Examples for such antisense oligonucleotides are, for example, described in U.S. Pat. No. 5,849,902 or U.S. Pat. No. 5,989,912. In principle, the essential concept of antisense oligonucleotides resides in providing a nucleic acid which is complementary to a distinct RNA. In other words, starting from the knowledge of the nucleic acid sequence of the HMG proteins and its interaction partners, in particular the respective mRNA, antisense oligonucleotides can be generated by base complementarity which result in degradation of the coding nucleic acid, in particular of mRNA.

A further class of compounds which is in principle suitable as a medicament and diagnostic agent, respectively, is the so-called RNAi. RNAi is a double-stranded RNA which mediates RNA interference and typically has a length of about 21 to 23 nucleotides. In connection therewith, one of said two strands corresponds to a sequence of a gene to be degraded. In other words, starting from the knowledge of the nucleic acid sequence coding for the DNA binding protein, in particular the HMG protein and/or the interaction partners thereof, whereby the nucleic acid is in particular mRNA, a double-stranded RNA can be manufactured whereby one of said RNA strands is complementary to said nucleic acid, in particular mRNA, coding for the DNA binding protein, in particular for the HMG and/or the interaction partners thereof, and this will subsequently result in the degradation of the respective coding nucleic acid and a concomitantly occurring decrease in the titre of the respective proteins. The generation and use of RNAi as a medicament and diagnostic agent is, for example, described in international patent application WO 00/44895 and WO 01/75164.

With regard to the mode of action of the afore-described classes, i.e. ribozymes, antisense oligonucleotides as well as RNAi, it is thus within the present invention to use, apart from DNA binding proteins, in particular HMG proteins, more particularly HMGB1, and the particularly naturally occurring interaction partners, also the coding nucleic acids, in particular mRNA, for the manufacture of a medicament for the treatment and/or prevention of the aforementioned diseases, namely tumors, tumor diseases, histiocytosis, inflammatory diseases, inflammation and arthritis, and to use them for the manufacture of a diagnostic agent for the diagnosis of the aforementioned diseases as well as for monitoring the progress of the disease and the therapy used, either directly or as a target molecule.

It is within the present invention that the aforementioned classes of compounds, i.e. antibodies, peptides, anticalines, small molecules, aptamers, spiegelmers, ribozymes, antisense oligonucleotides as well as RNAi which are directed against DNA binding proteins, in particular HMG proteins, and fragments thereof, or nucleic acids coding therefor and which preferably antagonize the effect of these proteins and the nucleic acids coding therefor, are used for the manufacture of a medicament for the treatment and/or prevention, in particular of tumors, tumor diseases, histiocytosis, inflammatory diseases, inflammation and arthritis.

It is within the present invention that these medicaments and agents, respectively, are not only used for the treatment of the aforementioned diseases, but are also used for diagnostic purposes, i.e. the aforementioned agents may also be used as diagnostics or diagnostic agents, preferably for tumors, tumor diseases, histiocytosis, inflammatory diseases, inflammation and arthritis.

The pharmaceutical or diagnostic compositions containing these agents comprise, in an embodiment, apart from one or several of the aforementioned compounds and of the compounds generated as disclosed herein, additionally other pharmaceutically or diagnostically active compounds or agents, as well as preferably at least one pharmaceutically acceptable carrier. Such carriers may be, for example, liquid or solid, for example a solution, a buffer, an alcoholic solution or the like. Starch and the like are, for example, contemplated as suitable solid carries. It is known to the ones skilled in the art of pharmaceutical formulations how the respective compounds of the various classes have to be formulated so that they may be administered using the desired route of administration, such as, for example, oral, parenteral, subcutaneous, intravenous administration or the like.

Apart from the afore-described agents, which antagonize DNA binding proteins, in particular HMG proteins, fragments thereof and nucleic acids coding therefor, i.e. small molecules, peptides, anticalines, antibodies, aptamers, spiegelmers, ribozymes, antisense oligonucleotides (herein also referred to as antisense molecules) as well as RNAi, also the cell surface receptor RAGE or fragments thereof may be used for such purpose, i.e. as an agent for the manufacture of a diagnostic agent for tumors, tumor diseases, histiocytosis, inflammatory diseases, inflammation and arthritis or of a medicament for the treatment and/or prevention of tumors, tumor diseases, histiocytosis, inflammatory diseases, inflammation and arthritis. The receptor and the fragments derived therefrom, respectively, may, without wishing to be bound thereon in the following, antagonize the effect of the HMG protein, in particular the one of the HMGB1 protein, preferably due to competitive inhibition. The use in accordance with the present invention of the cell surface receptor RAGE and its fragments, and the nucleic acids coding therefor, respectively, for the manufacture of a medicament or a diagnostic agent for the treatment of the aforementioned diseases, results therefrom.

This mode of action is based on the fact that DNA binding proteins, in particular HMG proteins, more particularly HMGB1, are an extracellular ligand to the cell surface receptor RAGE (receptor for advanced glycation end products), which is, for example, described by Taguchi et al. (Taguchi, A., Blood, D. C., del Toro, G., Canet, A., Lee, D. C., Qu, W., Tanji, N., Lu, Y., Lalla, E., Fu, C., Hofmann, M. A., Kislinger, T., Ingram, M., Lu, A., Tanaka, H., Hori, O., Ogawa, S. Stem, D. M., Schmidt, A. M. (2000). Nature 405:354-360).

It is within the present invention that the HMG proteins to which it is referred to herein, are particularly those as disclosed herein. However, they are not limited thereto. Furthermore, it is within the present invention that the term DNA binding proteins is used in an interchangeable manner with the term basic DNA protein.

The present invention is illustrated in the following by reference to the figures, examples as well as the sequence protocol from which further features, embodiments and advantages may be taken.

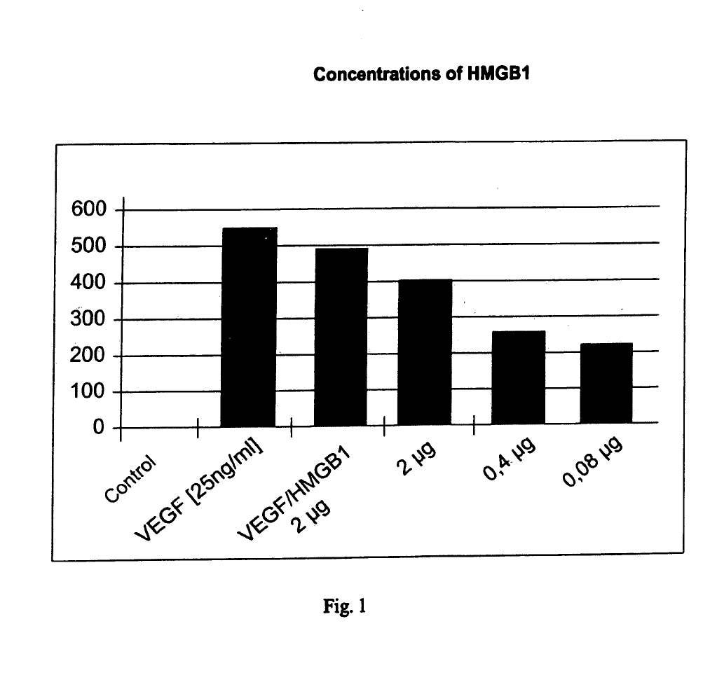

FIG. 1 is a diagram indicating the length of sprouts from spheroids after treatment with VEGF or HMGB1 protein;

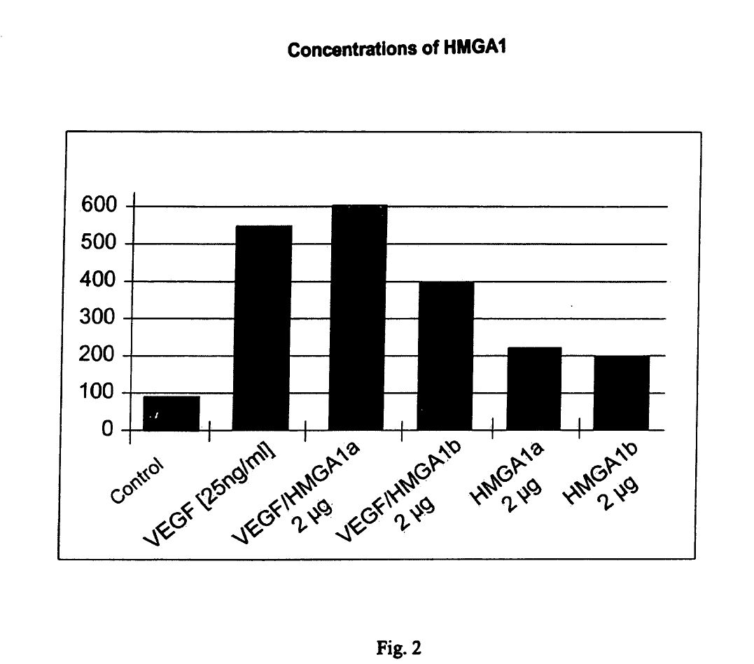

FIG. 2 is a diagram indicating the length of sprouts from spheroids after treatment with VEGF or HMGA 1 protein;

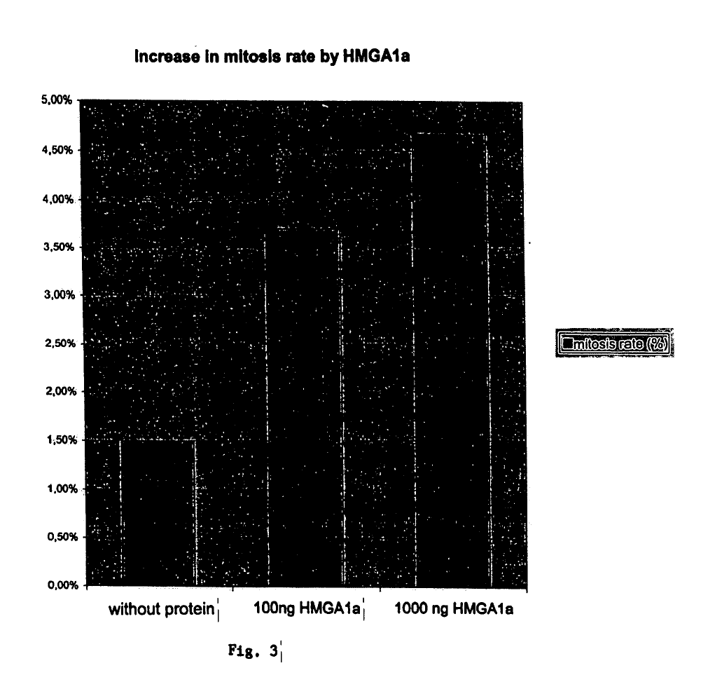

FIG. 3 is an increase in the amount of mitotically active skin cells under the influence of HMGA1a;

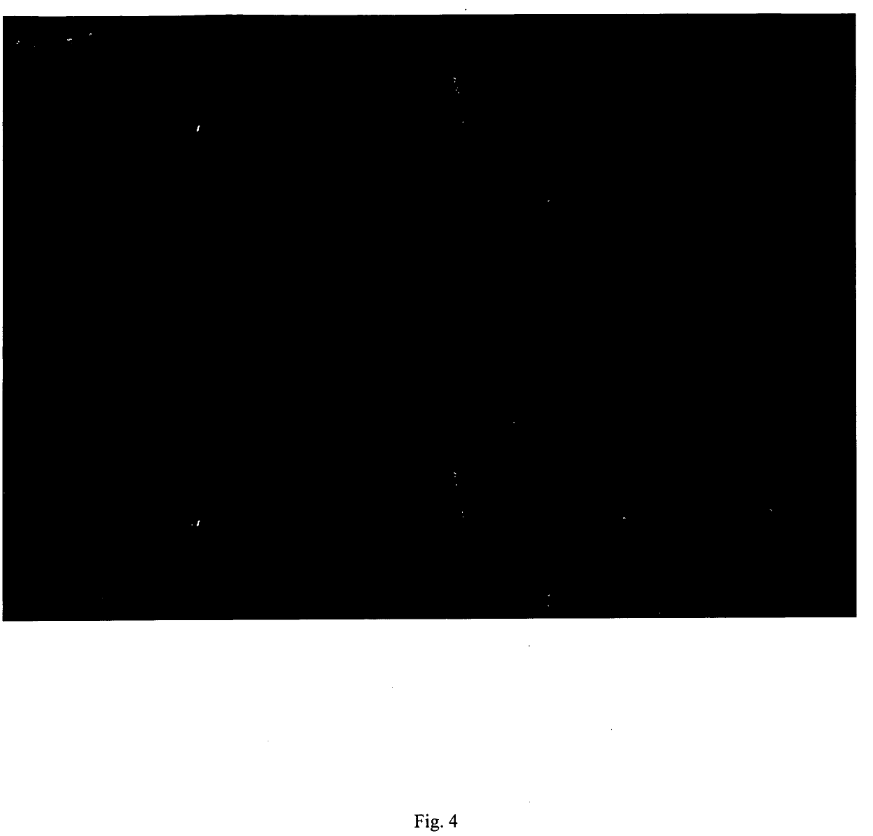

FIG. 4 is an immunofluorescence photograph of Hela cells some of which exhibit fluorescence labelled HMGA1b protein in the cell nuclei;

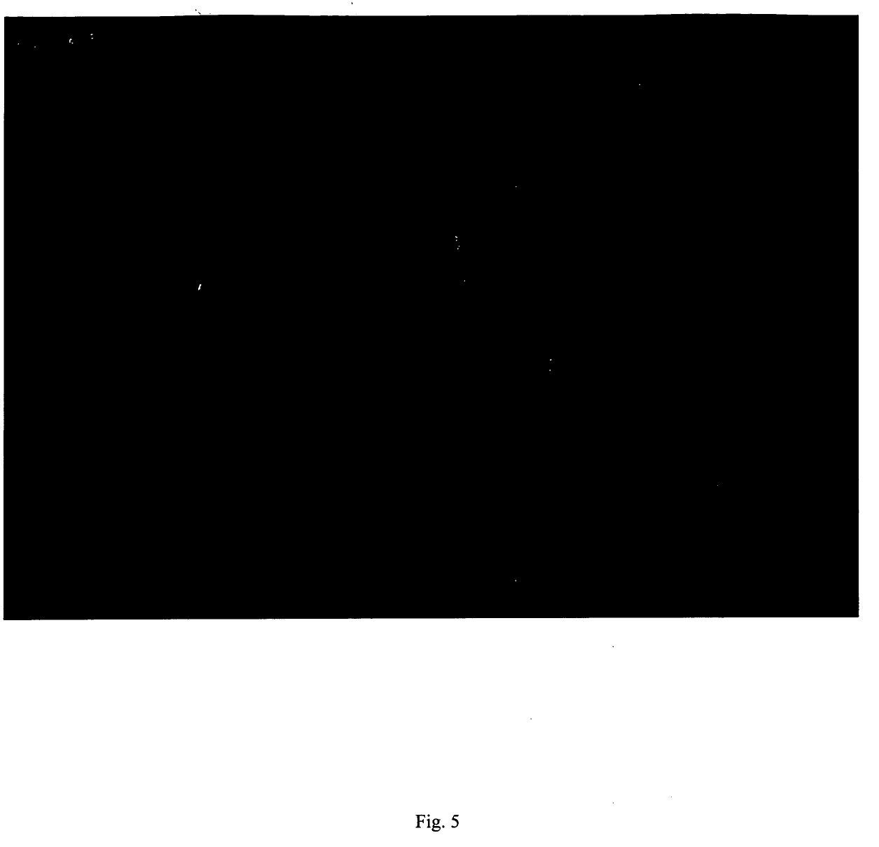

FIG. 5 is an immunofluorescence photograph of fibroblasts some of which exhibit fluorescence labelled HMGA1b protein in the cell nuclei;

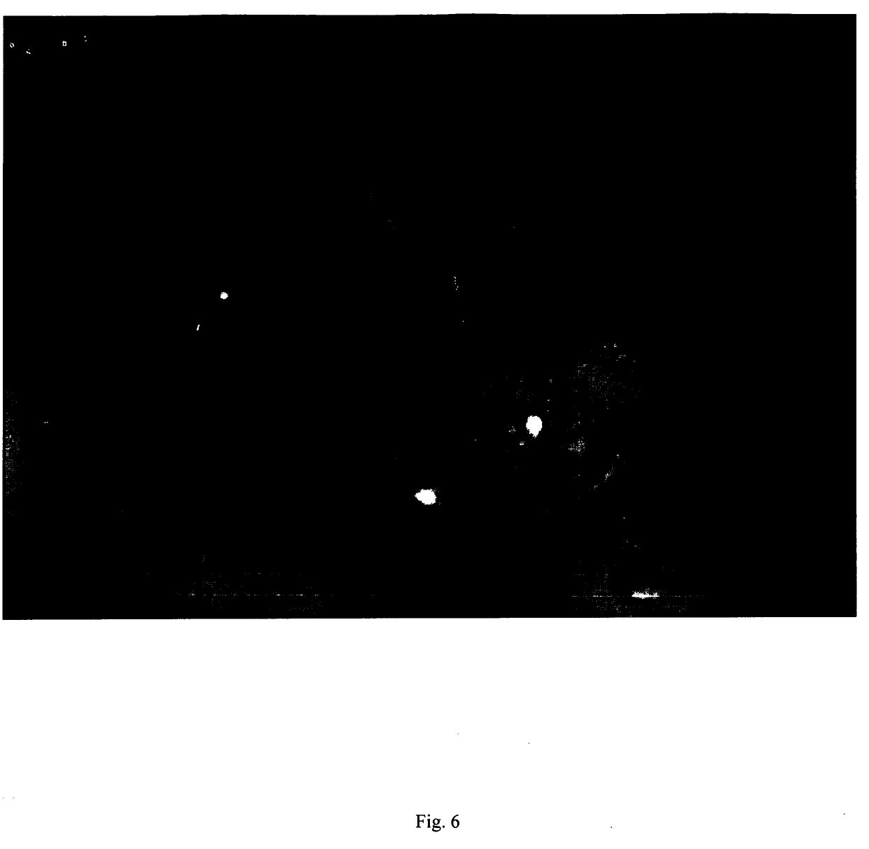

FIG. 6 is a fluorescence photograph of cells which have incorporated pure fluorescein as a negative control for the pictures represented in FIGS. 4 and 5;

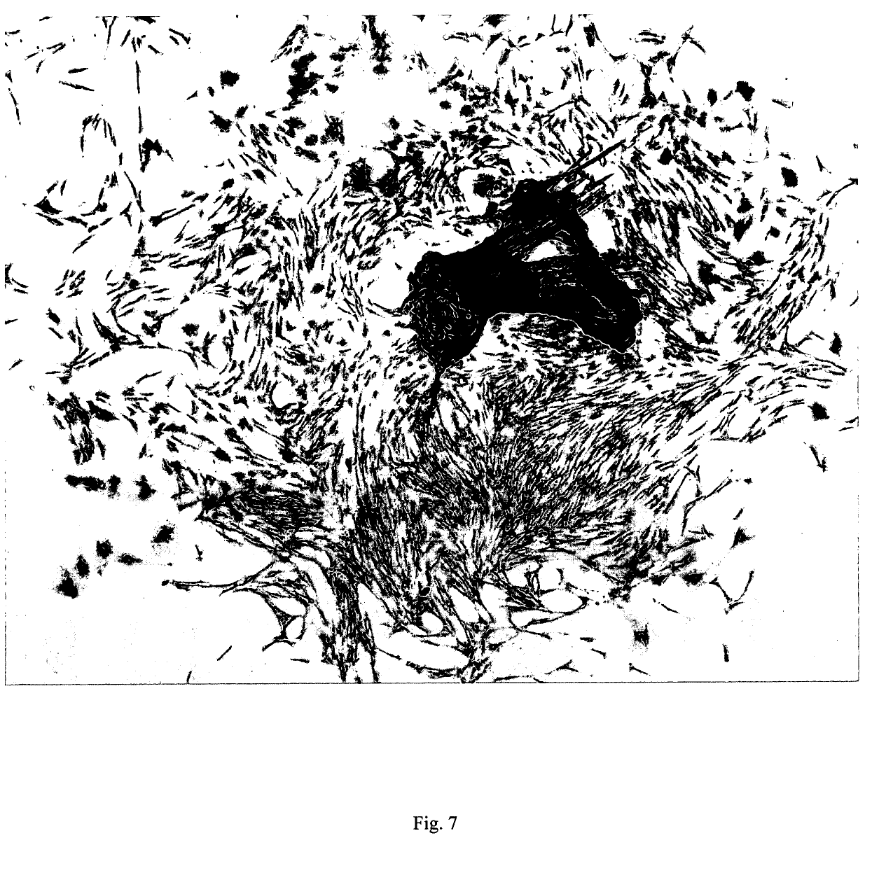

FIG. 7 is a light microscopic picture of a piece of skin treated with HMGA protein from which various skin cell types such as keratinocytes and fibroblasts grow out; and

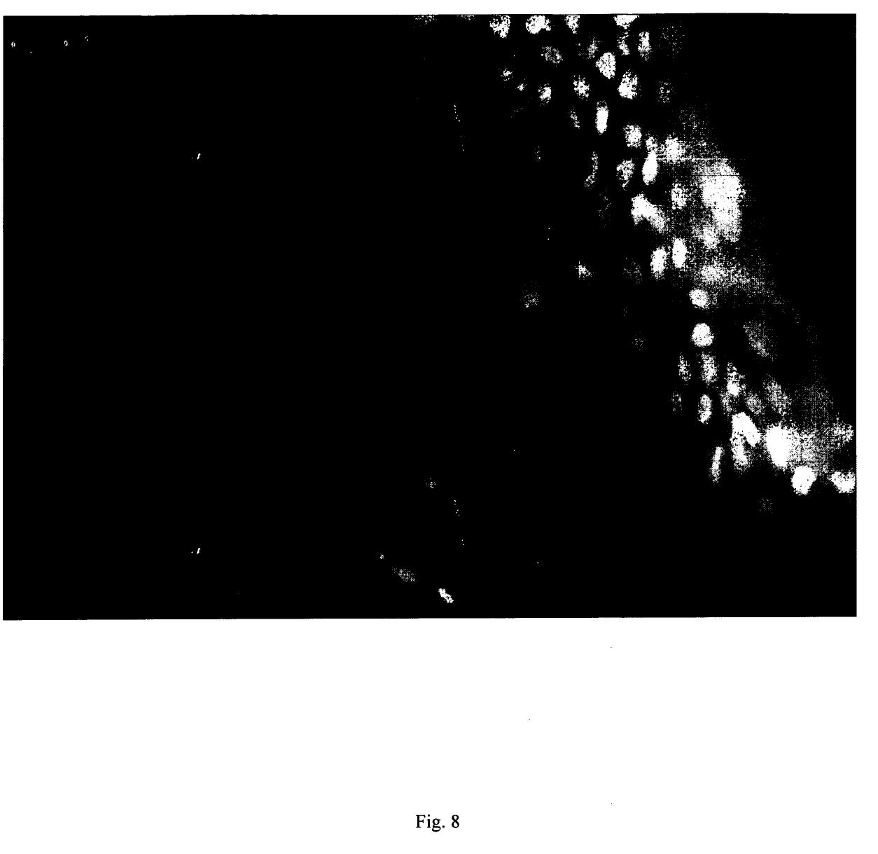

FIG. 8 is a light microscopic picture of a frozen section through the skin of rat in order to give evidence of the transfer of labelled HMGA1b protein after streptolysin O treatment.

### EXAMPLE 1

### Material and Methods

The following materials and methods were used in connection with the further examples, if not indicated to the contrary.

The transfer of proteins into eukaryotic cells using streptolysin O (SLO) was made by varying the Ca²⁺ concentration: in the absence of Ca²⁺ ions the cells are lysed, whereby subsequent addition of Ca²⁺ ions is suitable to close the cells again.

Cultivation of Cells

As a preparatory step the cells, in particular primary human fibroblasts, primary human chondrocytes, primary human keratinocytes, murine keratinocytes (MSC-P5, Cell Lines Service and Cellbank, Heidelberg) and human HeLa cells (ECACC N 85060701) were evenly distributed on a 6 well plate each containing 2.5 ml of medium 199 (Gibco-BRL; with 5% fetal calf serum) and incubated over night to a cell density of about 50-70% at 37° C. and 5% CO₂ aeration. As an alternative to the intact monolayer, prior to starting the experiments in some of the tests a cell-free space is created in the middle of the well by means of a scraper in order to simulate an artificial wound.

Method for Introducing HMGA1a into a Monolayer Cell Culture by Means of Streptolysin O

Prior to lysis the cells are washed twice with 1×PBS in order to remove medium residues containing Ca²⁺ ions. Lysis of the cells by means of streptolysin O (streptolysin O Reagent, Sigma-RBI) was done in Ca²⁺-free PBS buffer (Biochrom). The optimum streptolysin concentration at which most of the cells exhibit reversibly closing pores, was determined in preliminary experiments by means of trypan blue staining (Sigma-RBI) to be 0.1 U SLO/1 ml PBS for fibroblasts as well as for keratinocytes of the human skin. Per well 1 ml of the PBS/streptolysin mixture containing the desired concentration of the HMG protein to be tested (e.g. 100 ng/ml, 200 ng/ml and 1000 ng/ml HMGA1a) is added onto the fibroblasts, epithelial cells and keratinocytes, respectively. After incubation for 15 min at room temperatur 3 ml ice-cold medium 199 (with 5% fetal calf serum and 1.8 mM Ca²⁺) are added in order to close the cells again. The cells are incubated for 2 hours at 37° C. and 5% CO₂ aeration. Subsequently, the various reactions are replaced by 2.5 ml fresh medium and incubated as previously done until analysis is performed.

For the analysis, the cells are assessed under the microscope with regard to their morphology and colonisation of the cell-free space. Additionally, a methanol fixation of the cells with subsequent Giemsa staining (2% Giemsa solution) is performed in order to determine the number of mitotic events relative to the overall cell number.

Methods for Spontaneous Uptake of Fluorescence Labelled HMGA Proteins in Monolayer Cell Cultures

As a preparatory step the cells were incubated in Leighton tubes with inserted cover glasses (incubated over night at 37° C. and 5% CO₂ with 1 ml medium 199 each).

In order to make sure that no medium is left, the cells are washed twice with 1 ml PBS at room temperature. Subsequently, 200 μl serum-free medium and each 6 μg labelled HMGA protein and 6 μg fluorescein solution, as negative control, are added to the cells. The reaction is incubated for 1 h in the dark at 37° C. under 5% CO₂. After incubation 250 μl medium are added and the reaction is incubated for another 2 h 30 min. Subsequently, the cover glasses are briefly washed in PBS and covered on a microscopic slide. The analysis is performed after about 4 h.

Methods for Introducing Fluorescence Labelled HMGA1b in Monolayer Cell Cultures Using Streptolysin O

At first, HeLa cells and fibroblasts are incubated in Leighton tubes with each 1 ml medium 199 over night at 37° C. and 5% CO₂. The HeLa cell are cultivated in medium containing 20% fetal calf serum, and the fibroblasts in medium containing 5% calf serum.

In order to make sure that no medium is left, the cells are washed twice with PBS. The streptolysin O thawed at room temperature is diluted with PBS to a final concentration of 0.1 U/ml. Subsequently, 350 μl of the diluted streptolysin O and 6 μg labelled HMGA1b protein and 6 μg FLUOS solution, as a negative control, are added to HeLa cells and the fibroblasts. The reaction was incubated for 15 min at room temperature in the dark. Upon addition of 1 ml ice-cold medium 199 (with 20% fetal calf serum for the HeLa cells and 5% calf serum for the fibroblasts) the cells are closed again. After incubation for 1 h 30 min at 37° C. and 5% CO₂ the cover glasses are briefly washed in PBS and subsequently embedded on a microscope slide. The analysis is performed after about 2 h.

Methods for Introducing HMGA Proteins into Tissue Pieces of the Skin

The skin samples are obtained in a sterile manner and stored for the transport until the beginning of the experiment in Hank solution (Biochrom). The skin is cut into 0.5-1 mm pieces and is distributed among Sarstedt tubes for the experiment. The skin pieces are washed three times in 1×PBS by centrifugation for 5 min at 120×g and room temperature until all of the medium is washed away. After the last centrifugation step the supernatant is completely removed and the skin pieces are incubated for 15 min at room temperature in 1 ml SLO/PBS solution (0.1 U SLO/1 ml PBS) containing 1000 ng/ml of each of the HMG proteins (HMGA1a, HMGA1b, HMGA2) per reaction. In order to terminate lysis, 3 ml ice-cold medium 199 (with 20% fetal calf serum and 1.8 mM Ca²⁺) are added and the reactions are incubated for 2 hours at 37° C. and 5% CO₂ aeration. Subsequently, the skin pieces are distributed on sterile cover glasses, transferred in an inverted manner into humid chambers, each covered with 5 ml 20% medium 199 and further incubated at 37° C. and 5% CO₂ aeration. The pieces of skin are controlled under the microscope daily and the outgrowth of the various skin cell types documented for analysis purposes.

Methods for Introducing Fluorescence Labelled HMGA1b in Tissue Pieces of the Skin

Freshly prepared rat skin is cut into small pieces having a size of about 1-2 mm² in medium 199 containing 20% fetal calf serum using sterile instrumentation. The skin pieces are washed four times with PBS in order to remove all medium and Ca²⁺ remainings, respectively. Subsequently, the pieces of skin are transferred into an Eppendorf cup and 350 μl of the diluted streptolysin O (0.1 U/ml) and 6 μg labelled HMGA1b protein and 6 μg FLUOS solution, as negative control, are added to the pieces of skin. The reaction is incubated at room temperature for 15 min in the dark. The cells are closed again by the addition of 1 ml ice-cold medium 199 (with 20% fetal calf serum). After incubation for 1 h 30 min at 37° C. and 5% CO₂ frozen sections of the pieces of skin are prepared which are embedded in antifade.

The analysis is performed under fluorescence microscope after about 2-3 h.

Method for Fluorescence Labelling of HMG Proteins

The labelling is performed in accordance with the Fluorescein Labeling Kit of company Roche.

For each reaction 100 μg HMG protein, in particular HMGA1b protein, are lyophilised and resuspended in 100 μl PBS buffer. 1 μl FLUOS solution (2 mg/ml) is added to the dissolved HMGA1b and the reaction is incubated under stirring in the dark for 2 h at room temperature.

Sephadex-G-25 columns are equilibrated with 5 ml blocking solution and 30 ml PBS. Subsequently, the reaction is transferred onto the column and the labelled protein eluted with 3.5 ml PBS. The labelled protein is contained in the first two pools each consisting of 10 drops (about 0.5 ml).

The labelling is verified using HPLC (comparing the peaks of FLUOS solution/labelled HMGA, in particular HMGA1b and PA gel). The labelled protein could be observed to give a pronounced band under UV light as confirmed by Coomassie staining. About 60 μg/ml HMGA, in particular HMGA1b are labelled with fluorescein.

### EXAMPLE 2

### Cultivation of Endothelial Cells

Method:

For the cultivation of human endothelial cells artery preparations of the Arteria carotis were used. After removal, the tissue pieces were stored in Hank's solution until preparation. The preparation was performed in Hank's solution by carefully separating the Intima from the Media. Subsequently, tissue pieces having a size of about 1 mm were put onto cover glasses and cultivated in an inverted manner in the cell culture flask. Precultivation was made in 1 ml endothelial growth medium containing the respective growth factors, at 37° C. and 5% CO₂.

The cells were washed with PBS once they had reached a confluent cell density, trypsinised and split at 1:3.

Results:

After about 2 weeks first emigrated cells could be observed. The cells were subjected to immunohistochemistry using an anti-human CD 31 endothelial cell antibody for verification. It could be confirmed that the cells were human endothelial cells.

### EXAMPLE 3

### Proliferation Test of Endothelial Cells by the Application of HMGB1

Optimisation of the Cell Number

Method:

The proliferation test was performed using the Cell Proliferation ELISA, BrdU Kit of Roche.

For determining the optimum cell number, various dilutions of endothelial cells (10⁵-10² cells/100 μl) were prepared in 96 well plates and cultivated for 2 days at 37° C. and 5% CO₂ in the corresponding growth medium. The respective growth medium served as a control. Subsequently, 10 μl BrdU (concentration: 10 μM) were added to each well and the reaction incubated for 2 h 30 min at 37° C. and 5% CO₂. After the medium had been withdrawn, the cells were fixed by incubation for 30 min at room temperature using 200 μl/well fixation solution. Subsequently, 100 μl/well anti-BrdU antibody was added and incubated at room temperature for 90 min. In order to remove the non-bound antibody, the reaction was washed three times using 200 μl washing solution. In order to measure proliferation using calorimetric assays, 100 μl/well substrate were added to the reaction, stopped after about 5 min using 25 μl/well 1 M H₂SO₄ and absorption from 450 nm to 750 nm measured by means of an Anthos readers 2001. Three reactions were performed for each dilution.

Results:

After calculating the mean value of the parallel reactions, the various dilutions of the endothelial cells were represented as a diagram and analysed using Microsoft Excel. This resulted in an optimum cell number of 5,000-7,500 cells/100μl.

Administration of HMGB1 to Endothelial Cells

Method:

The cultivation was performed as described in example 2. After dispensing the optimum cell number of endothelial cells into the respective wells of a 96 well plate, the reaction was incubated for 24 h at 37° C. and 5% CO₂. Subsequently, the various HMGB1 concentrations (1 μg, 0.1 μg and 10 ng) were added. A total of three parallel approaches per HMGB1 concentration was performed. After cultivation for another 24 h, BrdU was added. The further steps were performed as described in example 3 under the heading “Optimisation of the cell number”.

Results:

A higher proliferation rate could be observed with endothelial cells to which HMGB1 had been administered, compared to the negative control. A correlation could be found between the proliferation rate and the concentration of administered HMGB1. In general, there was an increased rate of mitosis which could be detected microscopically, for cells to which HMGB1 had been administered.

### EXAMPLE 4

### Studying the Proangiogenic Effect of HMGB1 Using the Spheroid Model

Method:

As a preparatory step, human endothelial cells were prepared by cultivating them with the corresponding endothelial growth factor at 37° C. and 5% CO₂. The endothelial cells used for the experiments were taken from the 2^{nd} and 3^{rd} passage. After cultivation the cells were trypsinised and resuspended in the corresponding growth medium having a 20% content of methocel. After incubation for about 4 h the cells spontaneously formed three-dimensional cell spheres (spheroids), which were then embedded into a collagen gel. The following HMGB1 concentrations were added to the gel for a respective growth test: 2 μg/ml; 0.4 μg/ml and 0.08 μg/ml. The endothelial growth factor VEGF was used as a reference at a concentration of 25 ng/ml and, as a negative control, no growth factor was added to the reaction. After 2 weeks of incubation the reactions were analysed under an inverted microscope with a digital camera. The pictures were directly scanned into the picture analysis software analySIS of Soft Imaging System and analysed.

Results:

The additive sprout length, i.e. the length of the sprouts starting from the spheroid, could be analysed by means of the picture analysis software. A proangiogenic effect of HMGB1 could be observed at a concentration of 2 μg/ml. Compared to negative controls, an unambiguous formation of sprouts could be observed. Additionally, the combination of VEGF/HMGB1 showed a more pronounced formation of sprouts compared to HMGB1 only. The result is graphically depicted in FIG. 1.

### EXAMPLE 5

### Studies on the Proangiogenic Effect of HMGA1 in the Spheroid Model

Method:

In a preparatory step, human endothelial cells were cultivated in a corresponding endothelial growth medium at 37° C. and 5% CO₂. The endothelial cells used for the experiments were derived from the 2^{nd} and 3^{rd} passage. After cultivation the cells were trypsinised and resuspended in a respective growth medium having a 20% content of methocel. After incubation for about 4 h, the cells spontaneously formed three-dimensional cell spheres (spheroids), which were then embedded into a collagen gel. For a respective growth test a HMGA1 concentration of 2 μg/ml was added to this gel. Endothelial growth factor VEGF in a concentration of 25 ng/ml was used as a reference and, as a negative control, no growth factor was added to the reaction. After an incubation period of 2 weeks the reactions were analysed using an inverted microscope having a digital camera. The pictures were directly scanned into the picture analysis software analySIS of Soft Imaging System and analysed.

Results:

The cumulative length of sprouts, i.e. the overall length of sprout formation starting from the spheroid, could be determined by means of the picture analysis software. A proangiogenic effect of both HMGA1a as well as HMGA1b could be observed at a concentration of 2 μg/ml. Using a combination, HMGA1a showed an increased sprout formation compared to VEGF without HMG protein. The results are depicted as a diagram in FIG. 2.

### EXAMPLE 6

### Transfer of HMGA1a Proteins into Fibroblast Monolayer Cultures of Human Skin by Means of Streptolysin O

Compared to skin cells of the negative control (treatment with streptolysin O only), the cells treated with HMGA1a proteins showed a significant increase in proliferation rate. Corresponding to the increased proliferation rate, a significant increase in cell division rate could be detected when counting the number of cells undergoing mitosis in relation to the overall cell number, as also depicted in FIG. 3. Additionally, the cells treated with HMGA protein showed an increased motility which, for example, could be determined by the ingrowth of the cells into the cell-free area.

### EXAMPLE 7

### Transfer of Labelled HMGA1b Proteins into Cells Treated with SLO

Uptake of labelled HMGA1b proteins into the nucleus of HeLa cells (FIG. 2) as well as into the nucleus of fibroblasts (FIG. 5) could be shown after an incubation time of about 2 h. No positive signal could be observed when observing the cells immediately after the treatment with SLO, i.e. the uptake of the HMGA1b proteins into the nucleus lasts about 2 h. For example, for HeLa cells a ratio of HMGA1b positive cell nuclei compared to HMGA1b negative cell nuclei of 16:32 was determined.

The comparison to the negative control, i.e. the uptake of pure fluorescein, did not provide for a positive nucleus signal, but only a diffuse green staining of the cytoplasm as also depicted in FIG. 6.

### EXAMPLE 8

### Transfer of HMGA Proteins by Streptolysin O into Skin Samples of Man and Rat

Upon transfer of the HMGA proteins (HMGA1a, HMGA1b and HMGA2) into the cells of skin samples of man and rat, an increased proliferation could be observed with pieces of skin treated with HMGA proteins compared to the negative control. Different skin cell types (e.g. keratinocytes, fibroblasts) grew out of the analysed skin samples (see FIG. 7) which showed a high mitotic index and cell vitality despite treatment with streptolysin. Additionally, a significant increase in motility of the cells could be observed for skin samples treated with the HMGA proteins apart from an increased proliferation rate; which confirms that the therapeutic concept disclosed herein can be transferred from cell cultures to tissues.

### EXAMPLE 9

### Transfer of Labelled HMGA1b Proteins into the Skin of Rat by Means of Streptolysin O

The analysis of the frozen sections showed, as depicted in FIG. 8, that there is a strong nucleus positive signal related to HMGA1 for both the squamous epithelium as well as, in part, for the connective tissue. Again, as also observed for the cell culture, a nucleus-positive signal could only be observed after an incubation time of about 2 h. This confirms again that the nuclear transport of HMG proteins takes about 2 h.

Comparison with negative controls showed a diffuse green staining of the cytoplasm, whereby all cell nuclei were negatively stained.

### EXAMPLE 10

### Fluorescein Labelling of the HMGA1b Protein

The labelling was verified using HPLC (comparison of the peaks of FLUOS solution/labelled HMGA1b) and PA gel. The labelled protein gave a clear band under UV light which could be confirmed by coomassie staining. 60 μg/ml HMGA1b were labelled with fluorescein.

### EXAMPLE 11

### Expression Profile Analysis Using Microarrays for Determining the Mode of Action of HMGA1b Proteins in Connection with Tissue Regeneration

In order to analyse the molecular genetic mode of action of HMGA1b proteins in tissue regeneration, microarrays (Human 30K Array (A/B/C) of MWG-Biotech) were used in order to analyse the expression pattern of the skin samples treated with HMGA1 b protein compared to untreated skin samples.

For such purpose, a human skin sample was cut into 0.5-1 mm pieces and distributed among Sarstedt tubes. The pieces of skin were washed three times in 1×PBS by centrifugation for 5 min with 120×g at room temperature until all of the medium was washed away. After the last centrifugation step the supernatant was completely removed and the pieces of skin incubated for 15 min at room temperature in 1 ml SLO/PBS solution (0.1 U SLO/1 ml PBS) with 1000 ng/ml of the HMGA1b protein per reaction and without the protein, respectively, as a negative control. In order to terminate lysis, 3 ml ice-cold medium 199 (containing 20% fetal calf serum and 1.8 mM Ca²⁺) were added to each reaction and the reactions were incubated for 12 hours at 37° C. and 5% CO₂ aeration.

The isolation of the RNA from the skin samples was performed using the RNeasy RNA Isolation Kit (Qiagen), in accordance with the protocol “Isolation of total RNA from Heart, Muscle, and Skin Tissue” of the manufacturer and an additional DNase digestion for 2×15 min at 25° C. The synthesis of the ss cDNA was performed in accordance with the standard protocol for Superscript (Invitrogen). The fluorescence labelling of the cDNA was performed using Cy3-UTP and Cy5-UTP by means of “direct-labelling” of the ss cDNA.

For hybridisation, the labelled cDNA was denatured for 3 min at 95° C., incubated on ice for 3 min and optionally precipitating precipitates dissolved at 42° C. The hybridisation was performed in accordance with the instructions of the manufacturer of the microarray (MWG Biotech), using Microarray Gene Frames at 42° C. for 16-24 h. The following washing steps in 2×SSC, 0.1% SDS (wash buffer 1), 1×SSC (wash buffer 2) and 0.5×SSC (wash buffer 3) were performed in accordance with the instructions of the manufacturer. The analysis was performed using an Affymetrix 428 Array Scanner.

Important information could be obtained on the mode of action of the HMGA proteins and the HMGA1b protein, respectively, in the proliferation and reactivation of skin cells by analysing the expression pattern. The result of the analysis showed the effect of the addition of the HMGA1b protein on gene expression of the target tissue by means of interaction of the HMGA1b protein with its partners at the protein-DNA as well as protein-protein level. The expression pattern of a variety of genes which are involved in wound healing as well as regeneration of the skin is regulated by this mechanism of interaction of the protein. The important role of the HMGA protein and the HMGA1b protein, respectively, in the tissue regeneration of the skin as well as for wound healing and anti-aging, i.e. for tissue rejuvenation, could be verified by detecting the re-expression of fetal genes in adult tissue.

### EXAMPLE 12

### Expression Profile Analysis by Means of Microarrays for Determining the Mode of Action of HMGA Proteins on the Repair of DNA Damages

Arrays were used for analysing the molecular genetic mode of action of the HMGA proteins in connection with the repair of DNA damages (Atlas-Arrays 1.2 of Clontech, # 7850-1, comprising 1176 gene sequences of the human genome) in order to analyse the expression pattern of keratinocytes treated with HMGA proteins compared to untreated keratinocytes.

The isolation of the RNA from the cells was performed using the RNeasy RNA Isolation Kit (Qiagen) following the protocol “Isolation of total RNA from Heart, Muscle, and Skin Tissue” of the manufacturer and an additional DNase digestion for 2×15 min at 25° C. The synthesis of the ss cDNA was performed according to the standard protocol for Superscript (Invitrogen), the cDNA radioactively labelled (³²P) and then used for hybridisation.

The proteins HMGA1a, HMGA1b and HMGA2 were each used in an amount of 6 μg. It could be shown that the administration of recombinant, human HMGA protein up-regulated genes the protein of which is related to the repair of DNA damages. Samples are shown in Tab. 3. ATM is a protein kinase which is activated upon double-strand breaks. ATM phosphorylates further key proteins in response to double-strand breaks (Yosef Shiloh: ATM and related proteins kinases: safeguarding genome integrity. Nature Review Cancer 2003: 3, 155-168). TOP1 is topoisomerase 1 which is involved in DNA repair processes (Pastor N, Cortes F: DNA topoisomerase activities in Chinese hamster radiosensitive mutants after X-ray treatment. Cell Biol In7 2002:26, 547-555).

TABLE 3
Up-regulation of genes the proteins of which are related to the repair of
DNA damages, as a consequence of the administration of HMGA proteins.
The average value (three reactions) of the quotient of the expression in the
treated reaction and the expression in the untreated reaction is indicated

| | HMGA protein | | |
|---|---|---|---|
| gene | HMGA1a | HMGA1b | HMGA2 |
| ATM | 1.43 | 2.16 | 2.25 |
| TOP1 | 4.37 | 3.89 | 4.53 |

### EXAMPLE 13

### Spontaneous Transfer of Fluorescence Labelled HMGA1b and HMGA2 Proteins into Human Epithelial Cells

The spontaneous uptake of labelled HMGA proteins into the nucleus of the cells, in particular of HMGA1b and HMGA2, could be shown after an incubation time of about 4 h. If viewed immediately after the treatment, the cells did not show any positive signal in the cell nuclei, i.e. the uptake of the HMGA proteins into the cell nucleus took lasted about 4 h.

Compared to the negative control, i.e. the uptake of pure fluorescein, there was no nucleus positive signal but only a green diffuse staining of the cytoplasm. Depending on the individual experiment and as generally described in example 1, 50-100% of the cells showed nuclear fluorescence staining. As this staining is only visible at comparatively high protein concentrations, it can be assumed that all cells show take up HMGA proteins.

By analysing the obtained expression pattern important information could be gained on the mode of action of HMGA proteins and the HMGA1b protein, respectively, on proliferation and re-activation of skin cells. The result of this analysis showed the impact of the addition of HMGA1b protein on the gene expression of the target tissue by means of interaction of the HMGA1b protein with its partners at the protein-DNA as well as protein-protein level. The expression pattern of a variety of genes which are involved in wound healing and regeneration of the skin, is controlled by this mechanism of interaction of the protein. The detection of the re-expression of fetal genes in adult tissue verified the important role of the HMGA protein and the HMGA1b protein, respectively, in the tissue regeneration of the skin as well as in wound healing and anti-aging, i.e. for tissue rejuvenation.

### EXAMPLE 14

### Immunohistochemistry with an anti-HMGB1 Antibody

All immunohistochemical studies on paraffin sections (5 μm) of human tissue and tissue samples of the dog were carried out using a polyclonal antibody from goat (sc-12523, Santa Cruz Biotechnology, Santa Cruz, USA) which is directed against a peptide of the internal region of the human HMGB1 protein. The antibody used detected HMGB1 and, to a lesser extent, the HMGB2 protein.

### EXAMPLE 15

### Immunohistochemical Detection of HMGB1 at Patches of Skin Afflicted by Psoriasis and Comparison with Non-Afflicted Areas of the Patient

The surprising result of immunohistochemical studies on skin areas afflicted by psoriasis in comparison to non-afflicted areas of patients (three tissue pairs), was that the HMGB1 protein is significantly higher expressed in the capillary of the afflicted areas compared to control tissues. A positive signal was primarily found in the cytoplasm of afflicted endothelial cells and was particularly pronounced in those areas of the capillaries where there was proliferation activity in psoriasis. In ⅔ tissue pairs monocytes present in the psoriatic areas were also strongly stained positive in the cytoplasm.

Insofar, the use of inhibitors of HMGB1 protein as disclosed herein, is a suitable means for the treatment of this disease.

### EXAMPLE 16

### Immunohistochemical Detection of HMGB1 Protein in Malignant Histiocytosis of the Dog

Malignant histiocytosis is a comparatively rare disease of the dog having bad prognosis. An increased occurrence can be observed with the Berner Sennenhund. The disease is, among others, characterised by the proliferatin of macrophages. Here, tissue samples from five dogs were examined. A strong immune reaction of the macrophages was found in all samples examined and was significantly more pronounced than in control tissue. The protein was predominantly found in the cytoplasm of macrophages.

Because of this, the use of the inhibitors against HMGB1 protein as disclosed herein is a suitable means for the treatment of this disease.

### EXAMPLE 17

### Immunohistochemical Detection of HMGB1 Protein in Chronic Superficial Keratitis of the Cornea of Dogs

Chronic superficial keratitis of the cornea of dogs is a change in tissue which involves inflammatory processes and neoangiogenesis. As a result of this disease the respective animals might get blind. Cytological preparations of diseased animals were prepared and analysed using immunohistochemistry. A significant positive signal for HMGB1 was obtained for the lymphocytes.

Because of this, the use of inhibitors of HMGB1 protein, as disclosed herein, is a suitable means for the treatment of this disease.

### EXAMPLE 18

### Regulation of VEGF1 in endothelial cells by HMGB1

The preparation and cultivation of human endothelial cells was performed as described in example 2. After addition of human recombinant HMGB1 (rHMGB1) using the following concentrations: 80 ng/ml, 200 ng/ml and 400 ng/ml, the cells were harvested 10 hours after the addition and RNA isolation was performed (for RNA preparation, Northern blot hybridisation, see Flohr et al., Anticancer Res. 2001; 21: 3881-3886). The quantification was performed by means of Northern blot analysis using a cDNA sequence of the open reading frame of human VEGF A. Compared to controls which did not contain any protein, there was a concentration-dependent increase of VEGF A expression by a factor of 1.6, 2.8 and 3.2 (average values from two determinations).

Because of this, the use of inhibitors of HMGB1 proteins as disclosed herein, is a suitable means for reducing the VEGF A mediated angiogenesis and thus an appropriate means for the treatment of tumors. In contrast thereto, HMGB1 and a nucleic acid coding therefor may be administered as an effector for increased expression of VEGF A and may thus be used as agents for the treatment of diseases where VEGF A is administered.

### EXAMPLE 19

### Proliferation and migration of keratinocytes by HMGB1

The preparation and cultivation of human skin explants in vitro was performed as described in example 1. Four parallel cultures of two donors were initiated. There were five explants per culture reaction. Human recombinant HMGB1 (rHMGB1) was added at a concentration of 200 ng/ml. The determination of the cell number by microscopy was performed 8 days after starting the cultivation by counting the keratinocytes which had grown out from the explants (morphological differentiation of keratinocytes vs. fibroblasts). Compared to the control there was an average increase in the number of keratinocytes by 28%.

This example confirms the usefulness of HMGB1 for the proliferation and migration of keratinocytes.

The features of the invention disclosed in the preceding specification, the claims, the figures as well as the sequence protocol which is part of the description, may be used individually or in any combination for the practising of the invention in its diverse embodiments.

1. Use, particularly in vitro use, of one or several nucleic acid(s), the transcription product(s) thereof and/or the translation product(s) thereof in a process, whereby the process is selected from the group comprising angiogenesis, neovascularization, transmyocardial revascularization, wound healing, angiogenesis following wounding, epithelialization and healing of tooth and bone implants,
whereby the nucleic acid(s) is/are one(s) that code(s) for HMGB1 or a part thereof.

2. Use of one or several nucleic acid(s), the transcription product(s) thereof and/or the translation product(s) thereof for the manufacture of a medicament for the prevention and/or treatment of a disease, whereby the disease is selected from the group which is related to lacking or excessive angiogenesis or neovascularization or wound healing, or requires transmyocardial revascularization,
whereby the nucleic acid is one that codes for HMGB1 or a part thereof.

3. Use, particularly in vitro use, of one or several nucleic acid(s), the transcription product(s) thereof and/or the translation product(s) thereof for a process, whereby the process is selected from the group comprising angiogenesis, neovascularization, transmyocardial revascularization, wound healing, angiogenesis following wounding, epithelialization and healing of tooth and bone implants,
whereby the nucleic acid(s) is/are selected from the group comprising genes for the high mobility group proteins.

4. Use of one or several nucleic acid(s), the transcription product(s) thereof and/or the translation product(s) thereof for the manufacture of a medicament for the prevention and/or treatment of a disease, whereby the disease is selected from the group which is related to lacking or excessive angiogenesis or neovascularization or wound healing, or requires transmyocardial revascularization,
whereby the nucleic acid(s) is/are selected from the group comprising the genes for high mobility group proteins.

5. Use of one or several nucleic acid(s), the transcription product(s) thereof and/or the translation product(s) thereof for the manufacture of a medicament for the prevention and/or treatment of a disease, particularly of claim 2 and/or claim 4, characterised in that the disease is selected from the group comprising diabetic retinopathy, proliferative retinopathia diabetica, diabetic nephropathy, macular degeneration, arthritis, endometriosis, pannus, histiocytosis, psoriasis, rosacea, small varicose veins, eruptive hemangioma, tumor diseases, cavernoma, lip angioma, haemangiosarcoma, haemorrhoids, artherosclerosis, angina pectoris, ischemia, infarction, basalioma, squamous carcinoma, melanoma, Kaposi's sarcoma, tumors, gestosis, infertility, acute traumatic wounds, thermal wounds, chemical wounds, surgical wounds and chronic wounds.

6. Use according to claim 5, characterised in that the chronic wound is selected from the group comprising decubitus, ulcus cruris, ulcus cruris venosum, ulcus cruris arteriosum, diabetic ulcus, decubital ulcer, chronic post-traumatic wound and diabetic foot ulcers.

7. Use according to any of claims 3 to 6, characterised in that the high mobility group protein is selected from the group comprising the HMGA family, the HMGB family and the HMGN family.

8. Use according to any of claims 3 to 7, characterised in that the high mobility group protein is selected from the HMGB family.

9. Use according to claim 8, characterised in that the high mobility group protein is selected from the group comprising HMGB1, HMGB2 and HMGB3.

10. Use according to claim 9, characterised in that the high mobility group protein is HMGB1.

11. Use according to any of claims 3 to 7, characterised in that the high mobility group protein is selected from the HMGA family.

12. Use according to claim 11, characterised in that the high mobility group protein is selected from the group comprising HMGA1a, HMGA1b, HMGA1c and HMGA2.

13. Use according to claim 12, characterised in that the high mobility group protein is HMGA1a.

14. Use according to any of the preceding claims, characterised in that one high mobility group protein is selected from the HMGA family, and a second high mobility group protein is selected from the HMGB family, whereby the protein of the HMGA family is preferably HMGA1a and the protein of the HMGB family is preferably HMGB1.

15. Use according to any of the preceding claims, characterised in that additionally VEGF and/or a nucleic acid coding therefor, is used.

16. A method for affecting angiogenesis or neovascularization or wound healing of a tissue comprising the following steps:
a) providing a tissue or a part thereof,
b) adding one or several nucleic acid(s), transcription product(s) thereof and/or translation product(s) and
c) incubating the tissue with the nucleic acid(s), the transcription product(s) thereof and/or the translation product(s) thereof,
whereby the nucleic acid(s) is/are selected from the group comprising the genes for the high mobility group proteins, and, optionally,
d) obtaining or recovering the tissue or an intermediate thereof.

17. The method according to claim 16, characterised in that the tissue or a part thereof is incubated with VEGF and/or a nucleic acid coding therefor.

18. The method according to claims 16 or 17, characterised in that the method is an in vitro method.

19. The method according to any of claims 16 to 18, characterised in that the tissue is an explanted tissue or an in vitro cultured tissue.

20. The method according to any of claims 16 to 19, characterised in that the nucleic acid(s), the transcription product(s) thereof and the translation product(s) thereof is/are such as described in any of the preceding claims.

21. The method according to any of claims 16 to 20, characterised in that two or more of the HMGB proteins or of the nucleic acid(s) coding therefor are used, whereby preferably one high mobility group protein is selected from the HMGA family and a second high mobility group protein is selected from the HMGB family, whereby the protein of the HMGA family is preferably HMGA1a, and the protein from the HMGB family is preferably HMGB1.

22. Use according to any of the preceding claims, characterised in that in addition to the nucleic acid(s), the transcription product(s) thereof and/or the translation product(s) thereof, whereby the nucleic acid is selected from the group comprising the genes for the high mobility group protein, a nucleic acid, the transcription product thereof or the translation product thereof, is used, whereby the nucleic acid is selected from the group comprising the gene for vascular endothelial growth factor.

23. A pharmaceutical formulation comprising one or several nucleic acid(s), the transcription product(s) thereof and/or the translation product(s) thereof, as described in any of the preceding claims, and a pharmaceutically acceptable carrier.

24. A carrier material comprising one or several nucleic acid(s), the transcription product(s) thereof and/or the translation product(s) thereof, whereby the nucleic acid(s), the transcription product(s) thereof and/or the translation product(s) thereof is/are such as described in any of the preceding claims.

25. The carrier material according to claim 24, characterised in that the carrier material consists of a material which is selected from the group comprising cellulose, agarose, collagen, silicone, silicon, plastics, gels, hydrogels, matrices based on fibrin, man-made continuous filament yarn, hydrocolloids, lipocolloids, polyurethane, polyurethane resins, plaster, synthetic biomaterials, thermoplastic plastics, zinc glue, polyester foam, polyisobutylene, buffer, stabilizers, bacteriostatics and moisturizer.

26. The carrier material according to claims 24 or 25, characterised in that the carrier material is serving as an implant or for wound healing.

27. A wound cover material comprising a basic cover material and one or several nucleic acid(s), the transcription product(s) thereof and/or the translation product(s) thereof, whereby the nucleic acid(s), the transcription product(s) thereof and/or the translation product(s) thereof is/are such as described in any of the preceding claims.

28. The wound cover material according to claim 27, characterised in that the cover material is selected from the group comprising hydrocolloidal dressings, calcium alginate dressings, compresses and overlays of activated carbon, overlays of foamed plastic, film dressings, transparent dressings, silicone foam dressings, fleece overlays, hydrocellular dressings, hydroselective wound overlays, absorbing wound pads, spray dressings, gauze of man-made continuous filaments, cotton gauze, paraffin gauze, silver coated wound dressings and hydropolymer/foam dressings.

29. A formulation comprising one or several nucleic acid(s), the transcription product(s) thereof and/or the translation product(s) thereof, whereby the nucleic acid(s), the transcription product(s) thereof and/or the translation product(s) thereof is/are such as described in any of the preceding claims, and a carrier phase, whereby the carrier phase is preferably selected from the group comprising creams, fatty ointments, emulsions (oil in water (O/W); water in oil (W/O); water in oil in water (W/O/W)); microemulsions, modified emulsions, nanoparticles/nanoemulsions, liposomes, hydrodispersion gels (hydrogels, alcoholic gels, lipogels, tenside gels), gel-creams, lotions, oils/oil baths and sprays.

30. A method for the screening of a compound for promoting and/or inhibiting a process, whereby the process is selected from the group comprising angiogenesis, neovascularization, transmyocardial revascularization and wound healing, comprising the following steps:
a) providing a test system for the process;
b) providing a candidate compound; and
c) testing the candidate compound and determining the reaction caused by the candidate compound in the test system.

31. A method for the screening of a compound for promoting and/or inhibiting a process, whereby the process is selected from the group comprising angiogenesis, neovascularization, transmyocardial revascularization and wound healing, comprising the following steps:
a) providing a test system for the process;
b) providing a reference compound;
c) testing the reference compound in the test system and determining the reaction caused by the reference compound in the test system;
d) providing a candidate compound;
e) testing the candidate compound in the test system and determining the reaction caused by the candidate compound in the test system; and
f) comparing the reaction of the reference compound in the test system to the reaction of the candidate compound in the test system.

32. A method for the screening of a compound for the promotion and/or inhibition of a process, whereby the process is selected from the group comprising angiogenesis, neovascularization, transmyocardial vascularization and wound healing, comprising the following steps:
a) providing a test system for the process;
b) providing a reference compound, whereby the reference compound has a marker;
c) testing the reference compound in the test system and determining the reaction caused by the reference compound in the test system;
d) providing the candidate compound; and
e) testing the candidate compound in the test system, whereby the test system comprises the reference compound, and determining the reaction of the test system, whereby the amount of released reference compound and/or released marker of the reference compound is determined.

33. A method for the screening of a compound for the promotion and/or inhibition of a process, whereby the process is selected from the group comprising angiogenesis, neovascularization, transmyocardial vascularization and wound healing, comprising the following steps:
a) providing a test system for the process;
b) providing a candidate compound, whereby the candidate compound has a marker;
c) testing the candidate compound in the test system and determining the reaction caused by the candidate compound in the test system;
d) providing a reference compound; and
e) testing the reference compound in a test system, whereby the test system comprises a candidate compound, and determining the reaction of the test system, whereby the amount of released candidate compound and/or of released marker of the candidate compound is determined.

34. The method according to any of claims 30 to 33, characterised in that the test system is an in vitro test system or a in vivo test system.

35. The method according to any of claims 30 to 34, characterised in that the reaction of the reference compound and/or of the candidate compound is a promotion of the process, and whereby preferably the candidate compound is a compound for promoting the process if the reaction of the candidate compound in the test system is identical or more pronounced than the reaction of the reference compound.

36. The method according to any of claims 30 to 34, characterised in that the reaction of the reference compound and/or the candidate compound is an inhibition of the process, and whereby preferably the candidate compound is a compound for inhibiting the process, if the reaction of the test system caused by the candidate compound is a reaction which is less pronounced than the one caused by the reference compound in the test system.

37. The method according to any of claims 30 to 36, characterised in that the reference compound comprises one or several nucleic acid(s), the transcription product(s) thereof and/or the translation product(s) thereof, whereby the nucleic acid is selected from the group comprising genes for high mobility group proteins, preferably as defined in any of the preceding claims.

38. The method according to any of claims 30 to 36, whereby the process is the inhibition of angiogenesis.

39. Use of a method according to any of claims 30 to 38 for the screening of a compound for the treatment and/or prevention of a disease, whereby the test system provided is a test system for the respective disease.

40. Use according to claim 39, characterised in that the disease is selected from the group comprising diseases which require the promotion or inhibition of angiogenesis or neovascularization, or transmyocardial revascularization or wound healing.

41. Use according to claim 40, characterised in that the disease is selected from the group comprising diabetic retinopathy, proliferative retinopathia diabetica, diabetic nephropathy, macular degeneration, arthritis, endometriosis, pannus, histiocytosis, psoriasis, rosacea, small varicose veins, eruptive hemangioma, tumor diseases, cavemoma, lip angioma, haemangiosarcoma, haemorrhoids, artherosclerosis, angina pectoris, ischemia, infarction, basalioma, squamous carcinoma, melanoma, Kaposi's sarcoma, tumors, gestosis, infertility, acute traumatic wounds, thermal wounds, chemical wounds, surgical wounds and chronic wounds.

42. Use according to any of claims 39 to 41, characterised in that the disease is a tumor disease, whereby preferably the tumor diseases comprise necrotic cells, preferably necrotic tumor cells.

43. Compound obtainable by a method according to any of claims 30 to 38.

44. Use of a compound according to claim 43 for the manufacture of a medicament, preferably for the treatment and/or inhibition of a disease, as defined in any of the preceding claims.

45. Use, particularly in vitro, of a nucleic acid, the transcription product thereof and/or the translation product thereof, for a process, whereby the process is selected from the group comprising tissue regeneration, repair of DNA damages, wound healing, cell mobility, angiogenesis in the wound area, epithelialization, tissue aging, prevention of tissue aging, rejuvenation of tissue, vascularization after cardiac infarction and healing of tooth and bone implants,
whereby the nucleic acid is selected from the group comprising genes for basic DNA binding proteins.

46. Use, particularly in vitro use, of a nucleic acid, the transcription product thereof and/or the translation product thereof, for a process, whereby the process is selected from the group comprising dedifferentiation of cells and re-programming of cells, for tissue build-up and/or tissue regeneration, in particular based on dedifferentiation and/or differentiation of the tissue to be build up or to be regenerated,
whereby the nucleic acid is selected from the group comprising genes for basic DNA binding proteins.

47. Use of a nucleic acid, the transcription product thereof and/or the translation product thereof, for the manufacture of a medicament for prevention and/or treatment of a disease, whereby the disease is selected from the group comprising diseases which require the repair DNA damages, diseases which require tissue regeneration, diseases which require wound healing, diseases which go along with tissue aging, diseases which require tooth and bone implants, diseases which go along with tissue aging, wound healing disorders, skin diseases, xeroderma pigmentosum, leather skin, skin cancer, skin cancer after burn, skin aging after burn, burn and cardiac infarction,
whereby the nucleic acid is selected from the group comprising genes for basic DNA-binding proteins.

48. Use of a nucleic acid, the transcription product thereof and/or the translation product thereof, for the manufacture of a cosmetic product, preferably a cosmetic product for tissue regeneration, wound healing, prevention of leather skin, prevention of skin cancer, in particular skin cancer after sun burn, skin aging, in particular skin aging after sun burn, tissue aging inhibition and/or tissue juvenation,
whereby the nucleic acid is selected from the group comprising genes for basic DNA-proteins.

49. Use of a nucleic acid, the transcription product thereof and/or the translation product thereof for the manufacture of a medicament for the prevention and/or treatment of a disease, whereby the disease is selected from the group comprising skin diseases, xeroderma pigmentosum, leather skin, skin cancer, skin cancer after sun burn, sun burn, acute wounds and chronic wounds,
whereby the nucleic acid is selected from the group comprising genes for basic DNA-binding proteins.

50. Use according to claim 49, characterised in that the acute wound is selected from the group comprising acute traumatic wounds, thermal wounds, chemical wounds and surgical wounds.

51. Use according to claim 49, characterised in that the chronic wound is selected from the group comprising decubitus, ulcus cruris, ulcus cruris venosum, ulcus cruris arteriosum, diabetic ulcus, decubital ulcer, chronic post-traumatic wounds and diabetic foot ulcer.

52. Use according to any of claims 45 to 51, characterised in that the basic DNA-binding protein is selected from the group comprising high mobility group proteins.

53. Use according to any of claims 45 to 52, characterised in that the high mobility group protein is selected from the group comprising HMGA, HMGB and HMGN.

54. Use according to any of claims 45 to 53, characterised in that the high mobility group protein is a protein of the HMGA family.

55. Use according to claim 54, characterised in that the protein is selected from the group comprising HMGA1a, HMGA1b and HMGA2.

56. Use according to any of claims 45 to 55, characterised in that the nucleic acid is selected from the group comprising nucleic acids according to SEQ. ID. NO. 31 to SEQ. ID. NO. 64 and respective derivatives.

57. Use according to any of claims 45 to 56, characterised in that the translation product is selected from the group comprising polypeptides having a sequence according to SEQ. ID. NO. 1 to SEQ. ID. NO. 30 and the respective derivatives.

58. Use according to claim 57, characterised in that the protein comprises a modification, whereby the modification is selected from the group comprising phosphorylation and acetylation.

59. A method for the regeneration of tissue comprising the following steps:
a) providing a tissue or a part thereof,
b) adding a nucleic acid, the transcription product thereof and/or the translation product thereof; and
c) incubating the tissue and the nucleic acid, the transcription product thereof and/or the translation product thereof,
whereby the nucleic acid is selected from the group comprising genes for basic DNA-binding proteins, and, optionally,
d) obtaining or recovering the regenerated tissue or a intermediate form thereof.

60. The method according to claim 59, characterised in that the method is an in vitro method.

61. The method according to claim 59 or 60, characterised in that the tissue to be regenerated is different or identical to the tissue provided in step a).

62. The method according to any of claims 59 to 61, characterised in that the tissue to be regenerated and/or the tissue provided in step a) is/are independently selected from each other from the group comprising skin tissue, fatty tissue, cartilage tissue, muscle tissue, cells of the blood and of the haemogram and nerve cells.

63. The method according to any of claims 59 to 62, characterised in that the nucleic acid, the transcription product and/or the translation product is/are such as described in any of the preceding claims.

64. A method for the dedifferentiation and/or reprogramming of cells comprising the following steps:
a) providing one or several cells,
b) adding a nucleic acid, the transcription product thereof and/or the translation product thereof, and
c) incubating the cell and the nucleic acid, the transcription product thereof and/or the translation product thereof,
whereby the nucleic acid is selected from the group comprising genes for basic DNA-binding proteins.

65. The method according to claim 64, characterised in that the method is an in vitro method.

66. The method according to claim 64 or 65, characterised in that the method further comprises the following step:
d) obtaining a dedifferentiated and/or reprogrammed cell.

67. The method according to any of claims 64 to 66, characterised in that the dedifferentiated cell(s) and/or the reprogrammed cell(s) and/or the cell(s) provided according to step a) is/are independently selected from the group comprising cells of the epidermis, cells of the skin, cells of the fatty tissue, cells of the cartilage tissue, cells of the muscle tissue, cells of the blood, cells of the blood-forming tissues and nerve cells.

68. The method according to any of claims 64 to 67, characterised in that the nucleic acid, the transcription product thereof and/or the translation product thereof is as defined in any of the preceding claims.

69. Pharmaceutical formulation comprising a nucleic acid, a transcription product thereof and/or translation product thereof, as defined in any of the preceding claims, and a pharmaceutically suitable carrier.

70. A carrier material comprising a nucleic acid, the transcription product thereof and/or the translation product thereof, whereby the nucleic acid, the transcription product thereof and/or the translation product thereof is as defined in any of the preceding claims.

71. The carrier material according to claim 70, characterised in that the carrier material comprises a material selected from the group comprising cellulose, agarose, collagen, silicone, silicon, plastics, gels, hydrogels, matrices based on fibrin, man-made continuous filament yarn, hydrocolloids, lipocolloids, polyurethane, polyurethane resins, plaster, synthetic biomaterials, thermoplastic plastics, zinc glue, polyester foam, polyisobutylene, buffer, stabilizers, bacteriostatics and moisturizers.

72. The carrier material according to claims 70 or 71, characterised in that the carrier material is serving as an implant or for wound healing.

73. A wound covering material comprising a basic cover material and a nucleic acid, the transcription product thereof and/or the translation product thereof, whereby the nucleic acid, the transcription product thereof and/or the translation product thereof is/are as defined in any of the preceding claims.

74. The wound covering material according to claim 73, characterised in that the cover material is selected from the group comprising hydrocolloidal dressings, calcium alginate dressings, compresses and overlays of activated carbon, overlays of foamed plastic, film dressings, transparent dressings, silicone foam dressings, fleece overlays, hydrocellular dressings, hydroselective wound overlays, absorbing wound pads, spray dressings, gauze of man-made continuous filaments, cotton gauze, paraffin gauze, silver coated wound dressings and hydropolymer/foam dressings.

75. A cosmetic formulation comprising a nucleic acid, the transcription product thereof and/or the translation product thereof, whereby the nucleic acid, the transcription product thereof and/or the translation product thereof is as described in any of the preceding claims, and a carrier phase, whereby the carrier phase is preferably selected from the group comprising creams, fatty ointment, emulsions (oil in water (O/W); water in oil (W/O); water in oil in water (W/O/W)); microemulsions, modified emulsions, nanoparticles/nanoemulsions, liposomes, hydrodispersion gels (hydrogels, alcohol gels, lipogels, tenside gels), gel-creams, lotions, oils/oil baths and sprays.

76. A method for the screening of a compound for promoting and/or inhibiting a process, whereby the process is selected from the group comprising tissue regeneration, repair of DNA damages, wound healing, cell mobility, angiogenesis in the wound area, epithelialization, tissue aging, inhibition of tissue aging, tissue rejuvenation, vascularization after cardial infarction and healing of tooth and bone implants, comprising the following steps:
a) providing a test system for the process;
b) providing a candidate compound; and
c) testing the candidate compound and determining the reaction caused by the candidate compound in the test system.

77. A method for the screening of a compound for promoting and/or inhibiting a process, whereby the process is selected from the group comprising tissue regeneration, a repair of DNA damages, wound healing, cell mobility, angiogenesis in the wound area, epithelialization, tissue aging, inhibition of tissue aging, tissue rejuvenation, vascularization healing of tooth and bone implants, comprising the following steps:
a) providing a test system for the process;
b) providing a reference compound;
c) testing the reference compound in the test system and determining the reaction caused by the reference compound in the test system;
d) providing a candidate compound;
e) testing the candidate compound in the test system and determining the reaction caused by the candidate compound in the test system; and
f) comparing the reaction of the reference compound in the test system with the reaction of the candidate compound in the test system.

78. A method for the screening of a compound for promoting and/or inhibiting a process, whereby the process is selected from the group comprising tissue regeneration, repair of DNA damages, wound healing, cell mobility, angiogenesis in the wound area, epithelialization, tissue aging, inhibition of tissue aging, tissue rejuvenation, vascularization after cardial infarction and healing of tooth and bone implants, comprising the following steps:
a) providing a test system for the process;
b) providing a reference compound, whereby the reference compound comprises a label;
c) testing the reference compound in the test system and determining the reaction caused by the reference compound in the test system;
d) providing the candidate compound; and
e) testing the candidate compound in the test system, whereby the test system contains the reference compound, and determining the reaction of the test system, whereby the amount of released reference compound and/or the amount of the released label of the reference compound is determined.

79. A method for the screening of a compound for promoting and/or inhibiting a process, whereby the process is selected from the group comprising tissue regeneration, a repair of DNA damages, wound healing, cell mobility, angiogenesis in the wound area, epithelialization, tissue aging, inhibition of tissue aging, tissue rejuvenation, vascularization after cardial infarction and healing of tooth and bone implants, comprising the following steps:
a) providing a test system for the process;
b) providing a candidate compound, whereby the candidate compound comprises a label;
c) testing the candidate compound in the test system and determining the reaction caused by the candidate compound in the test system;
d) providing a reference compound; and
e) testing the reference compound in the test system, whereby the test system contains the candidate compound, and determining the reaction of the test system, whereby the amount of released candidate compound and/or the amount of released label of the candidate compound is determined.

80. The method according to any of claims 76 to 79, characterised in that the test system is an in vitro test system or an in vivo test system.

81. The method according to any of claims 76 to 80, characterised in that the reaction of the reference compound and/or of the candidate compound is a promotion of the process, and whereby preferably the candidate compound is a compound for promoting the process if the reaction of the candidate compound in the test system is equal to or more pronounced than the reaction of the reference compound.

82. The method according to any of claims 76 to 80, characterised in that the reaction of the reference compound and/or of the candidate compound is an inhibition of the process and whereby preferably the candidate compound is a compound for the inhibition of the process if the reaction of the test system caused by the candidate compound is a reaction which is inferior to the reaction of the test system caused by the reference compound.

83. The method according to any of claims 76 to 80, characterised in that the reference compound is a nucleic acid, the transcription product thereof and/or the translation product thereof, whereby the nucleic acid is selected from the group comprising genes for basic DNA-binding proteins, particularly as defined in any of the preceding claims.

84. Use of a method according to any of claims 76 to 83 for the screening of a compound for the treatment and/or prevention of a disease, whereby the test system provided is a test system for the respective disease.

85. Use according to claim 84, characterised in that the disease is selected from the group comprising those requiring repair of DNA damages, requiring tissue regeneration, requiring wound healing, requiring tooth and bone implants, those going along with tissue aging, wound healing disorders, skin diseases, xeroderma pigmentosum, leather skin, skin cancer, skin after sun burn, skin aging after sun burn, sun burn and cardial infarction.

86. Sun protection agent comprising at least a nucleic acid, the transcription product thereof and/or the translation product thereof, whereby the nucleic acid is selected from the group comprising genes for basic DNA-binding proteins.

87. Sun protection agent according to claim 86, characterised in that the basic DNA proteins are HMG proteins, particularly those described in any of the preceding claims.

88. Compound obtainable by a method according to any of claims 76 to 83 or a use according to claim 84 or 85.

89. Use of a compound according to claim 88 for the manufacture of a medicament, preferably for the treatment and/or prevention of a disease as described in any of the preceding claims.

90. A method for the treatment of an organism, characterised in that an effective amount of a DNA-binding protein, of a HMG protein, of a nucleic acid coding therefor or a transcription product thereof and/or a translation product thereof, a functional nucleic acid interacting therewith, a peptide interacting therewith or an antibody interacting therewith and/or a compound according to claim 89 is administered to the organism.

91. The method according to claim 90, characterised in that the organism is suffering from a disease or may suffer from said disease, or to fall ill with the disease, which is preferably a disease as described in any of the preceding claims.
